(19) United States
(12) Patent Application Publication
(10) Pub. No.: US 2012/0141523 A1
(43) Pub. Date: Jun. 7, 2012

(54) IMMUNOGENIC COMPOSITION COMPRISING ANTIGENIC S. AUREUS PROTEINS

(76) Inventors: Cindy Castado, Rixensart (BE); Sophie Marie Jeanne Valentine Germain, Rixensart (BE); Melanie Gilbert, Rixensart (BE)

(21) Appl. No.: 13/388,757

(22) PCT Filed: Aug. 3, 2010
(86) PCT No.: PCT/EP2010/061314

§ 371 Date: Feb. 13, 2012

(30) Foreign Application Priority Data
Aug. 5, 2009 (GB) 0913681.3

(51) Int. Cl. A61K 39/085; A61P 31/04; C07K 14/31

(52) U.S. Cl. 424/190.1; 530/350

(57) ABSTRACT

The present application relates to an immunogenic composition comprising a fragment of a staphylococcal Isd protein such as IsdA, IsdB, IsdC or IsdH which comprises a NEAT domain. Fusion proteins comprising a NEAT domain of a first staphylococcal Isd protein and a NEAT domain from a second Isd protein are also disclosed as well as fusion proteins comprising a NEAT domain of a staphylococcal Isd protein involved in an iron/heme uptake system and a ligand binding domain of a staphylococcal extracellular component binding protein, for example ClfA, ClfB, SdrC, SdrD or SdrE.

The present invention relates to the field of staphylococcal protein antigens, nucleic acids encoding them and immunogenic compositions and vaccines comprising such proteins or nucleic acids. The invention also relates to processes for manufacturing such compositions and their use in medicine.

Iron is an essential nutrient of almost all organisms and in bacterial infection, it is sequestered by host defence mechanisms to limite bacterial growth. To combat host iron restriction, bacterial pathogens have evolved multiple acquisition systems to obtain iron from host sources. Haem is the most prevalent form of iron in the human body, representing nearly 75% if the total iron.

The Isd (Iron-regulated surface determinant) system was identified as a primary haem acquisition pathway in staphylococci. Four wall-anchored proteins, IsdA (Mazmanian et al 2002, PNAS 99; 2293), IsdB (Mazmanian et al 2002, PNAS 99; 2293), IsdC (WO 06/59247) and IsdH or HarA (Dryla et al Molec. Microbiol. 2003, 49; 37-53) are proposed to act as receptors for haem.

Isd proteins have been proposed as vaccine candidates for use in a staphylococcal vaccine (WO 01/98499, WO 02/59148, WO 03/11899 and WO 06/59247).

S. aureus infections are treated with antibiotics, with penicillin being the drug of choice whereas vancomycin is used for methicillin resistant isolates. The percentage of staphylococcal strains exhibiting wide-spectrum resistance to antibiotics has become increasingly prevalent since the 1980's (Panlilo et al 1992, Infect. Control. Hosp. Epidemiol. 13; 582), posing a threat for effective antimicrobial therapy. In addition, the recent emergence of vancomycin resistant S. aureus strain has aroused fear that methicillin resistant S. aureus strains will emerge and spread for which no effective therapy is available.

An alternative approach of using antibodies against staphylococcal antigens in passive immunotherapy has been investigated. Therapy involving administration of polyclonal antisera are under development (WO 00/15238, WO 00/12132).

There remains a need to develop a vaccine which protects against staphylococcal disease. An approach using S. aureus capsular polysaccharide conjugates has failed to achieve regulatory approval (WO 03/61558) and a more complex vaccine containing additional staphylococcal components may be required to give effective protection.

Accordingly there is provided an immunogenic composition comprising a fragment of a staphylococcal Isd protein involved in an iron/heme uptake system which comprises a NEAT domain.

In a further aspect of the invention there is provided an immunogenic composition comprising a fragment of a staphylococcal Sdr family member (for example SdrC, SdrD, SdrE, SdrG, ClfA or ClfB) which comprises a ligand binding domain (for example an N23 domain).

In a further aspect of the invention there is provided a fusion protein comprising a NEAT domain of a staphylococcal Isd protein involved in an iron/heme uptake system and a ligand binding domain of a staphylococcal extracellular component binding protein (for example an N23 domain).

In a further aspect of the invention there is provided a fusion protein comprising a NEAT domain of a first staphylococcal Isd protein and a NEAT domain from a second Isd protein.

In a further aspect of the invention there is provided a polynucleotide comprising a polynucleotide sequence encoding a NEAT domain of a staphylococcal Isd protein and a polynucleotide sequence encoding a ligand binding domain of a staphylococcal extracellular component binding protein.

In a further aspect of the invention there is provided a vaccine comprising the immunogenic composition, the fusion protein or the polynucleotide of the invention and a pharmaceutically acceptable excipient.

In a further aspect of the invention there is provided a process for making the vaccine of the invention comprising the step of adding a pharmaceutically acceptable excipient to the immunogenic composition, the fusion protein or the polynucleotide of the invention.

In a further aspect of the invention there is provided a immunogenic composition of the invention for use in the treatment or prevention of staphylococcal infection or disease.

In an further aspect of the invention there is provided a use of the immunogenic composition or the fusion protein or the polynucleotide of the invention in the preparation of a medicament for the treatment or prevention of staphylococcal disease.

In a further aspect of the invention there is provided a method of treating or preventing staphylococcal disease comprising administering the immunogenic composition, the fusion protein or the polynucleotide of the invention to a patient in need thereof.

### DESCRIPTION OF THE FIGURES

FIG. 1 Schematic showing structure of native IsdA and NEAT domain fragments of IsdA.

FIG. 2 Schematic showing native IsdB and fragments of IsdB. GGS indicates the amino acid sequence glycine, glycine, serine.

FIG. 3 Schematic of IsdA/IsdB fusion proteins. GGS indicates the amino acid sequence glycine, glycine, serine and IsdH linker indicates the sequence of SEQ ID NO:98.

FIG. 4 Bar chart showing the results of ELISA assays measuring the level of the anti-IsdA immune response following immunisation of mice with native proteins, fragments and fusion proteins.

FIG. 5 Bar chart showing the results of ELISA assays measuring the level of the anti-IsdB immune response following immunisation of mice with native proteins, fragments and fusion proteins.

FIG. 6 Bar chart showing the results of an opsonophagosytosis assay, measuring the immune response generated in pooled mice sera following three innocuations with native proteins, fragments and fusions.

FIG. 7 Graph showing the results of an adhesion assay in which fibrinogen adhesion to ClfA coated plates is measured. The diamond marked line shows the binding of fibrinogen to ClfA N123 474 mutant and the square marked line shows the binding of fibrinogen to wildtype ClfA N123.

FIG. 8 Graph showing the results of an adhesion assay in which ClfA adhesion to fibrinogen coated plates is measured. The darker diamond marked line shows the binding of Wildtype N123 ClfA to fibrinogen, the lighter diamond marker line shows the binding of 474 mutant ClfA N123 to fibrinogen and the square marked line shows the negative control

FIG. 9 Graph showing the ability of antibodies raised against wild type of 474 mutant ClfA N123 to inhibit the binding of fibrinogen to N123 ClfA coated plates.

FIG. 10 Graph showing the ability of antibodies raised against wild type and 474 mutant ClfA to inhibit the binding of S. aureus bacteria to N123 ClfA coated plates.

### DETAILED DESCRIPTION

The present invention discloses an immunogenic composition comprising a fragment of a staphylococcal Isd protein involved in an iron/heme uptake system which comprises a NEAT domain.

All fragments described herein are immunogenic fragments which may be capable of generating a specific immune response against at least one Gram positive bacterium, for example S. aureus or S. epidermidis. They contain for example at least 10, 20, 30, 40, 50, 60, 70, 80, 90 or 100 amino acids.

A Staphylococcal Isd protein may be from any staphylococcal bacterium, for example from Staphylococcus aureus or a coagulase negative staphylococcus e.g. Staphylococcus epidermidis.

An isd (iron-regulated surface determinant) protein belongs to the family of proteins involved in iron acquisition and include IsdA, IsdB, IsdC, IsdH (or HarA), IsdD, IsdE, IsdF, IsdG and IsdI.

The immunogenic compositions of the invention comprise a fragment of a staphylococcal Isd protein which comprises a NEAT domain (Grigg et al Molecular Biology 2007, 63; 139-149). These domains are approximately 125 amino acids long and are named because of the chromosomal location NEAr iron Transport protein encoding genes. Between one and five NEAT domains are found in some proteins terminating in cell wall anchoring motifs. IsdA and IsdC contain one NEAT domain, IsdB contains 2 NEAT domains, (either or both may be included in the immunogenic composition of the invention) and IsdH contains 3 NEAT domains (any of which may be included in the immunogenic composition of the invention). In an embodiment, the NEAT domain is a complete NEAT domain or a complete NEAT domain comprising at least 100, 110, 115, 120, 122, 123, 124, 125, 130, 135 or 140 amino acids. The fragment is an immunogenic fragment which is for example, capable of generating an anti-staphylococcal immune response as measured in an assay such as an ELISA or serum bacteriocidal assay, or in an assay demonstrating protection against challenge with a staphylococcal infection, for example S. aureus.

The immunogenic composition of the invention optionally comprises the NEAT domain of IsdA which starts at amino acid 40, 45, 50, 55, 56, 57, 58, 59, 60, 61, 62, 63 or 64 and extends to amino acid 182, 183, 184, 185, 186, 187, 188, 189 or 190, 195 or 200. In an example the IsdA NEAT domain is amino acids 58-188, 59-187, 60-186, 61-185 or 62-184 of IsdA, optionally having the polypeptide sequence sharing 80%, 85%, 90%, 95%, 97%, 98%, 99% or 100% identity with SEQ ID NO:1, 2, 3, 4 or 5. Throughout the specification, the amino acid numbers relating to the start and end of an IsdA fragments are in respect to SEQ ID NO:1.

The immunogenic composition of the invention optionally comprises one or two NEAT domains of IsdB. The first NEAT domain of IsdB starts at amino acid 138, 139, 140, 141, 142, 143, 144, 145 or 146 and extends to amino acid 263, 264, 265, 266, 267, 268, 269, 270, 271, or 272 of IsdB, optionally having the polypeptide sequence of SEQ ID NO:6, 7 or 8. The second NEAT domain of IsdB starts at amino acid 334, 335, 336, 337, 338, 339, 340, 341, 342, 343 or 344 and extends to amino acid 458, 459, 460, 461, 462, 463, 464, 465 or 466 of IsdB, optionally having the polypeptide sequence of SEQ ID NO:6, 9 or 10. In an embodiment the IsdB NEAT domain is amino acids 138-271, 140-169, 142-267, 335-464, 337-462 or 339-460 of IsdB. In an example the IsdB NEAT domain fragment is from amino acid 138-464, 140-462 or 142-464.

In embodiments containing two IsdB NEAT domains, the amino acids between the two NEAT domains are either retained or deleted and/or replaced with a linker x which consists of a peptide bond or any other covalent linkage or a peptide chain containing more than 2, 4, 6, 8, 10, 20, 50, 100 or 200 amino acids. In an embodiment, the linker x comprises a tripeptide such as glycine-glycine-serine (GGS). In a further example, the linker x comprises SEQ ID NO:98. In an embodiment, amino acids 267-339, 269-341 or 271-343 are deleted and optionally replaced with a linker x. In an example the immunogenic composition of the invention comprises two IsdB NEAT domains and the fragment is from amino acid 42-486, 42-462, 42-461, 82-486, 82-462 or 82-461.

The IsdB NEAT domain(s) optionally contains a sequence sharing 80%, 85%, 90%, 95%, 97%, 98%, 99% or 100% identity with SEQ ID NO: 6, 7, 8, 9, 10, 11, 12, 13 or 86 or of sequences disclosed in WO 05/09379. Through out the specification, amino acid numbers relating to the start and end of an IsdB fragments are in respect to SEQ ID NO:6.

The immunogenic composition of the invention optionally comprises the NEAT domain of IsdC which starts at amino acid 20, 21, 22, 23, 24, 25, 26, 27, 28, 29 or 30 and extends to amino acid 149, 150, 151, 152, 153, 154, 155, 156, 157 or 158. In an example the IsdC NEAT domain is amino acids 21-156, 22-155, 23-154, 28-154, 24-153 or 25-152 of IsdC, optionally having the polypeptide sequence of SEQ ID NO:14. The IsdC NEAT domain optionally contains a sequence sharing sharing 80%, 85%, 90%, 95%, 97%, 98%, 99% or 100% identity with SEQ ID NO: 14 or 15. Through out the specification, amino acid numbers relating to the start and end of an IsdC fragments are in respect to SEQ ID NO:14.

The immunogenic composition of the invention optionally comprises one, two or three NEAT domains of IsdH. The first NEAT domain of IsdH starts at amino acid 97, 98, 99, 100, 101, 102, 103, 104 or 105 and extends to amino acid 228, 229, 230, 231, 232, 233, 234, 235. 236, 237 or 238 of IsdH. The second NEAT domain of IsdH starts at amino acid 337, 338, 339, 340, 341, 342, 343, 344 or 345 and extends to amino acid 466, 467, 468, 469, 470, 471, 472, 473, 474 or 475 of IsdH. The third NEAT domain of IsdH starts at amino acid 535, 536, 537, 538, 539, 540, 541, 542, 543, 544 or 545 and extends to amino acid 660, 661, 662, 663, 664, 665, 666, 667, 668, 669 of 670 of IsdH. In each case IsdH optionally has the polypeptide sequence of SEQ ID NO:16. In an example the IsdH NEAT domain is amino acids 99-234, 100-233, 101-232, 102-231, 103-230, 339-473, 342-470-341-471, 342-470, 343-469, 537-666, 538-665, 539-664, 540-663 or 538-662 of IsdH. In an embodiment, the IsdH NEAT domain fragment is from amino acid 99-473, 101-471, 103-469, 99-666, 101-664, 103-662, 339-666, 341-664 or 343-662, optionally having the amino acids between the two or three NEAT domains deleted and/or replaced with a linker x which consists of a peptide bond or any other covalent linkage or a peptide chain containing more than 2, 4, 6, 8, 10, 20, 50, 100 or 200 amino acids. In an embodiment, the linker x comprises a tripeptide such as glycine-glycine-serine (GGS). In a further example, the linker x comprises SEQ ID NO:98. For example, amino acids 230-343, 232-341, 234-339, 469-541, 471-539 or 473-537 are deleted. The IsdH NEAT domain(s) optionally contains a sequence sharing sharing 80%, 85%, 90%, 95%, 97%, 98%, 99% or 100% identity with SEQ ID NO: 16, 17, 18 or 19. Through out the specification, amino acid numbers relating to the start and end of an IsdH fragments are in respect to SEQ ID NO:16.

In an embodiment, the immunogenic composition of the invention comprises 2, 3, 4, 5 or 6 fragments of Isd protein(s) which comprise a NEAT domain. For example fragments comprising IsdA and 1 IsdB NEAT domains; IsdA and 2 IsdB NEAT domains; IsdA and IsdC NEAT domains, IsdA and 1 IsdH NEAT domains; IsdA and 2 IsdH NEAT domains; IsdA and 3 IsdH NEAT domains; 1 IsdB and IsdC NEAT domains, 2 IsdB and IsdC NEAT domains; 1 IsdB and 1 IsdH NEAT Domains; 2 IsdB and 1 IsdH NEAT domains; 1 IsdB and 2 IsdH NEAT Domains; 2 IsdB and 2 IsdH NEAT domains; 1 IsdB and 3 IsdH NEAT Domains; 2 IsdB and 3 IsdH NEAT domains; IsdC and 1 IsdH NEAT domains; IsdC and 2 IsdH NEAT domains; IsdC and 3 IsdH NEAT domains; IsdA, 1 IsdB and IsdC NEAT domains; IsdA, 2 IsdB and IsdC NEAT domains; IsdA, IsdC and 1 IsdH NEAT domains; IsdA, IsdC and 2 IsdH NEAT domains; IsdA, IsdC and 3 IsdH NEAT domains; IsdB, IsdC and IsdH domains; IsdA, IsdB and IsdH NEAT domains or IsdA, IsdB, IsdC and IsdH NEAT domains.

In an embodiment, the immunogenic composition of the invention comprises a staphylococcal extracellular component binding protein or fragment thereof selected from the group consisting of laminin receptor, SitC/MntC/saliva binding protein, EbhA, EbhB, Elastin binding protein (EbpS), EFB (FIB), SBI, autolysin, ClfA, SdrC, SdrD, SdrE, SdrG, SdrH, Protein A, Lipase GehD, SasA, FnbA, FnbB, Cna, ClfB, FbpA, Npase, IsaA/PisA, SsaA, EPB, SSP-1, SSP-2, Vitronectin binding protein, fibrinogen binding protein, coagulase, Fig and MAP (WO 06/32475, WO 06/32472, WO 06/32500).

In an embodiment, the staphylococcal extracellular component binding protein is selected from the group consisting of ClfA, ClfB, SdrD, SdrE, SdrG or SdrC or immunogenic fragment thereof. Where the protein is ClfA, either wildtype or ClfA containing a mutation at residue Y474, D321, P336 and/or Y338 can be used. Y474 may be substuted with a different amino acid, for example histidine. D321 may be substituted with a different amino acid, for example tyrosine. P336 may be substituted with a different amino acid, for example serine. Y338 may be substituted with a different amino acid, for example alanine.

All fragments described herein are optionally immunogenic fragments. An immunogenic fragment is capable of generating an immune response, optionally a protective immune response against a staphylococcal, optionally a S. aureus strain. The immunogenic fragment contains at least 10, 20, 30, 40, 50, 70 or 100 contiguous amino acids.

In an embodiment, the staphylococcal extracellular binding component is an immunogenic fragment comprising a ligand binding region.

A ligand binding region is a domain which binds to an extracellular component for example fibrinogen. The ligand binding region (A region) of ClfA, ClfB, SdrC, SdrD, SdrE and SdrG is in the N-terminal half of the polypeptide (McCrea et al Microbiology 2000, 146; 1535-1546). It is between 200-600 amino acids in length and contains N1, N2 and N3 domains (Clarke and Foster 2006 Advances in Microbial Physiology 51; 187-224; Perkins et al 2001 J. Biol. Chem. 276; 44721-44728).

The immunogenic composition of the invention optionally comprises a N1 domain of ClfA which starts at amino acid 38, 39, 40, 41, 42, 43, 44 or 45 and extends to amino acid 214, 215, 216, 217, 218, 219, 220, 221 or 222. In an example the ClfA N1 domain is amino acids 38-219, 39-218, 40-217, 41-216 or 42-215 of ClfA, optionally having the polypeptide sequence of SEQ ID NO:20. The ClfA NEAT domain optionally contains a sequence sharing sharing 80%, 85%, 90%, 95%, 97%, 98%, 99% or 100% identity with SEQ ID NO:21, 22, 23 or 24. Through out the specification, amino acid numbers relating to the start and end of an ClfA fragments are in respect to SEQ ID NO:20.

The immunogenic composition of the invention optionally comprises a N1 domain of ClfB which starts at amino acid 48, 49, 50, 51, 52, 53, 54 or 55 and extends to amino acid 266, 267, 268, 269, 270, 271, 272, 273 or 274. In an example the ClfB N1 domain is amino acids 53-272, 52-272, 51-272, 50-272 or 51-272 of ClfB, optionally having the polypeptide sequence of SEQ ID NO:25. The ClfB NEAT domain optionally contains a sequence sharing sharing 80%, 85%, 90%, 95%, 97%, 98%, 99% or 100% identity with SEQ ID NO: 26, 27 or 28. Through out the specification, amino acid numbers relating to the start and end of an ClfB fragments are in respect to SEQ ID NO:25.

The immunogenic composition of the invention optionally comprises a N1 domain of SdrG which starts at amino acid 48, 49, 50, 51, 52, 53, 54 or 55 and extends to amino acid 260, 261, 262, 263, 264, 265, 266, 267, 268, 269, 270, 271, 272, 273 or 274. In an example the SdrG N1 domain is amino acids 53-261, 52-267, 52-261 or 53-267 of SdrG, optionally having the polypeptide sequence of SEQ ID NO:41 or 44. The SdrG N1 domain optionally contains a sequence sharing sharing 80%, 85%, 90%, 95%, 97%, 98%, 99% or 100% identity with SEQ ID NO:41, 42, 44 or 45. Through out the specification, amino acid numbers relating to the start and end of an SdrG fragments are in respect to SEQ ID NO:41 in relation to S. aureus and SEQ ID NO:44 in relation to S. epidermidis.

The immunogenic composition of the invention optionally comprises a N1 domain of SdrD which starts at amino acid 50, 51, 52, 53, 54, 55, 56 or 57 and extends to amino acid 228, 229, 230, 231, 232, 233, 234, 235 or 236. In an example the SdrD N1 domain is amino acids 55-233, 54-233, 53-233, 52-233 or 51-233 of SdrD, optionally having the polypeptide sequence of SEQ ID NO:35. The SdrD N1 domain optionally contains a sequence sharing sharing 80%, 85%, 90%, 95%, 97%, 98%, 99% or 100% identity with SEQ ID NO:35 or 36. Through out the specification, amino acid numbers relating to the start and end of an SdrD fragments are in respect to SEQ ID NO:35.

The immunogenic composition of the invention optionally comprises a N1 domain of SdrE which starts at amino acid 50, 51, 52, 53, 54, 55, 56 or 57 and extends to amino acid 257, 258, 259, 260, 261, 262, 263, 264 or 265. In an example the SdrE N1 domain is amino acids 55-261, 54-261, 53-261, 52-261 or 51-261 of SdrE, optionally having the polypeptide sequence of SEQ ID NO:38 or 39. The SdrE N1 domain optionally contains a sequence sharing sharing 80%, 85%, 90%, 95%, 97%, 98%, 99% or 100% identity with SEQ ID NO:38 or 39. Through out the specification, amino acid numbers relating to the start and end of an SdrE fragments are in respect to SEQ ID NO:38.

The immunogenic composition of the invention optionally comprises a N1 domain of SdrC which starts at amino acid 47, 48, 49, 50, 51, 52, 53, 54, 55, 56 or 57 and extends to amino acid 171, 172, 173, 174, 175, 176, 177, 178, 179 or 180. In an example the SdrC N1 domain is amino acids 51-174, 51-175, 51-176, 51-177 or 51-178 of SdrC, optionally having the polypeptide sequence of SEQ ID NO:29 or 32. The SdrC N1 domain optionally contains a sequence sharing sharing 80%, 85%, 90%, 95%, 97%, 98%, 99% or 100% identity with SEQ ID NO:29, 30, 32 or 33. Through out the specification, amino acid numbers relating to the start and end of an SdrC fragments are in respect to SEQ ID NO29.

The immunogenic composition of the invention optionally comprises a N23 domain of ClfA which starts at amino acid 214, 215, 216, 217, 218, 219, 220, 221 or 222 and extends to amino acid 554, 555, 556, 557, 558, 559 or 560. In an example the ClfA N23 domain is amino acids 214-559, 215-559, 216-559, 216-558 or 216-557 of ClfA, optionally having the polypeptide sequence of SEQ ID NO:20. The ClfA N23 domain optionally contains a sequence sharing sharing 80%, 85%, 90%, 95%, 97%, 98%, 99% or 100% identity with SEQ ID NO:20, 21, 22, 23 or 24.

The immunogenic composition of the invention optionally comprises a N23 domain of ClfB which starts at amino acid 193, 194, 195, 196, 197, 198, 199 or 200 and extends to amino acid 538, 539, 540, 541, 542, 543, 544 or 545. In an example the ClfB N23 domain is amino acids 197-538, 197-539, 197-540, 197-541 or 197-542 of ClfB, optionally having the polypeptide sequence of SEQ ID NO:25. The ClfB N23 domain optionally contains a sequence sharing sharing 80%, 85%, 90%, 95%, 97%, 98%, 99% or 100% identity with SEQ ID NO:25, 26, 27 or 28.

The immunogenic composition of the invention optionally comprises a N23 domain of SdrG which starts at amino acid 261, 262, 263, 264, 265, 266, 267, 268, 269, 270, 271, 272, 273, 274, 275 or 276 and extends to amino acid 524, 525, 526, 527, 528, 529, 530. In an example the SdrG N23 domain is amino acids 267-526, 268-526, 262-528, 268-527 or 268-525 of SdrG, optionally having the polypeptide sequence of SEQ ID NO:41 or 44. The SdrG N23 domain optionally contains a sequence sharing sharing 80%, 85%, 90%, 95%, 97%, 98%, 99% or 100% identity with SEQ ID NO:41, 42, 43, 44, 45 or 46.

The immunogenic composition of the invention optionally comprises a N23 domain of SdrD which starts at amino acid 230, 231, 232, 233, 234, 235, 236, 237 or 238 and extends to amino acid 564, 565, 566, 567, 568, 569, 570, 571 or 572. In an example the SdrD N23 domain is amino acids 234-566, 234-567, 234-568, 234-569 or 234-570 of SdrD, optionally having the polypeptide sequence of SEQ ID NO:35. The SdrD N23 domain optionally contains a sequence sharing sharing 80%, 85%, 90%, 95%, 97%, 98%, 99% or 100% identity with SEQ ID NO:35, 36 or 37.

The immunogenic composition of the invention optionally comprises a N23 domain of SdrE which starts at amino acid 259, 260, 261, 262, 263, 264, 265 or 266 and extends to amino acid 592, 593, 594, 595, 596, 597, 598 or 599. In an example the SdrE N23 domain is amino acids 262-592, 262-593, 262-594, 262-595 or 262-596 of SdrE, optionally having the polypeptide sequence of SEQ ID NO:38. The SdrE N23 domain optionally contains a sequence sharing sharing 80%, 85%, 90%, 95%, 97%, 98%, 99% or 100% identity with SEQ ID NO:38, 39 or 40.

The immunogenic composition of the invention optionally comprises a N23 domain of SdrC which starts at amino acid 172, 173, 174, 175, 176, 177, 178, 179 or 180 and extends to amino acid 424, 425, 426, 427, 428, 429, 430, 431 or 432. In an example the SdrC N23 domain is amino acids 176-426, 176-427, 176-428, 176-429 or 176-430 of SdrC, optionally having the polypeptide sequence of SEQ ID NO:29 or 32. The SdrC N23 domain optionally contains a sequence sharing sharing 80%, 85%, 90%, 95%, 97%, 98%, 99% or 100% identity with SEQ ID NO:29, 30, 31, 32, 33 or 34.

In an embodiment, the ligand binding region comprises or consists of the N2, N3, N1, N2N3 or N1N2N3 domain(s).

An immunogenic composition comprising a fragment of ClfB, SdrC, SdrD, SdrE or SdrG which comprises a N23 domain is a further independent aspect of the invention. The N23 domain of this independent embodiment may comprise any of the features described above.

In an embodiment, the immunogenic composition of the invention comprises the N23 domain of 1, 2, 3, 4, 5 or 6 of ClfA, ClfB, SdrC, SdrD, SdrE or SdrG. For example, the immunogenic composition may contain the N23 domains of ClfA and ClfB, ClfA and SdrC, ClfA and SdrD, ClfA and SdrE, ClfA and SdrG, ClfB and SdrC, ClfB and SdrD, ClfB and SdrE, ClfB and SdrG, SdrC and SdrD, SdrC and SdrE, SdrC and SdrG, SdrD and SdrE, SdrD and SdrG or SdrE and SdrG.

In an embodiment, the immunogenic composition of the invention comprises a fragment of a staphylococcal extracellular binding component, for example an adhesin, comprising a ligand binding region has an amino acid sequence having at least 85%, 90%, 95%, 96%, 97%, 98% 99% or 100% identity to an amino acids sequence selected from the group consisting of SEQ ID NO: 21, 22, 23, 24, 26, 27, 28, 30, 31, 33, 34, 36, 37, 39, 40, 42, 43, 45 or 46. The fragment is an immunogenic fragment which is for example, capable of generating an anti-staphylococcal immune response as measured in an assay such as an ELISA or serum bacteriocidal assay, or in an assay demonstrating protection against challenge with a staphylococcal infection, for example S. aureus.

In an embodiment, the immunogenic composition of the invention comprises the fragment of a staphylococcal Isd protein which is covalently linked to a staphylococcal extracellular component binding protein or fragment thereof.

A further aspect of the invention is a fusion protein comprising a NEAT domain of a staphylococcal Isd protein involved in an iron/heme uptake system and a ligand binding domain of a staphylococcal extracellular component binding protein.

A fusion protein contains amino acid sequences from at least two different proteins covalently linked into the same polypeptide chain. The amino acid sequences from at least two different proteins may be directly linked by a peptide bond or any other covalent linkage or may be linked through a linker x which is optionally a peptide chain containing more than 2, 4, 6, 8, 10, 20, 50, 100 or 200 amino acids. In an embodiment, the linker x comprises a tripeptide such as glycine-glycine-serine (GGS). In a further example, the linker x comprises SEQ ID NO:98. Where two or more linker x sequences fuse three or more protein sequences together, the same linker x or different linker x sequences may be used. In an embodiment where the linker x is less than 10 amino acids long, the same linker is used. Where the linker x is more than 10, 20, 50 or 100 amino acids long, different linker x sequences are present in the fusion protein.

In an embodiment the fusion protein of the invention contains a NEAT domain from a S. aureus Isd protein optionally selected from the group consisting of IsdA, IsdB, IsdC and IsdH.

In an embodiment the fusion protein of the invention contains a ligand binding domain (for example the N1, N2, N3, N23 or N123 domain) from a S. aureus protein optionally selected from the group consisting of ClfA, ClfB, SdrC, SdrD and SdrE or from a S. epidermidis protein such as SdrG.

In an independent embodiment, the invention includes a fusion protein comprising a NEAT domain of a first staphylococcal Isd protein and a NEAT domain from a second Isd protein. For example from IsdA and IsdB, IsdA and IsdC, IsdA and IsdH, IsdB and IsdC, IsdB and IsdH, IsdC and IsdH.

In an embodiment, the fusion protein of the invention has an amino acid sequence containing an amino acid sequence having at least 85%, 90%, 95%, 96%, 97%, 98%, 99% or 100% identity to an amino acid sequence selected from the group consisting of SEQ ID NO: 47-97. In these sequences, x denotes either a covalent bond or 1-3, 1-5, 1-10, 1-20, 1-50, 1-100, 90-120, 1-200 or 1-500 amino acids. In an embodiment, x denotes 3 amino acids, optionally having the sequence GGS. In an embodiment x denotes 108 amino acids having the sequence:

SEQ ID NO: 98

DTNDAVVTNDQSSSVASNQTNTNTSNQNISTINNANNQPQATTNMSQP
AQPKSSTNADQASSQPAHETNSNGNTNDKTNESSNQSDVNQQYPPA
DESLQDAIKNPAII

Where two linkers, x, are denoted, the linkers may be the same or different. For example, the first and second domains may be separated by a GGS linker and the second and third domains are separated by a 108 amino acid SEQ ID NO:98 sequence. Alternatively, the first and second domains may be separated by a GGS linker and the second and third domains are separated by a GGS linker. Alternatively, the first and second domains may be separated by a 108 amino acid SEQ ID NO:98 sequence and the second and third domains are separated by a GGS linker.

In an independent embodiment, the invention includes a fusion protein comprising a ligand binding domain of a first staphylococcal extracellular component binding protein and a ligand binding domain of a second staphylococcal extracellular component binding protein. For example, the fusion protein may contain ligand binding domains from SdrC and SdrD, SdrC and SdrE, SdrC and SdrG, SdrC and ClfA, SdrC and ClfB, SdrD and SdrE, SdrD and SdrG, SdrD and ClfA, SdrD and ClfB, SdrE and SdrG, SdrE and ClfA, SdrE and ClfB, SdrG and ClfA, SdrG and ClfB or ClfA and ClfB. The ligand binding domain optionally consists of a N1, N2, N3, N2N3 or N1N2N3 domain and the fusion protein optionally contains the same domain type for each protein, for example N2N3 domains for SdrC and SdrD, SdrC and SdrE, SdrC and SdrG, SdrC and ClfA, SdrC and ClfB, SdrD and SdrE, SdrD and SdrG, SdrD and ClfA, SdrD and ClfB, SdrE and SdrG, SdrE and ClfA, SdrE and ClfB, SdrG and ClfA, SdrG and ClfB or ClfA and ClfB.

In an embodiment, the fusion protein comprises a ligand binding domain from 3, 4, 5 or 6 staphylococcal extracellular component binding proteins. For example, comprising ClfA, ClfB and SdrC; ClfA, ClfB and SdrD, ClfA, ClfB and SdrE; ClfA, ClfB and SdrG; ClfA, SdrC and SdrG; ClfA, SdrC and SdrD; ClfA, SdrC and SdrE, ClfA, SdrD and SdrE; ClfA, SdrD and SdrG; ClfA, SdrE and SdrG; ClfB, SdrC and SdrD; ClfB, SdrC and SdrG; ClfB, SdrC and SdrE; ClfB, SdrD and SdrE; ClfB, SdrD and SdrG; ClfB, SdrE and SdrG; SdrC, SdrD and SdrE; SdrC, SdrD and SdrG; SdrD, SdrE and SdrG.

All fragments or fusion proteins of the invention may be isolated or purified, for example from host cell used for expression.

In each case, the sequences of the NEAT domains or ligand binding domains are optionally as set out above.

In an embodiment, the immunogenic composition of the invention comprises a fusion protein of the invention.

In an embodiment, the immunogenic composition of the invention comprises a further staphylococcal antigen, for example a saccharide as described below. In embodiments of the invention where a saccharide antigen is present, the fragments or fusion proteins of the invention are optionally present as free or un-conjugated proteins. Optionally they are conjugated to a saccharide antigen as a carrier protein.

Saccharides

Poly N-acetylated Glucosamine (PNAG)

PNAG is a polysaccharide intercellular adhesin and is composed of a polymer of β-(1→6)-linked glucosamine, optionally substituted with N-acetyl and/or O-succinyl constituents. This polysaccharide is present in both S. aureus and S. epidermidis and can be isolated from either source (Joyce et al 2003, Carbohydrate Research 338; 903; Maira-Litran et al 2002, Infect. Imun. 70; 4433). For example, PNAG may be isolated from S. aureus strain MN8m (WO 04/43407). The preparation of dPNAG is described in WO 04/43405.

The polysaccharide previously known as poly-N-succinyl-β-(1→6)-glucosamine (PNSG) was recently shown not to have the expected structure since the identification of N-succinylation was incorrect (Maira-Litran et al 2002, Infect. Imun. 70; 4433). Therefore the polysaccharide formally known as PNSG and now found to be PNAG is also encompassed by the term PNAG.

PNAG may be of different sizes varying from over 400 kDa to between 75 and 400 kDa to between 10 and 75 kDa to oligosaccharides composed of up to 30 repeat units (of β-(1→6)-linked glucosamine, optionally substituted with N-acetyl and O-succinyl constituents). Any size of PNAG polysaccharide or oligosaccharide may be use in an immunogenic composition of the invention, for example a size of over 40 kDa can be used. Sizing may be achieved by any method known in the art, for instance by microfluidisation, ultrasonic irradiation or by chemical cleavage (WO 03/53462, EP497524, EP497525).

Size ranges of PNAG are for example 40-400 kDa, 50-350 kDa, 40-300 kDa, 60-300 kDa, 50-250 kDa and 60-200 kDa.

PNAG can have different degree of acetylation due to substitution on the amino groups by acetate. PNAG produced in vitro is almost fully substituted on amino groups (95-100%). Alternatively, a deacetylated PNAG can be used having less than 50%, 40%, 30%, 20%, 10% or 5% N-acetylation. Use of a deacetylated PNAG allows opsonic killing of Gram positive bacteria, optionally S. aureus and/or S. epidermidis (WO 04/43405). In an embodiment, the PNAG has a size between 40 kDa and 300 kDa and is deacetylated so that less than 50%, 40%, 30%, 20%, 10% or 5% of amino groups are N acetylated.

In an embodiment, the PNAG is not O-succinylated or is O-succinylated on less than 25, 20, 15, 10, 5, 2, 1 or 0.1% of residues.

The term deacetylated PNAG (dPNAG) refers to a PNAG polysaccharide or oligosaccharide in which less than 50%, 40%, 30%, 20%, 10% or 5% of the amino groups are acetylated.

As used herein, the term PNAG encompasses both acetylated and deacetylated forms of the saccharide.

In an embodiment, PNAG is deacetylated to form dPNAG, by chemically treating the native polysaccharide. For example, the native PNAG is treated with a basic solution such that the pH rises to above 10. For instance the PNAG is treated with 0.1-5M, 0.2-4M, 0.3-3M, 0.5-2M, 0.75-1.5M or 1M NaOH, KOH or NH₄OH. Treatment is for at least 10 or 30 minutes, or 1, 2, 3, 4, 5, 10, 15 or 20 hours at a temperature of 20-100, 25-80, 30-60 or 30-50 or 35-45° C. dPNAG may be prepared as described in WO 04/43405.

In an embodiment, the polysaccharide(s) included in the immunogenic composition of the invention are conjugated to a carrier protein as described below or alternatively unconjugated.

Type 5 and Type 8 Polysaccharides from S. aureus

Most strains of S. aureus that cause infection in man contain either Type 5 or Type 8 polysaccharides. Approximately 60% of human strains are Type 8 and approximately 30% are Type 5. The structures of Type 5 and Type 8 capsular polysaccharide antigens are described in Moreau et al Carbohydrate Res. 201; 285 (1990) and Fournier et al Infect. Immun. 45; 87 (1984). Both have FucNAcp in their repeat unit as well as ManNAcA which can be used to introduce a sulfhydryl group.

Recently (Jones Carbohydrate Research 340, 1097-1106 (2005)) NMR spectroscopy revised the structures of the capsular polysaccharides to:

Type 5

→4)-β-D-ManNAcA-(1→4)-α-L-FucNAc(3OAc)-(1→3)-β-D-FucNAc-(1→

Type 8

→3)-β-D-ManNAcA(4OAc)-(1→3)-α-L-FucNAc(1→3)-α-D-FucNAc(1→

Polysaccharides may be extracted from the appropriate strain of S. aureus using methods well known to the skilled man, for instance as described in U.S. Pat. No. 6,294,177 or Infection and Immunity (1990) 58(7); 2367. For example, ATCC 12902 is a Type 5 S. aureus strain and ATCC 12605 is a Type 8 S. aureus strain.

Polysaccharides are of native size or alternatively may be sized, for instance by microfluidisation, ultrasonic irradiation or by chemical treatment. The invention also covers oligosaccharides derived from the type 5 and 8 polysaccharides from S. aureus.

The weight-average molecular weight of the saccharide may be 1000-2000000, 5000-1000000, 10000-500000, 50000-400000, 75000-300000, or 100000-200000. The molecular weight or average molecular weight of a saccharide herein refers to the weight-average molecular weight (Mw) of the saccharide measured prior to conjugation and is measured by MALLS. The MALLS technique is well known in the art and is typically carried out as described in example 2. For MALLS analysis of saccharides, two columns (TSKG6000 and 5000PWxI) may be used in combination and the saccharides are eluted in water. Saccharides are detected using a light scattering detector (for instance Wyatt Dawn DSP equipped with a 10 mW argon laser at 488 nm) and an inferometric refractometer (for instance Wyatt Otilab DSP equipped with a P100 cell and a red filter at 498 nm). In an embodiment, the polydispersity of the saccharide is 1-1.5, 1-1.3, 1-1.2, 1-1.1 or 1-1.05 and after conjugation to a carrier protein, the polydispersity of the conjugate is 1.0-2.5, 1.0-2.0. 1.0-1.5, 1.0-1.2, 1.5-2.5, 1.7-2.2 or 1.5-2.0. All polydispersity measurements are by MALLS.

The type 5 and/or 8 capsular polysaccharide or oligosaccharides included in the immunogenic composition of the invention are O-acetylated. In an embodiment, the degree of O-acetylation of type 5 capsular polysaccharide or oligosaccharide is 10-100%, 20-100%, 30-100%, 40-100%, 50-100%. 60-100%, 70-100%, 80-100%, 90-100%, 50-90%, 60-90%, 70-90% or 80-90%. In an embodiment, the degree of O-acetylation of type 8 capsular polysaccharide or oligosaccharide is 10-100%, 20-100%, 30-100%, 40-100%, 50-100%. 60-100%, 70-100%, 80-100%, 90-100%, 50-90%, 60-90%, 70-90% or 80-90%. In an embodiment, the degree of O-acetylation of type 5 and type 8 capsular polysaccharides or oligosaccharides is 10-100%, 20-100%, 30-100%, 40-100%, 50-100%. 60-100%, 70-100%, 80-100%, 90-100%, 50-90%, 60-90%, 70-90% or 80-90%.

The degree of O-acetylation of the polysaccharide or oligosaccharide can be determined by any method known in the art, for example, by proton NMR (Lemercinier and Jones 1996, Carbohydrate Resarch 296; 83-96, Jones and Lemercinier 2002, J Pharmaceutical and Biomedical analysis 30; 1233-1247, WO 05/033148 or WO 00/56357). A further commently used method is that described by Hestrin (1949) J. Biol. Chem. 180; 249-261.

O-acetyl groups can be removed by hydrolysis, for example by treatment with a base such as anhydrous hydrazine (Konadu et al 1994; Infect. Immun. 62; 5048-5054) or treatment with 0.1N NaOH for 1-8 hours. In order to maintain high levels of O-acetylation on type 5 and/or 8 polysaccharide or oligosaccharide, treatments which would lead to hydrolysis of the O-acetyl groups are minimised. For example treatment at extremes of pH are minimised.

The type 5 and 8 polysaccharides included in the immunogenic composition of the invention are optionally conjugated to a carrier protein as described below or are alternatively unconjugated.

The immunogenic compositions of the invention alternatively contains either type 5 or type 8 polysaccharide.

S. aureus 336 Antigen

In an embodiment, the immunogenic composition of the invention comprises the S. aureus 336 antigen described in U.S. Pat. No. 6,294,177.

The 336 antigen comprises β-linked hexosamine, contains no O-acetyl groups and specifically binds to antibodies to S. aureus Type 336 deposited under ATCC 55804.

In an embodiment, the 336 antigen is a polysaccharide which is of native size or alternatively may be sized, for instance by microfluidisation, ultrasonic irradiation or by chemical treatment. The invention also covers oligosaccharides derived from the 336 antigen.

The 336 antigen, where included in the immunogenic composition of the invention is optionally conjugated to a carrier protein as described below or are alternatively unconjugated.

Type I, II and III Polysaccharides from S. epidermidis

Strains ATCC-31432, SE-360 and SE-10 of S. epidermidis are characteristic of three different capsular types, I, II and III respectively (Ichiman and Yoshida 1981, J. Appl. Bacteriol. 51; 229). Capsular polysaccharides extracted from each serotype of S. epidermidis constitute Type I, II and III polysaccharides. Polysaccharides may be extracted by serval methods including the method described in U.S. Pat. No. 4,197,290 or as described in Ichiman et al 1991, J. Appl. Bacteriol. 71; 176.

In one embodiment of the invention, the immunogenic composition comprises type I and/or II and/or III polysaccharides or oligosaccharides from S. epidermidis.

Polysaccharides are of native size or alternatively may be sized, for instance by microfluidisation, ultrasonic irradiation or chemical cleavage. The invention also covers oligosaccharides extracted from S. epidermidis strains.

These polysaccharides are unconjugated or are optionally conjugated as described below.

Conjugation of Polysaccharides

Amongst the problems associated with the use of polysaccharides in vaccination, is the fact that polysaccharides per se are poor immunogens. Strategies, which have been designed to overcome this lack of immunogenicity, include the linking of the polysaccharide to large protein carriers, which provide bystander T-cell help. In an embodiment, the polysaccharides utilised in the invention are linked to a protein carrier which provide bystander T-cell help. Examples of these carriers which may be used for coupling to polysaccharide or oligosaccharide immunogens include the Diphtheria and Tetanus toxoids (DT, DT Crm197 and TT), Keyhole Limpet Haemocyanin (KLH), Pseudomonas aeruginosa exoprotein A (rEPA) and the purified protein derivative of Tuberculin (PPD), protein D from Haemophilus influenzae, pneumolysin or fragments of any of the above. Fragments suitable for use include fragments encompassing T-helper epitopes. In particular protein D fragment will optionally contain the N-terminal ⅓ of the protein. Protein D is an IgD-binding protein from Haemophilus influenzae (EP 0 594 610 B1).

In an embodiment, a carrier protein used in the immunogenic compositions of the invention comprises or consists of the fragment of a staphylococcal Isd protein, the fragment of a staphylococcal extracellular component binding protein or a fusion protein of the invention as described above.

In an embodiment, EsxA, EsxB, EsaC or EsaB are present in the immunogenic composition of the invention as unconjugated or free proteins (WO 08/19162, WO 10/14304).

The polysaccharides may be linked to the carrier protein(s) by any known method (for example, by Likhite, U.S. Pat. No. 4,372,945 by Armor et al., U.S. Pat. No. 4,474,757, WO and Jennings et al., U.S. Pat. No. 4,356,170). Optionally, CDAP conjugation chemistry is carried out (see WO95/08348).

In CDAP, the cyanylating reagent 1-cyano-dimethylaminopyridinium tetrafluoroborate (CDAP) is optionally used for the synthesis of polysaccharide-protein conjugates. The cyanilation reaction can be performed under relatively mild conditions, which avoids hydrolysis of the alkaline sensitive polysaccharides. This synthesis allows direct coupling to a carrier protein.

The polysaccharide may be solubilized in water or a saline solution. CDAP may be dissolved in acetonitrile and added immediately to the polysaccharide solution. The CDAP reacts with the hydroxyl groups of the polysaccharide to form a cyanate ester. After the activation step, the carrier protein is added. Amino groups of lysine react with the activated polysaccharide to form an isourea covalent link. After the coupling reaction, a large excess of glycine is then added to quench residual activated functional groups. The product is then passed through a gel permeation column to remove unreacted carrier protein and residual reagents.

In an embodiment, the immunogenic composition of the invention comprises a further staphylococcal protein which is optionally a S. aureus or S. epidermidis protein. In an embodiment, the immunogenic composition of the invention further comprises one or more of the proteins described in WO 06/32475 optionally with the sequences described therein (incorporated by reference) or immunogenic fragments thereof. Many of the proteins fall into the categories of extracellular component binding proteins, transporter proteins or toxins and regulators of virulence. The immunogenic composition of the invention optionally further comprises a staphylococcal extracellular component binding protein or a staphylococcal transporter protein or a staphylococcal toxin or regulator of virulence. The immunogenic composition of the invention optionally comprises at least or exactly 1, 2, 3, 4, 5 or 6 staphylococcal proteins.

Preferred immunogenic compositions of the invention comprise a plurality of proteins selected from at least two different categories of protein, having different functions within Staphylococci. Examples of such categories of proteins are extracellular binding proteins, transporter proteins such as Fe acquisition proteins, toxins or regulators of virulence and other immunodominant proteins.

In a preferred embodiment, immunogenic composition of the invention further comprises a number of proteins equal to or greater than 2, 3, 4, 5 or 6 selected from 2, 3 or 4 different groups selected from;
Group a) extracelular component binding proteins;
Group b) transporter proteins;
Group c) toxins or regulators of virulence
Group d) structural proteins.

In a preferred embodiment, immunogenic composition of the invention further comprises a number of proteins equal to or greater than 2, 3, 4, 5 or 6 selected from 2, 3 or 4 of the following groups:
group a)—at least one staphylococcal extracellular component binding protein or fragment thereof selected from the group consisting of laminin receptor, SitC/MntC/saliva binding protein, EbhA, EbhB, Elastin binding protein (EbpS), EFB (FIB), SBI, ClfA, SdrC, SdrD, SdrE, SdrG, SdrH, SasF, lipase GehD, SasA, SasB, SasC, SasD, SasK, FnbA, FnbB, Cna, ClfB, FbpA, Npase, IsaA/PisA, SsaA, EPB, SSP-1, SSP-2, HBP, Vitronectin binding protein, fibrinogen binding protein, coagulase, Fig and MAP;
group b)—at least one staphylococcal transporter protein or fragment thereof selected from the group consisting of Immunodominant ABC transporter, IsdA, IsdB, IsdC, Mg2+ transporter, HarA, SitC and Ni ABC transporter;
group c)—at least one staphylococcal regulator of virulence, toxin or fragment thereof selected from the group consisting of EsaC, EsxA, EsxB, RNA III activating protein (RAP), EsaB;
group d)—at least one staphylococcal structural protein or immunogenic fragment thereof selected from the group consisting of MRPII and autolysin.

Optional combinations to be present in the immunogenic compositon of the invention include IsdA, IsdB and EsaC; SdrC, IsdA and EsaC; IsdA and EsxA; IsdB and EsxA; IsdA, IsdB and EsxA; SdrC, IsdA and EsxA; ClfA, IsdA and EsxB; IsdB and EsxB; IsdA, IsdB and EsxB; SdrC, IsdA and EsxB; SdrD, IsdA and IsdB; SdrC, IsdA and IsdB; SdrE, IsdA and IsdB; SdrG, IsdA and IsdB; IsdA and IsdB; ClfB, IsdA and IsdB; EsaC and IsdA; EsaC and IsdB; EsaC and EsxA; EsaC and EsxB; EsaC and SdrC.

A further aspect of the invention is a polynucleotide having a polynucleotide sequence encoding a NEAT domain of a staphylococcal Isd protein and a polynucleotide sequence encoding a ligand binding domain of a staphylococcal extracellular component binding protein. In an embodiment, the polynucleotide has a sequence encoding a polypeptide having at least 85%, 90%, 95%, 96%, 97%, 98%, 99% or 100% identity to SEQ ID NO:2, 3, 4, 5, 7, 8, 9, 10, 11, 12, 13, 15, 17, 18, 19, 21, 22, 23, 24, 26, 27, 28, 30, 31, 33, 34, 36, 37, 39, 40, 42, 43, 45 or 46.

A further aspect of the invention is a vaccine comprising the immunogenic composition of the invention, the fusion protein of the invention or the polynucleotide of the invention and a pharmaceutically acceptable excipient.

The vaccines of the present invention may be adjuvanted, particularly when intended for use in an elderly population but also for use in infant populations. Suitable adjuvants include an aluminum salt such as aluminum hydroxide gel or aluminum phosphate or alum, but may also be other metal salts such as those of calcium, magnesium, iron or zinc, or may be an insoluble suspension of acylated tyrosine, or acylated sugars, cationically or anionically derivatized saccharides, or polyphosphazenes.

It is preferred that the adjuvant be selected to be a preferential inducer of a TH1 type of response. Such high levels of Th1-type cytokines tend to favour the induction of cell mediated immune responses to a given antigen, whilst high levels of Th2-type cytokines tend to favour the induction of humoral immune responses to the antigen.

The distinction of Th1 and Th2-type immune response is not absolute. In reality an individual will support an immune response which is described as being predominantly Th1 or predominantly Th2. However, it is often convenient to consider the families of cytokines in terms of that described in murine CD4 +ve T cell clones by Mosmann and Coffman (Mosmann, T. R. and Coffman, R. L. (1989) TH1 and TH2 cells: different patterns of lymphokine secretion lead to different functional properties. (Annual Review of Immunology, 7, p 145-173). Traditionally, Th1-type responses are associated with the production of the INF-γ and IL-2 cytokines by T-lymphocytes. Other cytokines often directly associated with the induction of Th1-type immune responses are not produced by T-cells, such as IL-12. In contrast, Th2-type responses are associated with the secretion of Il-4, IL-5, IL-6, IL-10. Suitable adjuvant systems which promote a predominantly Th1 response include: Monophosphoryl lipid A or a derivative thereof (or detoxified lipid A in general—see for instance WO2005107798), particularly 3-de-O-acylated monophosphoryl lipid A (3D-MPL) (for its preparation see GB 2220211 A); and a combination of monophosphoryl lipid A, preferably 3-de-O-acylated monophosphoryl lipid A, together with either an aluminum salt (for instance aluminum phosphate or aluminum hydroxide) or an oil-in-water emulsion. In such combinations, antigen and 3D-MPL are contained in the same particulate structures, allowing for more efficient delivery of antigenic and immunostimulatory signals. Studies have shown that 3D-MPL is able to further enhance the immunogenicity of an alum-adsorbed antigen [Thoelen et al. Vaccine (1998) 16:708-14; EP 689454-B1].

An enhanced system involves the combination of a monophosphoryl lipid A and a saponin derivative, particularly the combination of QS21 and 3D-MPL as disclosed in WO 94/00153, or a less reactogenic composition where the QS21 is quenched with cholesterol as disclosed in WO 96/33739. A particularly potent adjuvant formulation involving QS21, 3D-MPL and tocopherol in an oil in water emulsion is described in WO 95/17210. In one embodiment the immunogenic composition additionally comprises a saponin, which may be QS21. The formulation may also comprise an oil in water emulsion and tocopherol (WO 95/17210). Unmethylated CpG containing oligonucleotides (WO 96/02555) and other immunomodulatory oligonucleotides (WO0226757 and WO03507822) are also preferential inducers of a TH1 response and are suitable for use in the present invention.

Particular adjuvants are those selected from the group of metal Salts, oil in water emulsions, Toll like receptors agonist, (in particular Toll like receptor 2 agonist, Toll like receptor 3 agonist, Toll like receptor 4 agonist, Toll like receptor 7 agonist, Toll like receptor 8 agonist and Toll like receptor 9 agonist), saponins or combinations thereof.

An adjuvant that can be used with the vaccine compositions of the invention are bleb or outer membrane vesicle preparations from Gram negative bacterial strains such as those taught by WO02/09746—particularly N. meningitidis blebs. Adjuvant properties of blebs can be improved by retaining LOS (lipooligosacccharide) on its surface (e.g. through extraction with low concentrations of detergent [for instanct 0-0.1% deoxycholate]). LOS can be detoxified through the msbB(−) or htrB(−) mutations discussed in WO02/09746. Adjuvant properties can also be improved by retaining PorB (and optionally removing PorA) from meningococcal blebs. Adjuvant properties can also be improved by truncating the outer core saccharide structure of LOS on meningococcal blebs—for instance via the IgtB(−) mutation discussed in WO2004/014417. Alternatively, the aforementioned LOS (e.g. isolated from a msbB(−) and/or IgtB(−) strain) can be purified and used as an adjuvant in the compositions of the invention.

A further adjuvant which may be used with the compositions of the invention may be selected from the group: a saponin, lipid A or a derivative thereof, an immunostimulatory oligonucleotide, an alkyl glucosaminide phosphate, an oil in water emulsion or combinations thereof. A further preferred adjuvant is a metal salt in combination with another adjuvant. It is preferred that the adjuvant is a Toll like receptor agonist in particular an agonist of a Toll like receptor 2, 3, 4, 7, 8 or 9, or a saponin, in particular Qs21. It is further preferred that the adjuvant system comprises two or more adjuvants from the above list. In particular the combinations preferably contain a saponin (in particular Qs21) adjuvant and/or a Toll like receptor 9 agonist such as a CpG containing immunostimulatory oligonucleotide. Other preferred combinations comprise a saponin (in particular QS21) and a Toll like receptor 4 agonist such as monophosphoryl lipid A or its 3 deacylated derivative, 3 D—MPL, or a saponin (in particular QS21) and a Toll like receptor 4 ligand such as an alkyl glucosaminide phosphate.

Particularly preferred adjuvants are combinations of 3D-MPL and QS21 (EP 0 671 948 B1), oil in water emulsions comprising 3D-MPL and QS21 (WO 95/17210, WO 98/56414), or 3D-MPL formulated with other carriers (EP 0 689 454 B1). Other preferred adjuvant systems comprise a combination of 3 D MPL, QS21 and a CpG oligonucleotide as described in U.S. Pat. No. 6,558,670, U.S. Pat. No. 6,544,518.

The adjuvant may be an oil in water emulsion or may comprise an oil in water emulsion in combination with other adjuvants. The oil phase of the emulsion system preferably comprises a metabolisable oil. The meaning of the term metabolisable oil is well known in the art. Metabolisable can be defined as “being capable of being transformed by metabolism” (Dorland's Illustrated Medical Dictionary, W.B. Sanders Company, 25^{th} edition (1974)). The oil may be any vegetable oil, fish, oil, animal or synthetic oil, which is not toxic to the recipient and is capable of being transformed by metabolism. Nuts, seeds, and grains are common sources of vegetable oils. Synthetic oils are also part of this invention and can include commercially available oils such as NEOBEE® and others. Squalene (2,6,10,15,19,23-Hexamethyl-2,6,10,14,18,22-tetracosahexaene) is an unsaturated oil which is found in large quantities in shark-liver oil, and in lower quantities in olive oil, wheat germ oil, rice bran oil, and yeast, and is a particularly preferred oil for use in this invention. Squalene is a metabolisable oil by virtue of the fact that it is an intermediate in the biosynthesis of cholesterol (Merck index, 10^{th} Edition, entry no. 8619).

Tocols (e.g. vitamin E) are also often used in oil emulsions adjuvants (EP 0 382 271 B1; U.S. Pat. No. 5,667,784; WO 95/17210). Tocols used in the oil emulsions (preferably oil in water emulsions) of the invention may be formulated as described in EP 0 382 271 B1, in that the tocols may be dispersions of tocol droplets, optionally comprising an emulsifier, of preferably less than 1 micron in diameter. Alternatively, the tocols may be used in combination with another oil, to form the oil phase of an oil emulsion. Examples of oil emulsions which may be used in combination with the tocol are described herein, such as the metabolisable oils described above.

Oil in water emulsion adjuvants per se have been suggested to be useful as adjuvant compositions (EP 0 399 843B), also combinations of oil in water emulsions and other active agents have been described as adjuvants for vaccines (WO 95/17210; WO 98/56414; WO 99/12565; WO 99/11241). Other oil emulsion adjuvants have been described, such as water in oil emulsions (U.S. Pat. No. 5,422,109; EP 0 480 982 B2) and water in oil in water emulsions (U.S. Pat. No. 5,424,067; EP 0 480 981 B). All of which form preferred oil emulsion systems (in particular when incorporating tocols) to form adjuvants and compositions of the present invention.

Most preferably the oil emulsion (for instance oil in water emulsions) further comprises an emulsifier such as TWEEN 80 and/or a sterol such as cholesterol.

A preferred oil emulsion (preferably oil-in-water emulsion) comprises a metabolisible, non-toxic oil, such as squalane, squalene or a tocopherol such as alpha tocopherol (and preferably both squalene and alpha tocopherol) and optionally an emulsifier (or surfactant) such as Tween 80. A sterol (preferably cholesterol) may also be included.

The method of producing oil in water emulsions is well known to the man skilled in the art. Commonly, the method comprises mixing the tocol-containing oil phase with a surfactant such as a PBS/TWEEN80™ solution, followed by homogenisation using a homogenizer, it would be clear to a man skilled in the art that a method comprising passing the mixture twice through a syringe needle would be suitable for homogenising small volumes of liquid. Equally, the emulsification process in microfluidiser (M110S Microfluidics machine, maximum of 50 passes, for a period of 2 minutes at maximum pressure input of 6 bar (output pressure of about 850 bar)) could be adapted by the man skilled in the art to produce smaller or larger volumes of emulsion. The adaptation could be achieved by routine experimentation comprising the measurement of the resultant emulsion until a preparation was achieved with oil droplets of the required diameter.

In an oil in water emulsion, the oil and emulsifier should be in an aqueous carrier. The aqueous carrier may be, for example, phosphate buffered saline.

The size of the oil droplets found within the stable oil in water emulsion are preferably less than 1 micron, may be in the range of substantially 30-600 nm, preferably substantially around 30-500 nm in diameter, and most preferably substantially 150-500 nm in diameter, and in particular about 150 nm in diameter as measured by photon correlation spectroscopy. In this regard, 80% of the oil droplets by number should be within the preferred ranges, more preferably more than 90% and most preferably more than 95% of the oil droplets by number are within the defined size ranges. The amounts of the components present in the oil emulsions of the present invention are conventionally in the range of from 0.5-20% or 2 to 10% oil (of the total dose volume), such as squalene; and when present, from 2 to 10% alpha tocopherol; and from 0.3 to 3% surfactant, such as polyoxyethylene sorbitan monooleate. Preferably the ratio of oil (preferably squalene):tocol (preferably α-tocopherol) is equal or less than 1 as this provides a more stable emulsion. An emulsifier, such as Tween80 or Span 85 may also be present at a level of about 1%. In some cases it may be advantageous that the vaccines of the present invention will further contain a stabiliser.

Examples of preferred emulsion systems are described in WO 95/17210, WO 99/11241 and WO 99/12565 which disclose emulsion adjuvants based on squalene, α-tocopherol, and TWEEN 80, optionally formulated with the immunostimulants QS21 and/or 3D-MPL. Thus in a particularly, preferred embodiment of the present invention, the adjuvant of the invention may additionally comprise further immunostimulants, such as LPS or derivatives thereof, and/or saponins. Examples of further immunostimulants are described herein and in “Vaccine Design—The Subunit and Adjuvant Approach” 1995, Pharmaceutical Biotechnology, Volume 6, Eds. Powell, M. F., and Newman, M. J., Plenum Press, New York and London, ISBN 0-306-44867-X.

In a preferred aspect the adjuvant and immunogenic compositions according to the invention comprise a saponin (preferably QS21) and/or an LPS derivative (preferably 3D-MPL) in an oil emulsion described above, optionally with a sterol (preferably cholesterol). Additionally the oil emulsion (preferably oil in water emulsion) may contain span 85 and/or lecithin and/or tricaprylin. Adjuvants comprising an oil-in-water emulsion, a sterol and a saponin are described in WO 99/12565.

Typically for human administration the saponin (preferably QS21) and/or LPS derivative (preferably 3D-MPL) will be present in a human dose of immunogenic composition in the range of 1 μg-200 μg, such as 10-100 μg, preferably 10 μg-50 μg per dose. Typically the oil emulsion (preferably oil in water emulsion) will comprise from 2 to 10% metabolisible oil. Preferably it will comprise from 2 to 10% squalene, from 2 to 10% alpha tocopherol and from 0.3 to 3% (preferably 0.4-2%) emulsifier (preferably tween 80 [polyoxyethylene sorbitan monooleate]). Where both squalene and alpha tocopherol are present, preferably the ratio of squalene:alpha tocopherol is equal to or less than 1 as this provides a more stable emulsion. Span 85 (Sorbitan trioleate) may also be present at a level of 0.5 to 1% in the emulsions used in the invention. In some cases it may be advantageous that the immunogenic compositions and vaccines of the present invention will further contain a stabiliser, for example other emulsifiers/surfactants, including caprylic acid (merck index 10^{th} Edition, entry no. 1739), of which Tricaprylin is particularly preferred.

Where squalene and a saponin (preferably QS21) are included, it is of benefit to also include a sterol (preferably cholesterol) to the formulation as this allows a reduction in the total level of oil in the emulsion. This leads to a reduced cost of manufacture, improvement of the overall comfort of the vaccination, and also qualitative and quantitative improvements of the resultant immune responses, such as improved IFN-γ production. Accordingly, the adjuvant system of the present invention typically comprises a ratio of metabolisable oil:saponin (w/w) in the range of 200:1 to 300:1, also the present invention can be used in a “low oil” form the preferred range of which is 1:1 to 200:1, preferably 20:1 to 100:1, and most preferably substantially 48:1, this vaccine retains the beneficial adjuvant properties of all of the components, with a much reduced reactogenicity profile. Accordingly, the particularly preferred embodiments have a ratio of squalene:QS21 (w/w) in the range of 1:1 to 250:1, also a preferred range is 20:1 to 200:1, preferably 20:1 to 100:1, and most preferably substantially 48:1. Preferably a sterol (most preferably cholesterol) is also included present at a ratio of saponin:sterol as described herein.

The emulsion systems of the present invention preferably have a small oil droplet size in the sub-micron range. Most preferably the oil droplet sizes will be in the range 120 to 750 nm, and most preferably from 120-600 nm in diameter.

A particularly potent adjuvant formulation (for ultimate combination with AIPO4 in the immunogenic compositions of the invention) involves a saponin (preferably QS21), an LPS derivative (preferably 3D-MPL) and an oil emulsion (preferably squalene and alpha tocopherol in an oil in water emulsion) as described in WO 95/17210 or in WO 99/12565 (in particular adjuvant formulation 11 in Example 2, Table 1).

Examples of a TLR 2 agonist include peptidoglycan or lipoprotein. Imidazoquinolines, such as Imiquimod and Resiquimod are known TLR7 agonists. Single stranded RNA is also a known TLR agonist (TLR8 in humans and TLR7 in mice), whereas double stranded RNA and poly IC (polyinosinic-polycytidylic acid—a commercial synthetic mimetic of viral RNA). are exemplary of TLR 3 agonists. 3D-MPL is an example of a TLR4 agonist whilst CPG is an example of a TLR9 agonist.

The immunogenic composition may comprise an antigen and an immunostimulant adsorbed onto a metal salt. Aluminium based vaccine formulations wherein the antigen and the immunostimulant 3-de-O-acylated monophosphoryl lipid A (3D-MPL), are adsorbed onto the same particle are described in EP 0 576 478 B1, EP 0 689 454 B1, and EP 0 633 784 B1. In these cases then antigen is first adsorbed onto the aluminium salt followed by the adsorption of the immunostimulant 3D-MPL onto the same aluminium salt particles. Such processes first involve the suspension of 3D-MPL by sonication in a water bath until the particles reach a size of between 80 and 500 nm. The antigen is typically adsorbed onto aluminium salt for one hour at room temperature under agitation. The 3D-MPL suspension is then added to the adsorbed antigen and the formulation is incubated at room temperature for 1 hour, and then kept at 4° C. until use.

The vaccine preparations of the present invention may be used to protect or treat a mammal susceptible to infection, by means of administering said vaccine via systemic or mucosal route. These administrations may include injection via the intramuscular, intraperitoneal, intradermal or subcutaneous routes; or via mucosal administration to the oral/alimentary, respiratory, genitourinary tracts. Intranasal administration of vaccines for the treatment of pneumonia or otitis media is preferred (as nasopharyngeal carriage of pneumococci can be more effectively prevented, thus attenuating infection at its earliest stage). Although the vaccine of the invention may be administered as a single dose, components thereof may also be co-administered together at the same time or at different times (for instance pneumococcal polysaccharides could be administered separately, at the same time or 1-2 weeks after the administration of any bacterial protein component of the vaccine for optimal coordination of the immune responses with respect to each other). For co-administration, the optional Th1 adjuvant may be present in any or all of the different administrations, for example, it may be present in combination with the bacterial protein component of the vaccine. In addition to a single route of administration, 2 different routes of administration may be used. For example, polysaccharides may be administered IM (or ID) and bacterial proteins may be administered IN (or ID). In addition, the vaccines of the invention may be administered IM for priming doses and IN for booster doses.

The amount of conjugate antigen in each vaccine dose is selected as an amount which induces an immunoprotective response without significant, adverse side effects in typical vaccines. Such amount will vary depending upon which specific immunogen is employed and how it is presented. Generally, it is expected that each dose will comprise 0.1-100 μg of polysaccharide, typically 0.1-50 μg, 0.1-10 μg, 1-10 μg or 1-5 μg for polysaccharide conjugates.

The content of protein antigens in the vaccine will typically be in the range 1-100 μg, 5-50 μg or 5-25 μg. Following an initial vaccination, subjects may receive one or several booster immunizations adequately spaced.

Vaccine preparation is generally described in Vaccine Design (“The subunit and adjuvant approach” (eds Powell M. F. & Newman M. J.) (1995) Plenum Press New York). Encapsulation within liposomes is described by Fullerton, U.S. Pat. No. 4,235,877.

The vaccines of the present invention may be stored in solution or lyophilized. Optionally the solution is lyophilized in the presence of a sugar such as sucrose, trehalose or lactose. It is typical that they are lyophilized and extemporaneously reconstituted prior to use. Lyophilizing may result in a more stable composition (vaccine).

A further aspect of the invention is a process for making the vaccine of the invention comprising the step of adding a pharmaceutically acceptible excipient to the immunogenic composition, NEAT domain or other fragment, the fusion protein or the polynucleotide of the invention.

The invention also encompasses method of treatment or staphylococcal infection, particularly hospital acquired nosocomial infections.

This immunogenic composition or vaccine of the invention is particularly advantageous to use in cases of elective surgery. Such patients will know the date of surgery in advance and could be inoculated in advance. Since it is not know whether the patient will be exposed to S. aureus or S. epidermidis infection, it is preferred to inoculate with a vaccine of the invention that protects against both, as described above. Typically adults over 16 awaiting elective surgery are treated with the immunogenic compositions and vaccines of the invention. Alternatively children aged 3-16 awaiting elective surgery are treated with the immunogenic compositions and vaccines of the invention.

It is also possible to inoculate health care workers with the vaccine of the invention.

The vaccine preparations of the present invention may be used to protect or treat a mammal susceptible to infection, by means of administering said vaccine via systemic or mucosal route. These administrations may include injection via the intramuscular, intraperitoneal, intradermal or subcutaneous routes; or via mucosal administration to the oral/alimentary, respiratory, genitourinary tracts.

The amount of antigen in each vaccine dose is selected as an amount which induces an immunoprotective response without significant, adverse side effects in typical vaccines. Such amount will vary depending upon which specific immunogen is employed and how it is presented. The protein content of the vaccine will typically be in the range 1-100 μg, 5-50 μg, typically in the range 10-25 μg. An optimal amount for a particular vaccine can be ascertained by standard studies involving observation of appropriate immune responses in subjects. Following an initial vaccination, subjects may receive one or several booster immunisations adequately spaced.

Although the vaccines of the present invention may be administered by any route, administration of the described vaccines into the skin (ID) forms one embodiment of the present invention. Human skin comprises an outer “horny” cuticle, called the stratum corneum, which overlays the epidermis. Underneath this epidermis is a layer called the dermis, which in turn overlays the subcutaneous tissue. Researchers have shown that injection of a vaccine into the skin, and in particular the dermis, stimulates an immune response, which may also be associated with a number of additional advantages. Intradermal vaccination with the vaccines described herein forms an optional feature of the present invention.

The conventional technique of intradermal injection, the “mantoux procedure”, comprises steps of cleaning the skin, and then stretching with one hand, and with the bevel of a narrow gauge needle (26-31 gauge) facing upwards the needle is inserted at an angle of between 10-15°. Once the bevel of the needle is inserted, the barrel of the needle is lowered and further advanced whilst providing a slight pressure to elevate it under the skin. The liquid is then injected very slowly thereby forming a bleb or bump on the skin surface, followed by slow withdrawal of the needle.

More recently, devices that are specifically designed to administer liquid agents into or across the skin have been described, for example the devices described in WO 99/34850 and EP 1092444, also the jet injection devices described for example in WO 01/13977; U.S. Pat. No. 5,480,381, U.S. Pat. No. 5,599,302, U.S. Pat. No. 5,334,144, U.S. Pat. No. 5,993,412, U.S. Pat. No. 5,649,912, U.S. Pat. No. 5,569,189, U.S. Pat. No. 5,704,911, U.S. Pat. No. 5,383,851, U.S. Pat. No. 5,893,397, U.S. Pat. No. 5,466,220, U.S. Pat. No. 5,339,163, U.S. Pat. No. 5,312,335, U.S. Pat. No. 5,503,627, U.S. Pat. No. 5,064,413, U.S. Pat. No. 5,520,639, U.S. Pat. No. 4,596,556, U.S. Pat. No. 4,790,824, U.S. Pat. No. 4,941,880, U.S. Pat. No. 4,940,460, WO 97/37705 and WO 97/13537. Alternative methods of intradermal administration of the vaccine preparations may include conventional syringes and needles, or devices designed for ballistic delivery of solid vaccines (WO 99/27961), or transdermal patches (WO 97/48440; WO 98/28037); or applied to the surface of the skin (transdermal or transcutaneous delivery WO 98/20734; WO 98/28037).

When the vaccines of the present invention are to be administered to the skin, or more specifically into the dermis, the vaccine is in a low liquid volume, particularly a volume of between about 0.05 ml and 0.2 ml.

The content of antigens in the skin or intradermal vaccines of the present invention may be similar to conventional doses as found in intramuscular vaccines (see above). However, it is a feature of skin or intradermal vaccines that the formulations may be “low dose”. Accordingly the protein antigens in “low dose” vaccines are optionally present in as little as 0.1 to 10 μg, optionally 0.1 to 5 μg per dose; and the polysaccharide (optionally conjugated) antigens may be present in the range of 0.01-1 μg, and optionally between 0.01 to 0.5 μg of polysaccharide per dose.

As used herein, the term “intradermal delivery” means delivery of the vaccine to the region of the dermis in the skin. However, the vaccine will not necessarily be located exclusively in the dermis. The dermis is the layer in the skin located between about 1.0 and about 2.0 mm from the surface in human skin, but there is a certain amount of variation between individuals and in different parts of the body. In general, it can be expected to reach the dermis by going 1.5 mm below the surface of the skin. The dermis is located between the stratum corneum and the epidermis at the surface and the subcutaneous layer below. Depending on the mode of delivery, the vaccine may ultimately be located solely or primarily within the dermis, or it may ultimately be distributed within the epidermis and the dermis.

An embodiment of the invention is a method of preventing or treating staphylococcal infection or disease comprising the step of administering the immunogenic composition or vaccine of the invention to a patient in need thereof.

A further embodiment of the invention is a use of the immunogenic composition of the invention in the manufacture of a vaccine for treatment or prevention of staphylococcal infection or disease, optionally post-surgery staphylococcal infection.

The term ‘staphylococcal infection’ encompasses infection caused by S. aureus and/or S. epidermidis and other staphylococcal strains capable of causing infection in a mammalina, optionally human host.

The terms “comprising”, “comprise” and “comprises” herein are intended by the inventors to be optionally substitutable with the terms “consisting of”, “consist of” and “consists of”, respectively, in every instance.

All references or patent applications cited within this patent specification are incorporated by reference herein.

In order that this invention may be better understood, the following examples are set forth. These examples are for purposes of illustration only, and are not to be construed as limiting the scope of the invention in any manner.

### EXAMPLES

### Example 1

### IsdA/IsdB Constructs and Immunogenicity in Mice

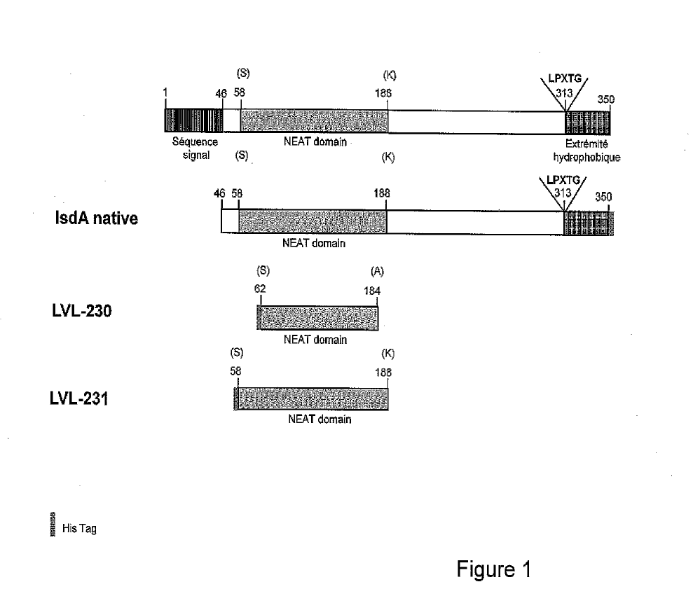

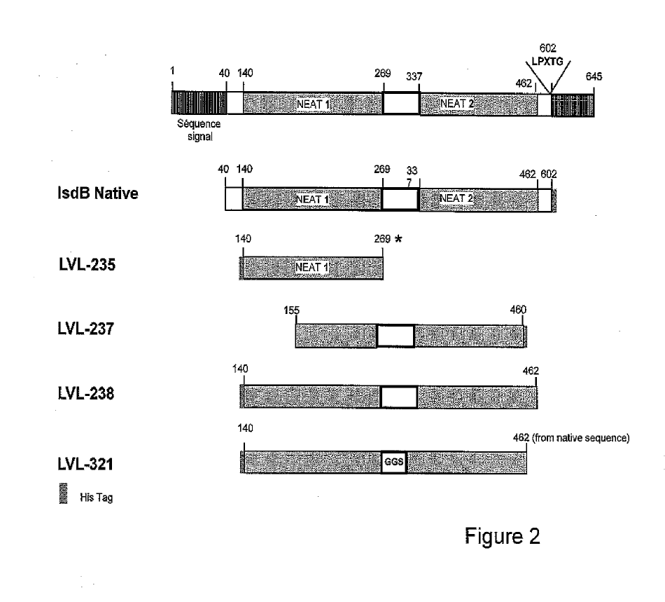

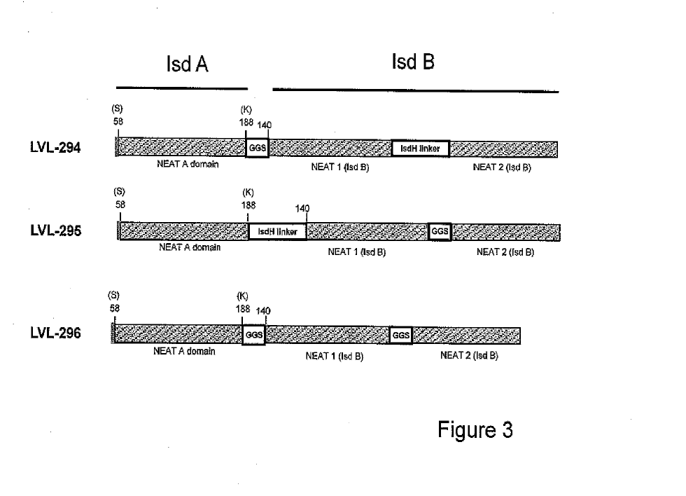

Details of IsdA and IsdB proteins, fragments and fusion proteins using in this example are shown in FIGS. 1-3.

Groups of 15 female Balb/C mice were immunised intramuscularly on days 0, 14 and 28 with 10 μg of each of the following proteins adjuvanted with an adjuvant containing 3D-MPL and QS21 in an oil in water emulsion (AS02V). A control group of 10 mice was immunised with adjuvant alone:

| | |
|---|---|
| Group 1 | full length IsdA protein |
| Group 2 | full length IsdB protein |
| Group 3 | LVL230 IsdA NEAT domain (amino acids 62-184) |
| Group 4 | LVL231 IsdA NEAT domain (amino acids 58-188) |
| Group 5 | LVL235 IsdB NEAT1 domain (amino acids 140-269) |
| Group 6 | LVL237 IsdB NEAT1/2 fusion (amino acids 155-460) |
| Group 7 | LVL238 NEAT 1-NEAT 2 IsdB (amino acids 140-462) |
| Group 8 | LVL294 IsdA NEAT (58-188) - GGS-IsdB NEAT 1 - |
| | IsdH-IsdB NEAT 2 |
| Group 9 | LVL295 IsdA NEAT (58-188)-IsdH-IsdB NEAT 1-GGS - |
| | IsdB NEAT 2 |
| Group 10 | LVL296 IsdA NEAT (58-188)- GGS- IsdB NEAT1 - GGS - |
| | IsdB NEAT2 |
| Group11 | LVL321 IsdB NEAT1 - GGS - IsdB NEAT2 (amino acids |
| | 140-462) |
| Group 12 | adjuvant |

GGS indicates a linker made up of the amino acids glycine, glycine, serine
IsdH indicates the sequence of SEQ ID NO: 98.
Anti-IsdB and anti-IsdA ELISA titers were determined in individual sera collected at day 42 (post III). OPA titers were determined on pooled Post III sera.

Anti-IsdA and Anti-IsdB ELISA Response

Purified IsdA or IsdB were coated at 1 μg/ml in phosphate buffered saline (PBS) on high-binding microtitre plates (Nunc Maxisorp), overnight at 4° C. The plates were blocked with PBS-BSA 1% for 30 min at RT with agitation. The mouse anti-sera were prediluted 1/500 and then, further twofold dilutions were made in microplates and incubated at RT for 30 min with agitation. After washing, bound murine antibody was detected using Jackson ImmunoLaboratories Inc. peroxidase-conjugated affiniPure Goat Anti-Mouse IgG (H+L) (ref: 115-035-003) diluted 1:5000 in PBS-BSA0.2%-tween 0.05%. The detection antibodies were incubated for 30 min. at room temperature with agitation. The color was developed using 4 mg OPD+5 μl H2O2 per 10 ml pH 4.5 0.1M citrate buffer for 15 minutes in the dark at room temperature. The reaction was stopped with 50 μl HCl, and the optical density (OD) was read at 490 nm relative to 620 nm.

The level of anti-IsdA or anti-IsdB antibodies present in the sera was expressed in mid-point titers. A GMT was calculated for the 15 samples (10 for the controls).

Opsonophagocytosis Assay

The osponophagocytosis assay (OPA) was performed in round-bottom microplates with 15 μl of HL-60 phagocytic cells (adjusted to 6.7 10e7/ml), 15 μl of S. aureus bacteria, 15 μl of the test serum dilutions and 15 μl of piglet complement (1% final concentration).

The inactivated test sera were first diluted (1/25) in MEM-BSA 1% HEPES 25 mM and incubated with a S. aureus Newman D spa strain (diluted in order to obtain 300 CFU/well at the end of the test) for 40 min at RT with shaking. Before dilution the strain was previously grown overnight in a iron-deficient medium (RPMI 1640)

The HL-60 cells (adjusted to 6.7·10e7/ml) and the piglet complement were then added in each well. A control with inactivated complement was also included for each test sample. The reaction mixture was incubated at 37° C. for 90 minutes with agitation. After a 1/20 dilution, 50 μl of the volume was then transferred into a flat-bottom microplate. 50 μl of MH agar followed by 50 μl of PBS-0.9% agar were added. Automated colony counts were performed after an overnight incubation at 37° C.

The opsonophagocytic activity was expressed as the reciprocal of the serum dilution giving at least 50% killing.

ELISA Results

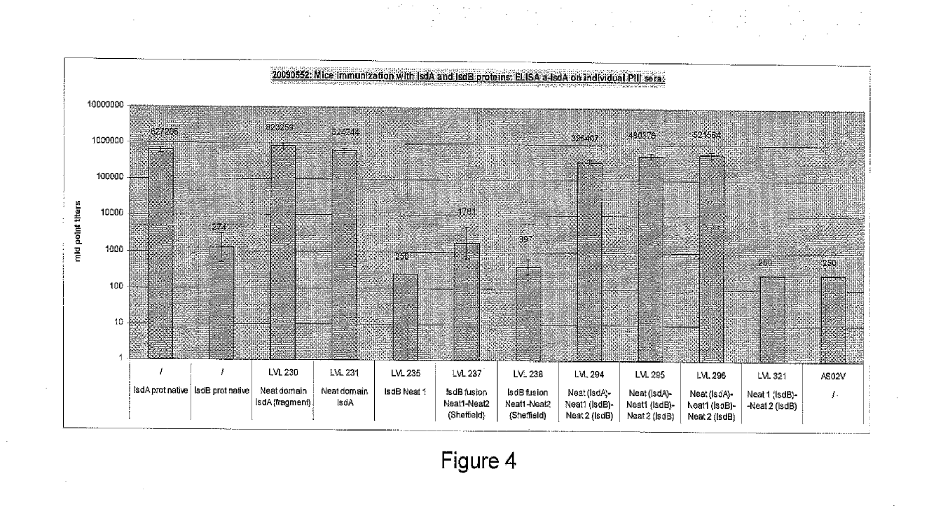

The results shown in FIG. 4 show that the two IsdA NEAT domain fragments in LVL230 and LVL231 both elicited good immune responses against IsdA with ELISA result being as good as those produced against the native protein. Similarly, the three IsdA/IsdB fusion proteins (LVL294, LVL295 and LVL296) gave anti-IsdA responses which were not significantly different from that achieved by full length IsdA.

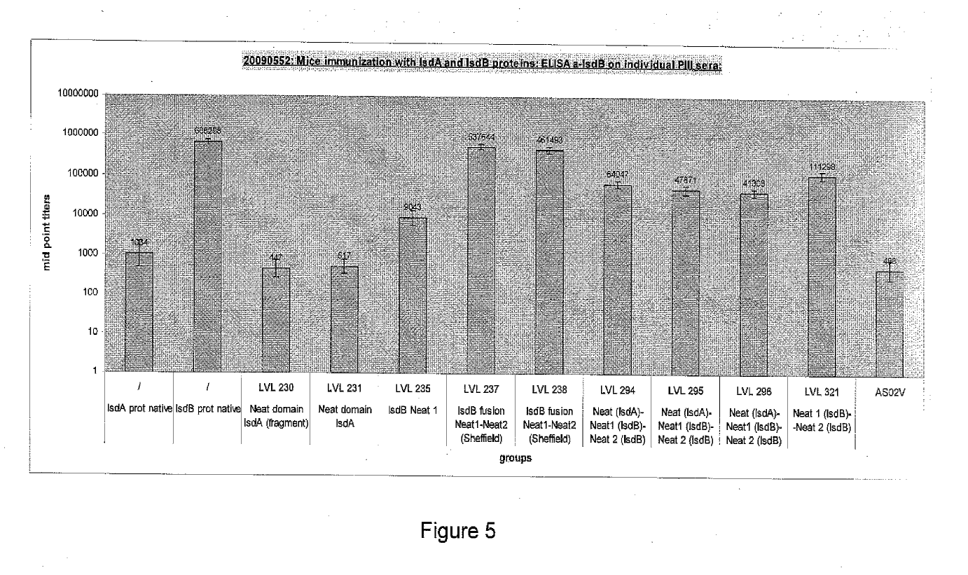

The results shown in FIG. 5 demonstate the importance of the second NEAT domain in the immune response to IsdB. The N-terminal NEAT domain present in LVL235 generated only a small ELISA response (midpoint titre of 9,043) compared to the immune response generated to LVL237 which contained only part of the N-terminal NEAT domain but all of the C-terminal NEAT domain (mid point titre 537,644). This is demonstrated by the response to LVL235 being lower than that obtained with LVL237, LVL238, LVL294, LVL295, LVL296 or LVL321.

Opsono Results

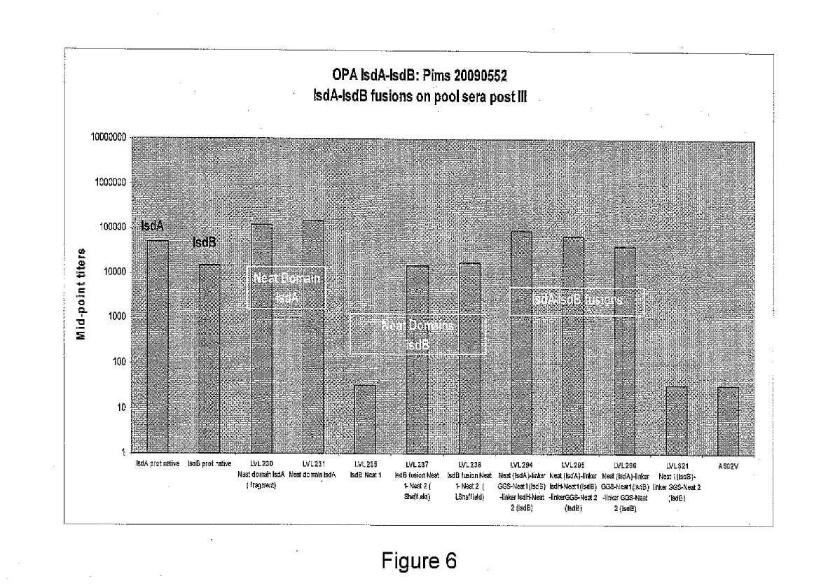

The results achieved in opsono assays are shown in FIG. 6. Very good results were achieved after immunisation with IsdA Neat domain fragments with both LVL230 and LVL231 gaving very good opsono titres; higher than that achieved by full length IsdA or IsdB. These results also confirmed the importance of the C-terminal NEAT domain of IsdB since the N-terminal Neat domain of IsdB (LVL235) generated a lower opsono response compared to LVL237 which contains only part of the N-terminal NEAT domain and all of the C-terminal NEAT domain. Fusion proteins containing a combination of IsdA Neat domain and IsdB Neat domains 1 and 2 gave even higher opsono responses (LVL294, LVL295, LVL296). The presence of SEQ ID NO:98 as a linker between the two IsdB NEAT domains gave in LVL294, provided a particularly good opsono response.

Conclusion

IsdA NEAT domains from amino acids 62-184 and 58-188 both gave excellent immunogenicity results, either as free domains or as part of a fusion protein. Anti-IsdA antibodies also gave a higher Opsono result than anti-IsdB antibodies.

The C-terminal NEAT domain of IsdB is more important thatn the N-terminal NEAT domain for the generation of immunogenicity, particular opsono activity.

### Example 2

### Expression and Purification of ClfA N123 Domains

B2312:

The clfA gene fragment from Staphylococcus aureus NCTC8325 strain coding for amino acids 40 to 559 was codon-optimized and synthesized in 2 portions by GeneArt (Regensburg, Germany). This gene fragment encodes for three structural domains identified as N1, N2 and N3 which contains the fibrinogen-binding activity of ClfA. To enable ligation, the restriction sites NdeI and SacII were added at the extremities of the first synthetic gene portion, while SacII and XhoI were added to the second.

PCR reaction was used to add stop codons at its 3′ end just before the XhoI site and the tyrosine residue at position 474 was replaced by a histidine residue in the second synthetic fragment. The 2 fragments were thus cloned into the pET24b (+) expression vector (Novagen) using the rapid DNA ligation kit (Roche, Mannheim, Germany) by which the DNA fragments and the plasmid were assembled simultaneously. Finally, the final construct was generated following transformation of E. coli strain BLR (DE3) with the expression vector containing the N123 domain (with mut474) standard procedures.

E. coli BLR (DE3) strain: F⁻ ompT hsdS_{B}(r_{B}⁻m_{B}⁻) gal dcm (DE3) □(srl-recA)306::Tn10 (Tet^{R}). (Novagen)

BLR is a recA-derivative of BL21 that improves plasmid monomer yields and may help stabilize target plasmids containing repetitive sequences or whose products may cause the loss of the DE3 prophage.

This strain is tetracycline resistant (12.5 μg/ml).

DE3 indicates that the host is a lysogen of DE3, and therefore carries a chromosomal copy of the T7 RNA polymerase gene under control of the lacUV5 promoter. Such strains are suitable for production of protein from target genes cloned in pET vectors by induction with IPTG.

B2378:

The wild-type sequence of N123 domain (amino acids 40-559 without a mutation at 474) was restored by site-directed mutagenesis (Quickchange Site-directed Mutagenesis Kit; Stratagene) using the expression vector containing the N123 mutation (with mut474) as template. The final strain was generated by the transformation of E. coli strain BLR (DE3) with the expression vector containing the N123 domain (wild-type sequence) according standard procedures.

Purification of ClfA Mutated at aa 474 (Strain B2312)

E. coli cell paste harvested from a culture grown in a fermentor was resuspended in 50 mM phosphate buffer pH 7.2 containing 50 mM NaCl, 2 mM EDTA and 1 mM PMSF to reach an OD_{650 nm} of 120. The suspension was submitted to mechanical disruption in a Panda homogeniser (2 passes—750 bars) and adjusted to pH 4.0 with acetic acid 50%. After centrifugation at 12200 g for 30 min at 4° C., the supernatant was clarified on a 0.45-0.20 μm filter and diafiltered against 1 volume Tris 0.5M pH 8.1 followed by 4 volumes Tris 20 mM-NaCl 120 mM pH 8.1. The retentate was loaded onto a CaptoQ column (˜10 ml retentate per ml resin) equilibrated with Tris 20 mM-NaCl 120 mM pH 8.1. After washing with the equilibration buffer, ClfA was eluted with Tris 20 mM-NaCl 215 mM pH 8.1 and further purified on a Sephacryl HR300 column equilibrated and eluted with 10 mM Na borate pH 9.5. The fractions containing ClfA were selected on basis of purity by SDS-PAGE, pooled and sterile-filtered on 0.22 μm.

Purification of Wild-Type ClfA (Strain B2378)

E. coli cell paste harvested from a culture in a shake flask was resuspended in 50 mM phosphate buffer pH 7.2 containing 50 mM NaCl, 2 mM EDTA and 1 mM PMSF to reach an OD_{650 nm} of 120. The suspension was submitted to mechanical disruption in a Panda homogeniser (2 passes—750 bars) and adjusted to pH 3.8 with acetic acid 50%. After centrifugation at 12200 g for 30 min at 4° C., the supernatant was clarified by 0.45-0.22 μm filtration and purified on a Sephacryl HR300 column equilibrated and eluted with 10 mM Na borate pH 9.5. The fractions containing ClfA were selected on basis of purity by SDS-PAGE, pooled and sterile-filtered on 0.22 μm

### Example 3

### Fibrinogen Binding Experiments

Fibrinogen Adhesion to Coated ClfA:

ClfA proteins were coated at 10 μg/ml in phosphate buffered saline (PBS) on high binding microtitre plates (Nunc Maxisorp) overnight at 4° C. The plates were blocked with PBS-BSA 1% for 30 min at room temperature with shaking.

After washing, human fibrinogen (ref: SIGMA F4883-16) was added at a 1 mg/ml starting concentration, then further twofold dilutions were made in microplates which were incubated for 1 hour at 37° C. with shaking.

After washing, the bound fibrinogen was detected using a peroxydase conjugated anti-fibrinogen goat polyclonal antibody (ref: ABCAM 7539-1) diluted 1:5000 in PBS-BSA 0.2%-Tween 0.05%. The detection antibodies were incubated for 60 minutes at room temperature with agitation.

The color was developed using 4 mg OPD (Sigma)+5 μl H2O2 per 10 ml pH 4.5 0.1M citrate buffer for 15 minutes in the dark at room temperature. The reaction was stopped with 50 μl HCl, and the optical density was read at 490 nm relative to 620 nm.

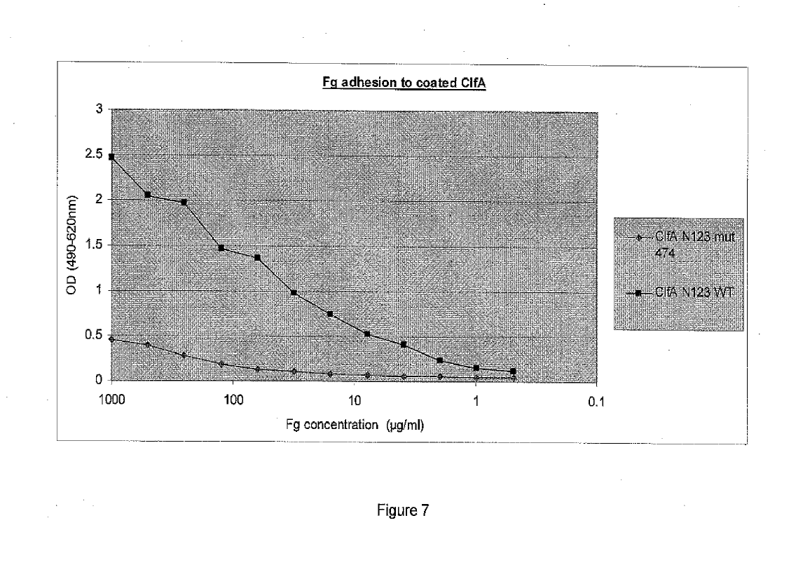

The results are shown in FIG. 7 which shows that the 474 mutant ClfA N123 protein bound poorly to fibrinogen compared to the wild tpe ClfA N123 protein.

ClfA Adhesion to Coated Fibrinogen:

Human fibrinogen (ref: SIGMA F4883-16) was coated at 10 μg/ml in phosphate buffered saline (PBS) on high binding microtitre plates (Nunc Maxisorp) overnight at 4° C. The plates were blocked with PBS-BSA 1% for 30 min at room temperature with shaking.

After washing, the ClfA was added at a 50 μg/ml starting concentration, then further twofold dilutions were made in microplates which were incubated for 1 hour at 37° C. with shaking.

After washing, the bound ClfA was detected using anti-ClfA rabbit polyclonal (obtained after immunization with his-tagged N123 ClfA) diluted 1:500 in PBS-BSA 0.2%-Tween 0.05% and incubated for 1 hour at 37° C. with shaking.

After washing, bound rabbit antibody was detected using Jackson ImmunoLaboratories Inc. peroxidase-conjugated affiniPure Goat Anti-Rabbit IgG (ref: 111-035-003) diluted 1:5000 in PBS-Tween 0.05%. The detection antibodies were incubated for 30 minutes at room temperature with shaking.

The color was developed using 4 mg OPD (Sigma)+5 μl H2O2 per 10 ml pH 4.5 0.1M citrate buffer for 15 minutes in the dark at room temperature. The reaction was stopped with 50 μl HCl, and the optical density was read at 490 nm relative to 620 nm.

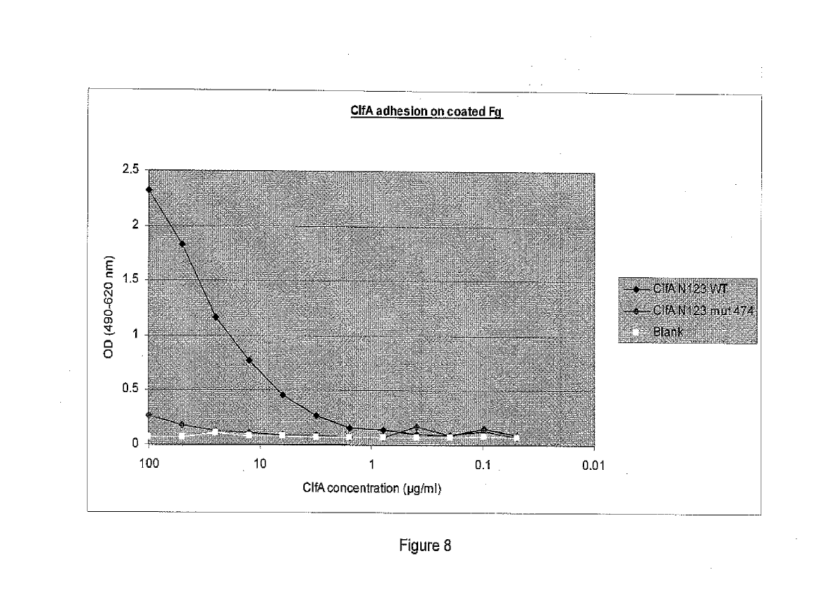

The results shown in FIG. 8 demonstrate again that the 474 mutant ClfA N123 protein bound very poorly to fibrinogen compared to the wild type ClfA N123 protein.

### Example 4

### Inhibition Assay of Fibrinogen Adhesion to Coated ClfA

Groups of 20 mice were inoculated intramuscularly with 10 μg of N123 or mutated 474 ClfA formulated with the adjuvant AS02V, on days 0, 14 and 28. A control group was inoculated with the adjuvant alone.

On day 42 serum was collected from the mice and pooled sera from each group were tested in an inhibition assay of fibrinogen adhesion to coated ClfA.

Purified ClfA was coated at 10 μg/ml in phosphate buffered saline (PBS) on high binding microtitre plates (Nunc Maxisorp) overnight at 4° C. The plates were blocked with PBS-BSA 1% for 30 min at room temperature with agitation. After washing, the mice antisera were added at a 10-fold starting dilution, then further twofold dilutions were made in microplates which were incubated at room temperature for 1 hour with shaking. Without a washing step, human fibrinogen (Ref: SIGMA F4883-16) was added at a 400 μg/ml concentration in PBS-BSA 0.2%-Tween 0.05% and was incubated at 37° C. for 1 hour with shaking.

After washing, the bound fibrinogen was detected using a peroxydase conjugated anti-fibrinogen goat polyclonal antibody (ref: ABCAM 7539-1) diluted 1:5000 in PBS-BSA 0.2%-Tween 0.05%. The detection antibodies were incubated for 60 minutes at room temperature with agitation. The color was developed using 4 mg OPD (Sigma)+5 μl H2O2 per 10 ml pH 4.5 0.1M citrate buffer for 15 minutes in the dark at room temperature. The reaction was stopped with 50 μl HCl, and the optical density was read at 490 nm relative to 620 nm.

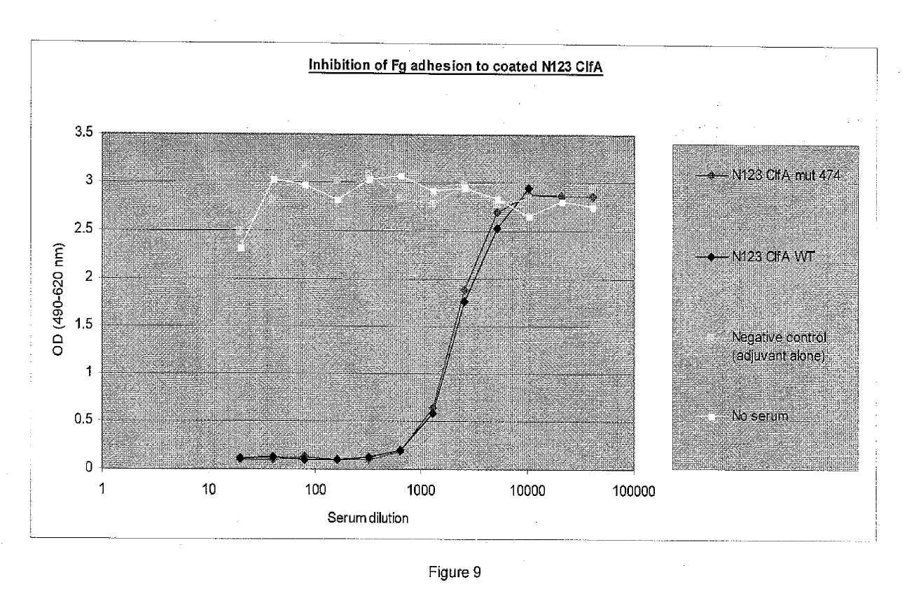

The results shown in FIG. 9 demonstrate that antibodies raised against both wild type and 474 mutant ClfA N123 were able to inhibit the binding of fibrinogen to ClfA N123 coated plates to about the same degree.

Inhibition Assay of S. aureus Adhesion to Coated Fibrinogen

Groups of 20 mice were inoculated intramuscularly with 10 μg of ClfA N123 or mutated 474 ClfA formulated with the adjuvant AS02V, on days 0, 14 and 28. A control group was inoculated with the adjuvant alone.

On day 42 serum was collected from the mice and pooled sera from each group were tested in an inhibition assay of S. aureus adhesion to coated fibrinogen.

Human fibrinogen (ref: SIGMA F4883-16) was coated at 10 μg/ml in phosphate buffered saline (PBS) on high binding microtitre plates (Nunc Maxisorp) overnight at 4° C. The plates were blocked with PBS-BSA 1% for 30 min at room temperature with shaking.

During this saturation step, serial two-fold dilutions (starting at 1/10) of the mice antisera were done in another microplate in PBS-BSA 0.2%-Tween 0.05%. Then, heat inactivated Newman D spa S. aureus bacteria (2 10e6 CFU/well) were added and the microplates were incubated at room temperature for 30 minutes with shaking.

After washing of the fibrinogen coated microplates, the mix antisera-bacteria was added and incubated at room temperature for 30 minutes with shaking.

After washing, the bound bacteria were detected using anti-killed whole cells rabbit polyclonal (obtained after immunization with killed S. aureus Lowenstein) diluted 1:50000 in PBS-BSA 0.2%-Tween 0.05% and incubated for 30 minutes at room temperature with shaking.

After washing, bound rabbit antibody was detected using Jackson ImmunoLaboratories Inc. peroxidase-conjugated affiniPure Goat Anti-Rabbit IgG (ref: 111-035-003) diluted 1:5000 in PBS-tween 0.05%. The detection antibodies were incubated for 30 minutes at room temperature with shaking.

The color was developed using 4 mg OPD (Sigma)+5 μl H2O2 per 10 ml pH 4.5 0.1M citrate buffer for 15 minutes in the dark at room temperature. The reaction was stopped with 50 μl HCl, and the optical density was read at 490 nm relative to 620 nm.

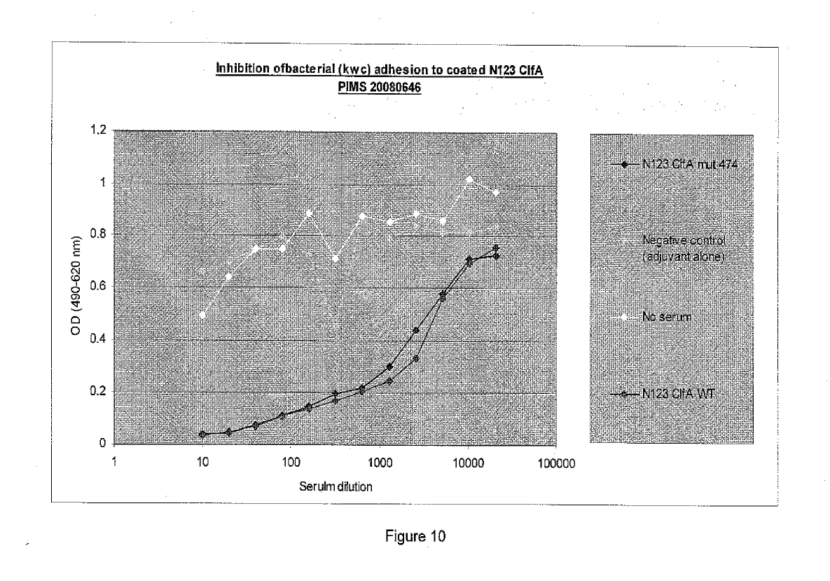

The results shown in FIG. 10 demonstrate that antibodies raised against both wild type and 474 mutant ClfA N123 were able to inhibit the binding of S. aureus bacteria to fibrinogen coated plates to about the same degree.

Sequences

SEQ ID NO: 1
MTKHYLNSKYQSEQRSSAMKKITMGTASIILGSLVYIGADSQQVNAATE
ATNATNNQSTQVSQATSQPINFQVQKDGSSEKSHMDDYMQHPGKVIKQN
NKYYFQTVLNNASFWKEYKFYNANNQELATTVVNDNKKADTRTINVAVE
PGYKSLTTKVHIVVPQINYNHRYTTHLEFEKAIPTLADAAKPNNVKPVQ
PKPAQPKTPTEQTKPVQPKVEKVKPTVTTTSKVEDNHSTKVVSTDTTKD
QTKTQTAHTVKTAQTAQEQNKVQTPVKDVATAKSESNNQAVSDNKSQQT
NKVTKHNETPKQASKAKELPKTGLTSVDNFISTVAFATLALLGSLSLLL
FKRKESK
SEQ ID NO: 2
STQVSQATSQPINFQVQKDGSSEKSHMDDYMQHPGKVIKQNNKYYFQTV
LNNASFWKEYKFYNANNQELATTVVNDNKKADTRTINVAVEPGYKSLTT
KVHIVVPQINYNHRYTTHLEFEKAIPTLADAAK
SEQ ID NO: 3
SQATSQPINFQVQKDGSSEKSHMDDYMQHPGKVIKQNNKYYFQTVLNNAS
FWKEYKFYNANNQELATTVVNDNKKADTRTINVAVEPGYKSLTTKVHIV
VPQINYNHRYTTHLEFEKAIPTLADA
SEQ ID NO: 4
DSQQVNAATEATNATNNQSTQVSQATSQPINFQVQKDGSSEKSHMDDYMQ
HPGKVIKQNNKYYFQTVLNNASFWKEYKFYNANNQELATTVVNDNKKADT
RTINVAVEPGYKSLTTKVHIVVPQINYNHRYTTHLEFEKAIPTLAD
AAK
SEQ ID NO: 5
SQATSQPINFQVQKDGSSEKSHMDDYMQHPGKVIKQNNKYYFQTVLNNA
SFWKEYKFYNANNQELATTVVNDNKKADTRTINVAVEPGYKSLTTKVHI
VVPQINYNHRYTTHLEFEKAIPTLA
SEQ ID NO: 6
MNKQQKEFKSFYSIRKSSLGVASVAISTLLLLMSNGEAQAAAEETGGTN
TEAQPKTEAVASPTTTSEKAPETKPVANAVSVSNKEVEAPTSETKEAKE
VKEVKAPKETKEVKPAAKATNNTYPILNQELREAIKNPAIKDKDHSAPN
SRPIDFEMKKKDGTQQFYHYASSVKPARVIFTDSKPEIELGLQSGQFWR
KFEVYEGDKKLPIKLVSYDTVKDYAYIRFSVSNGTKAVKIVSSTHFNNK
EEKYDYTLMEFAQPIYNSADKFKTEEDYKAEKLLAPYKKAKTLERQVYE
LNKIQDKLPEKLKAEYKKKLEDTKKALDEQVKSAITEFQNVQPTNEKMT
DLQDTKYVVYESVENNESMMDTFVKHPIKTGMLNGKKYMVMETTNDDYW
KDFMVEGQRVRTISKDAKNNTRTIIFPYVEGKTLYDAIVKVHVKTIDYD
GQYHVRIVDKEAFTKANTDKSNKKEQQDNSAKKEATPATPSKPTPSPVE
KESQKQDSQKDDNKQLPSVEKENDASSESGKDKTPATKPTKGEVESSSTT
PTKVVSTTQNVAKPTTASSKTTKDVVQTSAGSSEAKDSAPLQKANIKNT
NDGHTQSQNNKNTQENKAKSLPQTGEESNKDMTLPLMALLALSSIVAFV
LPRKRKN
SEQ ID NO: 7
DKDHSAPNSRPIDFEMKKKDGTQQFYHYASSVKPARVIFTDSKPEIELG
LQSGQFWRKFEVYEGDKKLPIKLVSYDTVKDYAYIRFSVSNGTKAVKIV
SSTHFNNKEEKYDYTLMEFAQPIYNSADKFKT
SEQ ID NO: 8
SAPNSRPIDFEMKKKDGTQQFYHYASSVKPARVIFTDSKPEIELGLQSGQ
FWRKFEVYEGDKKLPIKLVSYDTVKDYAYIRFSVSNGTKAVKIVSSTHF
NNKEEKYDYTLMEFAQPIYNSA
SEQ ID NO: 9
PTNEKMTDLQDTKYVVYESVENNESMMDTFVKHPIKTGMLNGKKYMVME
TTNDDYWKDFMVEGQRVRTISKDAKNNTRTIIFPYVEGKTLYDAIVKVH
VKTIDYDGQYHVRIVDKEAFTKANTDKS
SEQ ID NO: 10
KMTDLQDTKYVVYESVENNESMMDTFVKHPIKTGMLNGKKYMVMETTND
DYWKDFMVEGQRVRTISKDAKNNTRTIIFPYVEGKTLYDAIVKVHVKTI
DYDGQYHVRIVDKEAFTKANT
SEQ ID NO: 11
DKDHSAPNSRPIDFEMKKKDGTQQFYHYASSVKPARVIFTDSKPEIELG
LQSGQFWRKFEVYEGDKKLPIKLVSYDTVKDYAYIRFSVSNGTKAVKIV
SSTHFNNKEEKYDYTLMEFAQPIYNSADKFKTEEDYKAEKLLAPYKKAK
TLERQVYELNKIQDKLPEKLKAEYKKKLEDTKKALDEQVKSAITEFQNV
QPTNEKMTDLQDTKYVVYESVENNESMMDTFVKHPIKTGMLNGKKYMVM
ETTNDDYWKDFMVEGQRVRTISKDAKNNTRTIIFPYVEGKTLYDAIVKV
HVKTIDYDGQYHVRIVDKEAFTKANTDKS
SEQ ID NO: 12
SAPNSRPIDFEMKKKDGTQQFYHYASSVKPARVIFTDSKPEIELGLQSG
QFWRKFEVYEGDKKLPIKLVSYDTVKDYAYIRFSVSNGTKAVKIVSSTH
FNNKEEKYDYTLMEFAQPIYNSADKFKTEEDYKAEKLLAPYKKAKTLER
QVYELNKIQDKLPEKLKAEYKKKLEDTKKALDEQVKSAITEFQNVQPTN
EKMTDLQDTKYVVYESVENNESMMDTFVKHPIKTGMLNGKKYMVMETTN
DDYWKDFMVEGQRVRTISKDAKNNTRTIIFPYVEGKTLYDAIVKVHVKT
IDYDGQYHVRIVDKEAFTKANT
SEQ ID NO: 13
DKDHSAPNSRPIDFEMKKKDGTQQFYHYASSVKPARVIFTDSKPEIELG
LQSGQFWRKFEVYEGDKKLPIKLVSYDTVKDYAYIRFSVSNGTKAVKIV
SSTHFNNKEEKYDYTLMEFAQPIYNSADKFKTKLGGSPTNEKMTDLQDT
KYVVYESVENNESMMDTFVKHPIKTGMLNGKKYMVMETTNDDYWKDFMV
EGQRVRTISKDAKNNTRTIIFPYVEGKTLYDAIVKVHVKTIDYDGQYHV
RIVDKEAFTKANTDKS
SEQ ID NO: 14
MKNILKVFNTTILALIIIIATFSNSANAADSGTLNYEVYKYNTNDTSIA
NDYFNKPAKYIKKNGKLYVQITVNHSHWITGMSIEGHKENIISKNTAKD
ERTSEFEVSKLNGKIDGKIDVYIDEKVNGKPFKYDHHYNITYKFNGPTD
VAGANAPGKDDKNSASGSDKGSDGTTTGQSESNSSNKDKVENPQTNAGT
PAYIYAIPVASLALLIAITLFVRKKSKGNVE
SEQ ID NO: 15
NSANAADSGTLNYEVYKYNTNDTSIANDYFNKPAKYIKKNGKLYVQITV
NHSHWITGMSIEGHKENIISKNTAKDERTSEFEVSKLNGKIDGKIDVYI
DEKVNGKPFKYDHHYNITYKFNGPTDVAGANAP
SEQ ID NO: 16
MNKHHPKLRSFYSIRKSTLGVASVIVSTLFLITSQHQAQAAENTNTSDK
ISENQNNNATTTQPPKDTNQTQPATQPANTAKNYPAADESLKDAIKDPA
LENKEHDIGPREQVNFQLLDKNNETQYYHFFSIKDPADVYYTKKKAEVE
LDINTASTWKKFEVYENNQKLPVRLVSYSPVPEDHAYIRFPVSDGTQEL
KIVSSTQIDDGEETNYDYTKLVFAKPIYNDPSLVKSDTNDAVVTNDQSS
SVASNQTNTNTSNQNISTINNANNQPQATTNMSQPAQPKSSTNADQASS
QPAHETNSNGNTNDKTNESSNQSDVNQQYPPADESLQDAIKNPAIIDKE
HTADNWRPIDFQMKNDKGERQFYHYASTVEPATVIFTKTGPIIELGLKT
ASTWKKFEVYEGDKKLPVELVSYDSDKDYAYIRFPVSNGTREVKIVSSI
EYGENIHEDYDYTLMVFAQPITNNPDDYVDEETYNLQKLLAPYHKAKTL
ERQVYELEKLQEKLPEKYKAEYKKKLDQTRVELADQVKSAVTEFENVTP
TNDQLTDLQEAHFVVFESEENSESVMDGFVEHPFYTATLNGQKYVVMKT
KDDSYWKDLIVEGKRVTTVSKDPKNNSRTLIFPYIPDKAVYNAIVKVVV
ANIGYEGQYHVRIINQDINTKDDDTSQNNTSEPLNVQTGQEGKVADTDV
AENSSTATNPKDASDKADVIEPESDVVKDADNNIDKDVQHDVDHLSDMS
DNNHFDKYDLKEMDTQIAKDTDRNVDKDADNSVGMSSNVDTDKDSNKNK
DKVIQLNHIADKNNHTGKAAKLDVVKQNYNNTDKVTDKKTTEHLPSDIH
KTVDKTVKTKEKAGTPSKENKLSQSKMLPKTGETTSSQSWWGLYALLGM
LALFIPKFRKESK
SEQ ID NO: 17
NKEHDIGPREQVNFQLLDKNNETQYYHFFSIKDPADVYYTKKKAEVELD
INTASTWKKFEVYENNQKLPVRLVSYSPVPEDHAYIRFPVSDGTQELKI
VSSTQIDDGEETNYDYTKLVFAKPIYNDPSLVKS
SEQ ID NO: 18
KEHTADNWRPIDFQMKNDKGERQFYHYASTVEPATVIFTKTGPIIELGL
KTASTWKKFEVYEGDKKLPVELVSYDSDKDYAYIRFPVSNGTREVKIVS
SIEYGENIHEDYDYTLMVFAQPITNNPDDYVD
SEQ ID NO: 19
TNDQLTDLQEAHFVVFESEENSESVMDGFVEHPFYTATLNGQKYVVMKT
KDDSYWKDLIVEGKRVTTVSKDPKNNSRTLIFPYIPDKAVYNAIVKVV
VANIGYEGQYHVRIINQDINTKDDDTSQ
SEQ ID NO: 20
MNMKKKEKHAIRKKSIGVASVLVGTLIGFGLLSSKEADASENSVTQSDS
ASNESKSNDSSSVSAAPKTDDTNVSDTKTSSNTNNGETSVAQNPAQQET
TQSSSTNATTEETPVTGEATTTTTNQANTPATTQSSNTNAEELVNQTSN
ETTSNDTNTVSSVNSPQNSTNAENVSTTQDTSTEATPSNNESAPQSTDA
SNKDVVNQAVNTSAPRMRAFSLAAVAADAPVAGTDITNQLTNVTVGIDS
GTTVYPHQAGYVKLNYGFSVPNSAVKGDTFKITVPKELNLNGVTSTAKV
PPIMAGDQVLANGVIDSDGNVIYTFTDYVNTKDDVKATLTMPAYIDPEN
VKKTGNVTLATGIGSTTANKTVLVDYEKYGKFYNLSIKGTIDQIDKTNN
TYRQTIYVNPSGDNVIAPVLTGNLKPNTDSNALIDQQNTSIKVYKVDNA
ADLSESYFVNPENFEDVTNSVNITFPNPNQYKVEFNTPDDQITTPYIVV
VNGHIDPNSKGDLALRSTLYGYNSNIIWRSMSWDNEVAFNNGSGSGDGI
DKPVVPEQPDEPGEIEPIPEDSDSDPGSDSGSDSNSDSGSDSGSDSTSD
SGSDSASDSDSASDSDSASDSDSASDSDSASDSDSDNDSDSDSDSDSDS
DSDSDSDSDSDSDSDSDSDSDSDSDSDSDSDSDSDSDSDSDSDSDSDSD
SDSDSDSDSDSDSDSDSDSDSDSDSDSDSDSDSDSDSDSDSDSDSDSDS
DSDSDSDSDSDSDSDSDSDSDSDSDSDSDSDSASDSDSDSDSDSDSDSD
SDSDSDSDSDSDSDSDSDSDSDSESDSDSDSDSDSDSDSDSDSDSDSAS
DSDSGSDSDSSSDSDSESDSNSDSESVSNNNVVPPNSPKNGTNASNKNE
AKDSKEPLPDTGSEDEANTSLIWGLLASIGSLLLFRRKKENKDKK
SEQ ID NO: 21
MNMKKKEKHAIRKKSIGVASVLVGTLIGFGLLSSKEADASENSVTQSDS
ASNESKSNDSSSVSAAPKTDDTNVSDTKTSSNTNNGETSVAQNPAQQET
TQSSSTNATTEETPVTGEATTTTTNQANTPATTQSSNTNAEELVNQTSN
ETTSNDTNTVSSVNSPQNSTNAENVSTTQDTSTEATPSNNESAPQSTDA
SNKDVVNQAVNTSAPRMRAFSLAAVAADAPVAGTDITNQLTNVTVGIDS
GTTVYPHQAGYVKLNYGFSVPNSAVKGDTFKITVPKELNLNGVTSTAKV
PPIMAGDQVLANGVIDSDGNVIYTFTDYVNTKDDVKATLTMPAYIDPEN
VKKTGNVTLATGIGSTTANKTVLVDYEKYGKFYNLSIKGTIDQIDKTNN
TYRQTIYVNPSGDNVIAPVLTGNLKPNTDSNALIDQQNTSIKVYKVDNA
ADLSESYFVNPENFEDVTNSVNITFPNPNQYKVEFNTPDDQITTPYIVV
VNGHIDPNSKGDLALRSTLYGYNSNIIWRSMSWDNEVAFNNGSGSGDGID
KPVVPEQPDEPGEIEPIPE
SEQ ID NO: 22
SLAAVAADAPVAGTDITNQLTNVT
VGIDSGTTVYPHQAGYVKLNYGFSVPNSAVKGDTFKITVPKELNLNGVT
STAKVPPIMAGDQVLANGVIDSDGNVIYTFTDYVNTKDDVKATLTMPAY
IDPENVKKTGNVTLATGIGSTTANKTVLVDYEKYGKFYNLSIKGTIDQI
DKTNNTYRQTIYVNPSGDNVIAPVLTGNLKPNTDSNALIDQQNTSIKVY
KVDNAADLSESYFVNPENFEDVTNSVNITFPNPNQYKVEFNTPDDQITT
PYIVVVNGHIDPNSKGDLALRSTLYGYNSNIIWRSMSWDNEVAFNNGSG
SGDGIDKPVVPEQPDEPGEIEPIPE
SEQ ID NO: 23
GTDITNQLTNVTVGIDSGTTVYPHQAGYVKLNYGFSVPNSAVKGDTFKI
TVPKELNLNGVTSTAKVPPIMAGDQVLANGVIDSDGNVIYTFTDYVNTK
DDVKATLTMPAYIDPENVKKTGNVTLATGIGSTTANKTVLVDYEKYGKF
YNLSIKGTIDQIDKTNNTYRQTIYVNPSGDNVIAPVLTGNLKPNTDSNA
LIDQQNTSIKVYKVDNAADLSESYFVNPENFEDVTNSVNITFPNPNQYK
VEFNTPDDQITTPYIVVVNGHIDPNSKGDLALRSTLYGYNSNIIWRSMS
WDNEVAFNNGSGSGDGIDKPVVPEQPDEPGEIEPIPE
SEQ ID NO: 24
MAGTDITNQLTNVT
VGIDSGTTVYPHQAGYVKLNYGFSVPNSAVKGDTFKITVPKELNLNGVT
STAKVPPIMAGDQVLANGVIDSDGNVIYTFTDYVNTKDDVKATLTMSAA
IDPENVKKTGNVTLATGIGSTTANKTVLVDYEKYGKFYNLSIKGTIDQI
DKTNNTYRQTIYVNPSGDNVIAPVLTGNLKPNTDSNALIDQQNTSIKVY
KVDNAADLSESYFVNPENFEDVTNSVNITFPNPNQYKVEFNTPDDQITT
PYIVVVNGHIDPNSKGDLALRSTLYGYNSNIIWRSMSWDNEVAFNNGSG
SGDGIDKPVVPEQPDEPGEIEPIPE
SEQ ID NO: 25
LKKRIDYLSNKQNKYSIRRFTVGTTSVIVGATILFGIGNHQAQASEQSN
DTTQSSKNNASADSEKNNMIETPQLNTTANDTSDISANTNSANVDSTTK
PMSTQTSNTTTTEPASTNETPQPTAIKNQATAAKMQDQTVPQEANSQVD
NKTTNDANSIATNSELKNSQTLDLPQSSPQTISNAQGTSKPSVRTRAVR
SLAVAEPVVNAADAKGTNVNDKVTASNFKLEKTTFDPNQSGNTFMAANF
TVTDKVKSGDYFTAKLPDSLTGNGDVDYSNSNNTMPIADIKSTNGDVVA
KATYDILTKTYTFVFTDYVNNKENINGQFSLPLFTDRAKAPKSGTYDAN
INIADEMFNNKITYNYSSPIAGIDKPNGANISSQIIGVDTASGQNTYK
QTVFVNPKQRVLGNTWVYIKGYQDKIEESSGKVSATDTKLRIFEVNDTS
KLSDSYYADPNDSNLKEVTDQFKNRIYYEHPNVASIKFGDITKTYVVLV
EGHYDNTGKNLKTQVIQENVDPVTNRDYSIFGWNNENVVRYGGGSADGD
SAVNPKDPTPGPPVDPEPSPDPEPEPTPDPEPSPDPEPEPSPDPDPDSD
SDSDSGSDSDSGSDSDSESDSDSDSDSDSDSDSDSESDSDSESDSESDS
DSDSDSDSDSDSDSDSDSDSDSDSDSDSDSDSDSDSDSDSDSDSDSDSD
SDSDSDSDSDSDSDSDSDSDSDSDSDSDSDSDSDSDSDSDSDSDSDSDS
DSDSDSDSDSDSDSDSDSDSDSDSDSDSDSDSDSDSDSDSDSDSDSDSD
SDSDSDSDSDSDSDSDSDSDSRVTPPNNEQKAPSNPKGEVNHSNKVSKQ
HKTDALPETGDKSENTNATLFGAMMALLGSLLLFRKRKQDHKEKA
SEQ ID NO: 26
LKKRIDYLSNKQNKYSIRRFTVGTTSVIVGATILFGIGNHQAQASEQSN
DTTQSSKNNASADSEKNNMIETPQLNTTANDTSDISANTNSANVDSTTK
PMSTQTSNTTTTEPASTNETPQPTAIKNQATAAKMQDQTVPQEANSQVD
NKTTNDANSIATNSELKNSQTLDLPQSSPQTISNAQGTSKPSVRTRAVR
SLAVAEPVVNAADAKGTNVNDKVTASNFKLEKTTFDPNQSGNTFMAANF
TVTDKVKSGDYFTAKLPDSLTGNGDVDYSNSNNTMPIADIKSTNGDVVA
KATYDILTKTYTFVFTDYVNNKENINGQFSLPLFTDRAKAPKSGTYDAN
INIADEMFNNKITYNYSSPIAGIDKPNGANISSQIIGVDTASGQNTYKQ
TVFVNPKQRVLGNTWVYIKGYQDKIEESSGKVSATDTKLRIFEVNDTSK
LSDSYYADPNDSNLKEVTDQFKNRIYYEHPNVASIKFGDITKTYVVLVE
GHYDNTGKNLKTQVIQENVDPVTNRDYSIFGWNNENVVRYGGGSADGDSA
VN
SEQ ID NO: 27
SLAVAEPVVNAADAKGTNVNDKVTASNFKLEKTTFDPNQSGNTFMAANF
TVTDKVKSGDYFTAKLPDSLTGNGDVDYSNSNNTMPIADIKSTNGDVVA
KATYDILTKTYTFVFTDYVNNKENINGQFSLPLFTDRAKAPKSGTYDAN
INIADEMFNNKITYNYSSPIAGIDKPNGANISSQIIGVDTASGQNTYKQ
TVFVNPKQRVLGNTWVYIKGYQDKIEESSGKVSATDTKLRIFEVNDTSK
LSDSYYADPNDSNLKEVTDQFKNRIYYEHPNVASIKFGDITKTYVVLVE
GHYDNTGKNLKTQVIQENVDPVTNRDYSIFGWNNENVVRYGGGSADGDS
AVN
SEQ ID NO: 28
GTNVNDKVTASNFKLEKTTFDPNQSGNTF
MAANFTVTDKVKSGDYFTAKLPDSLTGNGDVDYSNSNNTMPIADIKSTN
GDVVAKATYDILTKTYTFVFTDYVNNKENINGQFSLPLFTDRAKAPKSG
TYDANINIADEMFNNKITYNYSSPIAGIDKPNGANISSQIIGVDTASGQ
NTYKQTVFVNPKQRVLGNTWVYIKGYQDKIEESSGKVSATDTKLRIFEV
NDTSKLSDSYYADPNDSNLKEVTDQFKNRIYYEHPNVASIKFGDITKTY
VVLVEGHYDNTGKNLKTQVIQENVDPVTNRDYSIFGWNNENVVRYGGGS
ADGDSAVN
SEQ ID NO: 29
MNNKKTATNRKGMIPNRLNKFSIRKYSVGTASILVGTTLIFGLSGHEAK
AAEHTNGELNQSKNETTAPSENKTTEKVDSRQLKDNTQTATADQPKVTM
SDSATVKETSSNMQSPQNATASQSTTQTSNVTTNDKSSTTYSNETDKSN
LTQAKNVSTTPKTTTIKQRALNRMAVNTVAAPQQGTNVNDKVHFTNIDI
AIDKGHVNKTTGNTEFWATSSDVLKLKANYTIDDSVKEGDTFTFKYGQY
FRPGSVRLPSQTQNLYNAQGNIIAKGIYDSKTNTTTYTFTNYVDQYTNV
SGSFEQVAFAKRENATTDKTAYKMEVTLGNDTYSKDVIVDYGNQKGQQL
ISSTNYINNEDLSRNMTVYVNQPKKTYTKETFVTNLTGYKFNPDAKNFK
IYEVTDQNQFVDSFTPDTSKLKDVTGQFDVIYSNDNKTATVDLLNGQSS
KSDKQYIIQQVAYPDNSSTDNGIDYTLETQNGKSSWSNSYSNVNGSSTA
NGDQKKYNLGDYVWEDTNKDGKQDANEKGIKGVYVILKDSNGKELDRTT
TDENGKYQFTGLSNGTYSVEFSTPAGYTPTTANAGTDDAVDSDGLTTTG
VIKDADNMTLDSGFYKTPKYSLGDYVWYDSNKDGKQDSTEKGIKGVKVT
LQNEKGEVIGTTETDENGKYRFDNLDSGKYKVIFEKPAGLTQTGTNTTE
DDKDADGGEVDVTITDHDDFTLDNGYYEEETSDSDSDSDSDSDSDSDSD
SDSDSDSDSDSDSDSDSDSDSDSDSDSDSDSDSDSDSESDSDSDSDSDS
DSDSDSDSDSDSDSDSDSDSDSDSDSDSDSDSDSDSDSDNDSDSDSDSD
SDSDSDSDSDSDSDSDSDSDSDSDSDSDSDSDSDSDSDSDSDSDSDSDS
DSDSDSDSDSDAGKHTPTKPMSTVKDQHKTAKALPETGSENNNSNNGTL
FGGLFAALGSLLLFGRRKKQNK
SEQ ID NO: 30
AEHTNGELNQSKNETTAPSENKTTEKVDSRQLKDNTQTATADQPKVTMS
DSATVKETSSNMQSPQNATASQSTTQTSNVTTNDKSSTTYSNETDKSNL
TQAKNVSTTPKTTTIKQRALNRMAVNTVAAPQQGTNVNDKVHFTNIDIA
IDKGHVNKTTGNTEFWATSSDVLKLKANYTIDDSVKEGDTFTFKYGQYF
RPGSVRLPSQTQNLYNAQGNIIAKGIYDSKTNTTTYTFTNYVDQYTNVS
GSFEQVAFAKRENATTDKTAYKMEVTLGNDTYSKDVIVDYGNQKGQQLI
SSTNYINNEDLSRNMTVYVNQPKKTYTKETFVTNLTGYKFNPDAKNFKI
YEVTDQNQFVDSFTPDTSKLKDVTGQFDVIYSNDN
SEQ ID NO: 31
VAAPQQGTNVNDKVHFTNIDIAIDKGHVNKTTGNTEFWATSSDVLKLKA
NYTIDDSVKEGDTFTFKYGQYFRPGSVRLPSQTQNLYNAQGNIIAKGIY
DSKTNTTTYTFTNYVDQYTNVSGSFEQVAFAKRENATTDKTAYKMEVTL
GNDTYSKDVIVDYGNQKGQQLISSTNYINNEDLSRNMTVYVNQPKKTYT
KETFVTNLTGYKFNPDAKNFKIYEVTDQNQFVDSFTPDTSKLKDVTGQF
DVIYSNDN
SEQ ID NO: 32
MNNKKTATNRKGMIPNRLNKFSIRKYSVGTASILVGTTLIFGLSGHEAK
AAEHTNGELNQSKNETTAPSENKTTKKVDSRQLKDNTQTATADQPKVTM
SDSATVKETSSNMQSPQNATANQSTTKTSNVTTNDKSSTTYSNETDKSN
LTQAKDVSTTPKTTTIKPRTLNRMAVNTVAAPQQGTNVNDKVHFSNIDI
AIDKGHVNQTTGKTEFWATSSDVLKLKANYTIDDSVKEGDTFTFKYGQY
FRPGSVRLPSQTQNLYNAQGNIIAKGIYDSTTNTTTYTFTNYVDQYTNV
RGSFEQVAFAKRKNATTDKTAYKMEVTLGNDTYSEEIIVDYGNKKAQPL
ISSTNYINNEDLSRNMTAYVNQPKNTYTKQTFVTNLTGYKFNPNAKNFK
IYEVTDQNQFVDSFTPDTSKLKDVTDQFDVIYSNDNKTATVDLMKGQTS
SNKQYIIQQVAYPDNSSTDNGKIDYTLDTDKTKYSWSNSYSNVNGSSTA
NGDQKKYNLGDYVWEDTNKDGKQDANEKGIKGVYVILKDSNGKELDRTT
TDENGKYQFTGLSNGTYSVEFSTPAGYTPTTANVGTDDAVDSDGLTTTG
VIKDADNMTLDSGFYKTPKYSLGDYVWYDSNKDGKQDSTEKGIKGVKVT
LQNEKGEVIGTTETDENGKYRFDNLDSGKYKVIFEKPAGLTQTGTNTTE
DDKDADGGEVDVTITDHDDFTLDNGYYEEETSDSDSDSDSDSDSDSDSD
SDSDSDSDSDSDSDSDSDSDSDSDSDSDSDSDSDSDSDSDSDSDSDSDS
DSDSDSDSDSDSDSDSDSDSDSDSDSDSDSDSDSDSDSDSDSDSDSDSD
SDSDSDSDSDSDSDSDSDSDSDSDNDSDSDSDSDSDSDSDSDSDSDSDS
DSDSDSDSDSDSDSDSDSDSDSDSDSDSDSDSDSDSDSDSDNDSDSDSD
SDSDAGKHTPAKPMSTVKDQHKTAKALPETGSENNNSNNGTLFGGLFAA
LGSLLLFGRRKKQNK
SEQ ID NO: 33
AEHTNGELNQSKNETTAPSENKTTKKVDSRQLKDNTQTATADQPKVTMS
DSATVKETSSNMQSPQNATANQSTTKTSNVTTNDKSSTTYSNETDKSNL
TQAKDVSTTPKTTTIKPRTLNRMAVNTVAAPQQGTNVNDKVHFSNIDIA
IDKGHVNQTTGKTEFWATSSDVLKLKANYTIDDSVKEGDTFTFKYGQYF
RPGSVRLPSQTQNLYNAQGNIIAKGIYDSTTNTTTYTFTNYVDQYTNVR
GSFEQVAFAKRKNATTDKTAYKMEVTLGNDTYSEEIIVDYGNKKAQPLI
SSTNYINNEDLSRNMTAYVNQPKNTYTKQTFVTNLTGYKFNPNAKNFKI
YEVTDQNQFVDSFTPDTSKLKDVTDQFDVIYSNDN
SEQ ID NO: 34
VAAPQQGTNVNDKVHFSNIDIAIDKGHVNQTTGKTEFWATSSDVLKLKA
NYTIDDSVKEGDTFTFKYGQYFRPGSVRLPSQTQNLYNAQGNIIAKGIY
DSTTNTTTYTFTNYVDQYTNVRGSFEQVAFAKRKNATTDKTAYKMEVTL
GNDTYSEEIIVDYGNKKAQPLISSTNYINNEDLSRNMTAYVNQPKNTYT
KQTFVTNLTGYKFNPNAKNFKIYEVTDQNQFVDSFTPDTSKLKDVTDQF
DVIYSNDN
SEQ ID NO: 35
MLNRENKTAITRKGMVSNRLNKFSIRKYTVGTASILVGTTLIFGLGNQE
AKAAESTNKELNEATTSASDNQSSDKVDMQQLNQEDNTKNDNQKEMVSS
QGNETTSNGNKLIEKESVQSTTGNKVEVSTAKSDEQASPKSTNEDLNTK
QTISNQEALQPDLQENKSVVNVQPTNEENKKVDAKTESTTLNVKSDAIK
SNDETLVDNNSNSNNENNADIILPKSTAPKRLNTRMRIAAVQPSSTEAK
NVNDLITSNTTLTVVDADKNNKIVPAQDYLSLKSQITVDDKVKSGDYFT
IKYSDTVQVYGLNPEDIKNIGDIKDPNNGETIATAKHDTANNLITYTFT
DYVDRFNSVQMGINYSIYMDADTIPVSKNDVEFNVTIGNTTTKTTANIQ
YPDYVVNEKNSIGSAFTETVSHVGNKENPGYYKQTIYVNPSENSLTNAK
LKVQAYHSSYPNNIGQINKDVTDIKIYQVPKGYTLNKGYDVNTKELTDV
TNQYLQKITYGDNNSAVIDFGNADSAYVVMVNTKFQYTNSESPTLVQMA
TLSSTGNKSVSTGNALGFTNNQSGGAGQEVYKIGNYVWEDTNKNGVQEL
GEKGVGNVTVTVFDNNTNTKVGEAVTKEDGSYLIPNLPNGDYRVEFSNL
PKGYEVTPSKQGNNEELDSNGLSSVITVNGKDNLSADLGIYKPKYNLGD
YVWEDTNKNGIQDQDEKGISGVTVTLKDENGNVLKTVTTDADGKYKFTD
LDNGNYKVEFTTPEGYTPTTVTSGSDIEKDSNGLTTTGVINGADNMTLD
SGFYKTPKYNLGNYVWEDTNKDGKQDSTEKGISGVTVTLKNENGEVLQT
TKTDKDGKYQFTGLENGTYKVEFETPSGYTPTQVGSGTDEGIDSNGTST
TGVIKDKDNDTIDSGFYKPTYNLGDYVWEDTNKNGVQDKDEKGISGVTV
TLKDENDKVLKTVTTDENGKYQFTDLNNGTYKVEFETPSGYTPTSVTSG
NDTEKDSNGLTTTGVIKDADNMTLDSGFYKTPKYSLGDYVWYDSNKDGK
QDSTEKGIKDVKVTLLNEKGEVIGTTKTDENGKYCFDNLDSGKYKVIFE
KPAGLTQTVTNTTEDDKDADGGEVDVTITDHDDFTLDNGYFEEDTSDSD
SDSDSDSDSDSDSDSDSDSDSDSDSDSDSDSDSDSDSDSDSDSDSDSDS
DSDSDSDSDSDSDSDSDSDSDSDSDSDSDSDSDSDSDSDSDSDSDSDSD
SDSDSDSDSDSDSDSDSDSDSDSDSDSDSDSDSDSDSDSDSDSDSDSDS
DSDSDSDSDSDSDSDAGKHTPVKPMSTTKDHHNKAKALPETGSENNGSN
NATLFGGLFAALGSLLLFGRRKKQNK
SEQ ID NO: 36
AESTNKELNEATTSASDNQSSDKVDMQQLNQEDNTKNDNQKEMVSSQGN
ETTSNGNKLIEKESVQSTTGNKVEVSTAKSDEQASPKSTNEDLNTKQTI
SNQEALQPDLQENKSVVNVQPTNEENKKVDAKTESTTLNVKSDAIKSND
ETLVDNNSNSNNENNADIILPKSTAPKRLNTRMRIAAVQPSSTEAKNVN
DLITSNTTLTVVDADKNNKIVPAQDYLSLKSQITVDDKVKSGDYFTIKY
SDTVQVYGLNPEDIKNIGDIKDPNNGETIATAKHDTANNLITYTFTDYV
DRFNSVQMGINYSIYMDADTIPVSKNDVEFNVTIGNTTTKTTANIQYPD
YVVNEKNSIGSAFTETVSHVGNKENPGYYKQTIYVNPSENSLTNAKLKV
QAYHSSYPNNIGQINKDVTDIKIYQVPKGYTLNKGYDVNTKELTDVTNQ
YLQKITYGDNNSAVIDFGNADSAYVVMVNTKFQYTNSESPTLVQMATLS
STGNKSVSTGNALGFTNNQSGGAGQE
SEQ ID NO: 37
IAAVQPSSTEAKNVNDLITSNTTLTVVDADKNNKIVPAQDYLSLKSQIT
VDDKVKSGDYFTIKYSDTVQVYGLNPEDIKNIGDIKDPNNGETIATAKH
DTANNLITYTFTDYVDRFNSVQMGINYSIYMDADTIPVSKNDVEFNVTI
GNTTTKTTANIQYPDYVVNEKNSIGSAFTETVSHVGNKENPGYYKQTIY
VNPSENSLTNAKLKVQAYHSSYPNNIGQINKDVTDIKIYQVPKGYTLNK
GYDVNTKELTDVTNQYLQKITYGDNNSAVIDFGNADSAYVVMVNTKFQY
TNSESPTLVQMATLSSTGNKSVSTGNALGFTNNQSGGAGQE
SEQ ID NO: 38
MINRDNKKAITKKGMISNRLNKFSIRKYTVGTASILVGTTLIFGLGNQE
AKAAENTSTENAKQDDATTSDNKEVVSETENNSTTENDSTNPIKKETNT
DSQPEAKEESTTSSTQQQQNNVTATTETKPQNIEKENVKPSTDKTATED
TSVILEEKKAPNYTNNDVTTKPSTSEIQTKPTTPQESTNIENSQPQPTP
SKVDNQVTDATNPKEPVNVSKEELKNNPEKLKELVRNDNNTDRSTKPVA
TAPTSVAPKRLNAKMRFAVAQPAAVASNNVNDLITVTKQTIKVGDGKDN
VAAAHDGKDIEYDTEFTIDNKVKKGDTMTINYDKNVIPSDLTDKNDPID
ITDPSGEVIAKGTFDKATKQITYTFTDYVDKYEDIKARLTLYSYIDKQA
VPNETSLNLTFATAGKETSQNVSVDYQDPMVHGDSNIQSIFTKLDENKQ
TIEQQIYVNPLKKTATNTKVDIAGSQVDDYGNIKLGNGSTIIDQNTEIK
VYKVNPNQQLPQSNRIYDFSQYEDVTSQFDNKKSFSNNVATLDFGDIN
SAYIIKVVSKYTPTSDGELDIAQGTSMRTTDKYGYYNYAGYSNFIVTSN
DTGGGDGTVKPEEKLYKIGDYVWEDVDKDGVQGTDSKEKPMANVLVTLT
YPDGTTKSVRTDANGHYEFGGLKDGETYTVKFETPAGYLPTKVNGTTDG
EKDSNGSSITVKINGKDDMSLDTGFYKEPKYNLGDYVWEDTNKDGIQDA
NEPGIKDVKVTLKDSTGKVIGTTTTDASGKYKFTDLDNGNYTVEFETPA
GYTPTVKNTTAEDKDSNGLTTTGVIKDADNMTLDSGFYKTPKYSLGDYV
WYDSNKDGKQDSTEKGIKDVKVTLLNEKGEVIGTTKTDENGKYRFDNLD
SGKYKVIFEKPAGLTQTVTNTTEDDKDADGGEVDVTITDHDDFILDNGY
FEEDTSDSDSDSDSDSDSDSDSDSDSDSDSDSDSDSDSDSDSDSDSDSD
SDSDSDSDSDSDSDSDSDSDSDSDSDSDSDSDSDSDSDSDSDSDSDSDS
DSDSDSDSDSDSDSDSDSDSDSDSDSDSDSDSDSDSDSDSDSDSDSDSD
SDSDAGKHTPVKPMSTTKDHHNKAKALPETGSENNGSNNATLFGGLFAA
LGSLLLFGRRKKQNK
SEQ ID NO: 39
AENTSTENAKQDDATTSDNKEVVSETENNSTTENDSTNPIKKETNTDSQ
PEAKEESTTSSTQQQQNNVTATTETKPQNIEKENVKPSTDKTATEDTSV
ILEEKKAPNYTNNDVTTKPSTSEIQTKPTTPQESTNIENSQPQPTPSKV
DNQVTDATNPKEPVNVSKEELKNNPEKLKELVRNDNNTDRSTKPVATAP
TSVAPKRLNAKMRFAVAQPAAVASNNVNDLITVTKQTIKVGDGKDNVAA
AHDGKDIEYDTEFTIDNKVKKGDTMTINYDKNVIPSDLTDKNDPIDITD
PSGEVIAKGTFDKATKQITYTFTDYVDKYEDIKARLTLYSYIDKQAVPN
ETSLNLTFATAGKETSQNVSVDYQDPMVHGDSNIQSIFTKLDENKQTIE
QQIYVNPLKKTATNTKVDIAGSQVDDYGNIKLGNGSTIIDQNTEIKVYK
VNPNQQLPQSNRIYDFSQYEDVTSQFDNKKSFSNNVATLDFGDINSAYI
IKVVSKYTPTSDGELDIAQGTSMRTTDKYGYYNYAGYSNFIVTSNDTGG
GDGTV
SEQ ID NO: 40
FAVAQPAAVASNNVNDLITVTKQTIKVGDGKDNVAAAHDGKDIEYDTEF
TIDNKVKKGDTMTINYDKNVIPSDLTDKNDPIDITDPSGEVIAKGTFDK
ATKQITYTFTDYVDKYEDIKARLTLYSYIDKQAVPNETSLNLTFATAGK
ETSQNVSVDYQDPMVHGDSNIQSIFTKLDENKQTIEQQIYVNPLKKTAT
NTKVDIAGSQVDDYGNIKLGNGSTIIDQNTEIKVYKVNPNQQLPQSNRI
YDFSQYEDVTSQFDNKKSFSNNVATLDFGDINSAYIIKVVSKYTPTSDG
ELDIAQGTSMRTTDKYGYYNYAGYSNFIVTSNDTGGGDGTV
SEQ ID NO: 41
MINRDNKKAITKKGMISNRLNKFSIRKYTVGTASILVGTTLIFGLGNQE
AKAAENTSTENAKQDDATTSDNKEVVSETENNSTTENDSTNPIKKETNT
DSQPEAKEESTTSSTQQQQNNVTATTETKPQNIEKENVKPSTDKTATED
TSVILEEKKAPNYTNNDVTTKPSTSEIQTKPTTPQESTNIENSQPQPTP
SKVDNQVTDATNPKEPVNVSKEELKNNPEKLKELVRNDNNTDRSTKPVA
TAPTSVAPKRLNAKMRFAVAQPAAVASNNVNDLITVTKQTIKVGDGKDN
VAAAHDGKDIEYDTEFTIDNKVKKGDTMTINYDKNVIPSDLTDKNDPID
ITDPSGEVIAKGTFDKATKQITYTFTDYVDKYEDIKARLTLYSYIDKQA
VPNETSLNLTFATAGKETSQNVSVDYQDPMVHGDSNIQSIFTKLDENKQ
TIEQQIYVNPLKKTATNTKVDIAGSQVDDYGNIKLGNGSTIIDQNTEIK
VYKVNPNQQLPQSNRIYDFSQYEDVTSQFDNKKSFSNNVATLDFGDINS
AYIIKVVSKYTPTSDGELDIAQGTSMRTTDKYGYYNYAGYSNFIVTSND
TGGGDGTVKPEEKLYKIGDYVWEDVDKDGVQGTDSKEKPMANVLVTLTY
PDGTTKSVRTDANGHYEFGGLKDGETYTVKFETPAGYLPTKVNGTTDGE
KDSNGSSITVKINGKDDMSLDTGFYKEPKYNLGDYVWEDTNKDGIQDAN
EPGIKDVKVTLKDSTGKVIGTTTTDASGKYKFTDLDNGNYTVEFETPAG
YTPTVKNTTAEDKDSNGLTTTGVIKDADNMTLDSGFYKTPKYSLGDYVW
YDSNKDGKQDSTEKGIKDVKVTLLNEKGEVIGTTKTDENGKYRFDNLDS
GKYKVIFEKPAGLTQTVTNTTEDDKDADGGEVDVTITDHDDFILDNGYF
EEDTSDSDSDSDSDSDSDSDSDSDSDSDSDSDSDSDSDSDSDSDSDSDS
DSDSDSDSDSDSDSDSDSDSDSDSDSDSDSDSDSDSDSDSDSDSDSDSD
SDSDSDSDSDSDSDSDSDSDSDSDSDSDSDSDSDSDSDSDSDSDSDSDS
DSDAGKHTPVKPMSTTKDHHNKAKALPETGSENNGSNNATLFGGLFAAL
GSLLLFGRRKKQNK
SEQ ID NO: 42
AENTSTENAKQDDATTSDNKEVVSETENNSTTENDSTNPIKKETNTDSQ
PEAKEESTTSSTQQQQNNVTATTETKPQNIEKENVKPSTDKTATEDTSV
ILEEKKAPNYTNNDVTTKPSTSEIQTKPTTPQESTNIENSQPQPTPSKV
DNQVTDATNPKEPVNVSKEELKNNPEKLKELVRNDNNTDRSTKPVATAP
TSVAPKRLNAKMRFAVAQPAAVASNNVNDLITVTKQTIKVGDGKDNVAA
AHDGKDIEYDTEFTIDNKVKKGDTMTINYDKNVIPSDLTDKNDPIDITD
PSGEVIAKGTFDKATKQITYTFTDYVDKYEDIKARLTLYSYIDKQAVPN
ETSLNLTFATAGKETSQNVSVDYQDPMVHGDSNIQSIFTKLDENKQTIE
QQIYVNPLKKTATNTKVDIAGSQVDDYGNIKLGNGSTIIDQNTEIKVYK
VNPNQQLPQSNRIYDFSQYEDVTSQFDNKKSFSNN
SEQ ID NO: 43
FAVAQPAAVASNNVNDLITVTKQTIKVGDGKDNVAAAHDGKDIEYDTEF
TIDNKVKKGDTMTINYDKNVIPSDLTDKNDPIDITDPSGEVIAKGTFDK
ATKQITYTFTDYVDKYEDIKARLTLYSYIDKQAVPNETSLNLTFATAGK
ETSQNVSVDYQDPMVHGDSNIQSIFTKLDENKQTIEQQIYVNPLKKTAT
NTKVDIAGSQVDDYGNIKLGNGSTIIDQNTEIKVYKVNPNQQLPQSNRI
YDFSQYEDVTSQFDNKKSFSNN
SEQ ID NO: 44
MINKKNNLLTKKKPIANKSNKYAIRKFTVGTASIVIGATLLFGLGHNEA
KAEENSVQDVKDSNTDDELSDSNDQSSDEEKNDVINNNQSINTDDNNQI
IKKEETNNYDGIEKRSEDRTESTTNVDENEATFLQKTPQDNTHLTEEEV
KESSSVESSNSSIDTAQQPSHTTINREESVQTSDNVEDSHVSDFANSKI
KESNTESGKEENTIEQPNKVKEDSTTSQPSGYTNIDEKISNQDELLNLP
INEYENKARPLSTTSAQPSIKRVTVNQLAAEQGSNVNHLIKVTDQSITE
GYDDSEGVIKAHDAENLIYDVTFEVDDKVKSGDTMTVDIDKNTVPSDLT
DSFTIPKIKDNSGEIIATGTYDNKNKQITYTFTDYVDKYENIKAHLKLT
SYIDKSKVPNNNTKLDVEYKTALSSVNKTITVEYQRPNENRTANLQSMF
TNIDTKNHTVEQTIYINPLRYSAKETNVNISGNGDEGSTIIDDSTIIKV
YKVGDNQNLPDSNRIYDYSEYEDVTNDDYAQLGNNNDVNINFGNIDSPY
IIKVISKYDPNKDDYTTIQQTVTMQTTINEYTGEFRTASYDNTIAFSTS
SGQGQGDLPPEKTYKIGDYVWEDVDKDGIQNTNDNEKPLSNVLVTLTYP
DGTSKSVRTDEDGKYQFDGLKNGLTYKITFETPEGYTPTLKHSGTNPAL
DSEGNSVWVTINGQDDMTIDSGFYQTPKYSLGNYVWYDTNKDGIQGDDE
KGISGVKVTLKDENGNIISTTTTDENGKYQFDNLNSGNYIVHFDKPSGM
TQTTTDSGDDDEQDADGEEVHVTITDHDDFSIDNGYYDDESDSDSDSDS
DSDSDSDSDSDSDSDSDSDSDSDSDSDSDSDSDSDSDSDSDSDSDSDSD
SDSDSDSDSDSDSDSDSDSDSDSDSDSDSDSDSDSDSDSDSDSDSDSDS
DSDSDSDSDSDSDSDSDSDSDSDSDSDSDSDSDSDSDSDSDSDSDSDSD
SDSDSDSDSDSDSDSDSDSDNDSDLGNSSDKSTKDKLPDTGANEDYGSK
GTLLGTLFAGLGALLLGKRRKNRKNKN
SEQ ID NO: 45
EENSVQDVKDSNTDDELSDSNDQSSDEEKNDVINNNQSINTDDNNQIIK
KEETNNYDGIEKRSEDRTESTTNVDENEATFLQKTPQDNTHLTEEEVKE
SSSVESSNSSIDTAQQPSHTTINREESVQTSDNVEDSHVSDFANSKIKE
SNTESGKEENTIEQPNKVKEDSTTSQPSGYTNIDEKISNQDELLNLPIN
EYENKARPLSTTSAQPSIKRVTVNQLAAEQGSNVNHLIKVTDQSITEGY
DDSEGVIKAHDAENLIYDVTFEVDDKVKSGDTMTVDIDKNTVPSDLTDS
FTIPKIKDNSGEIIATGTYDNKNKQITYTFTDYVDKYENIKAHLKLTSY
IDKSKVPNNNTKLDVEYKTALSSVNKTITVEYQRPNENRTANLQSMFTN
IDTKNHTVEQTTYINPLRYSAKETNVNISGNGDEGSTIIDDSTIIKVYK
VGDNQNLPDSNRIYDYSEYEDVTNDDYAQLGNNN
SEQ ID NO: 46
VTVNQLAAEQGSNVNHLIKVTDQSITEGYDDSEGVIKAHDAENLIYDVT
FEVDDKVKSGDTMTVDIDKNTVPSDLTDSFTIPKIKDNSGEIIATGTYD
NKNKQITYTFTDYVDKYENIKAHLKLTSYIDKSKVPNNNTKLDVEYKTA
LSSVNKTITVEYQRPNENRTANLQSMFTNIDTKNHTVEQTIYINPLRYS
AKETNVNISGNGDEGSTIIDDSTIIKVYKVGDNQNLPDSNRIYDYSEYE
DVTNDDYAQLGNNN
In the following sequences, x denotes either a
covalent bond or 1-200 amino acids.
SEQ ID NO: 47
STQVSQATSQPINFQVQKDGSSEKSHMDDYMQHPGKVIKQNNKYYFQTV
LNNASFWKEYKFYNANNQELATTVVNDNKKADTRTINVAVEPGYKSLTT
KVHIVVPQINYNHRYTTHLEFEKAIPTLADAAKxSLAAVAADAPVAGTD
ITNQLTNVTVGIDSGTTVYPHQAGYVKLNYGFSVPNSAVKGDTFKITVP
KELNLNGVTSTAKVPPIMAGDQVLANGVIDSDGNVIYTFTDYVNTKDDV
KATLTMPAYIDPENVKKTGNVTLATGIGSTTANKTVLVDYEKYGKFYNL
SIKGTIDQIDKTNNTYRQTIYVNPSGDNVIAPVLTGNLKPNTDSNALID
QQNTSIKVYKVDNAADLSESYFVNPENFEDVTNSVNITFPNPNQYKVEF
NTPDDQITTPYIVVVNGHIDPNSKGDLALRSTLYGYNSNIIWRSMSWDN
EVAFNNGSGSGDGIDKPVVPEQPDEPGEIEPIPE
SEQ ID NO: 48
STQVSQATSQPINFQVQKDGSSEKSHMDDYMQHPGKVIKQNNKYYFQTV
LNNASFWKEYKFYNANNQELATTVVNDNKKADTRTINVAVEPGYKSLTT
KVHIVVPQINYNHRYTTHLEFEKAIPTLADAAKxSLAVAEPVVNAADAK
GTNVNDKVTASNFKLEKTTFDPNQSGNTFMAANFTVTDKVKSGDYFTAK
LPDSLTGNGDVDYSNSNNTMPIADIKSTNGDVVAKATYDILTKTYTFVF
TDYVNNKENINGQFSLPLFTDRAKAPKSGTYDANINIADEMFNNKITYN
YSSPIAGIDKPNGANISSQIIGVDTASGQNTYKQTVFVNPKQRVLGNTW
VYIKGYQDKIEESSGKVSATDTKLRIFEVNDTSKLSDSYYADPNDSNLK
EVTDQFKNRIYYEHPNVASIKFGDITKTYVVLVEGHYDNTGKNLKTQVI
QENVDPVTNRDYSIFGWNNENVVRYGGGSADGDSAVN
SEQ ID NO: 49
STQVSQATSQPINFQVQKDGSSEKSHMDDYMQHPGKVIKQNNKYYFQTV
LNNASFWKEYKFYNANNQELATTVVNDNKKADTRTINVAVEPGYKSLTT
KVHIVVPQINYNHRYTTHLEFEKAIPTLADAAKxVAAPQQGTNVNDKVH
FSNIDIAIDKGHVNQTTGKTEFWATSSDVLKLKANYTIDDSVKEGDTFT
FKYGQYFRPGSVRLPSQTQNLYNAQGNIIAKGIYDSTTNTTTYTFTNYV
DQYTNVRGSFEQVAFAKRKNATTDKTAYKMEVTLGNDTYSEEIIVDYGN
KKAQPLISSTNYINNEDLSRNMTAYVNQPKNTYTKQTFVTNLTGYKFNP
NAKNFKIYEVTDQNQFVDSFTPDTSKLKDVTDQFDVIYSNDN
SEQ ID NO: 50
STQVSQATSQPINFQVQKDGSSEKSHMDDYMQHPGKVIKQNNKYYFQTV
LNNASFWKEYKFYNANNQELATTVVNDNKKADTRTINVAVEPGYKSLTT
KVHIVVPQINYNHRYTTHLEFEKAIPTLADAAKxIAAVQPSSTEAKNVN
DLITSNTTLTVVDADKNNKIVPAQDYLSLKSQITVDDKVKSGDYFTIKY
SDTVQVYGLNPEDIKNIGDIKDPNNGETIATAKHDTANNLITYTFTDYV
DRFNSVQMGINYSIYMDADTIPVSKNDVEFNVTIGNTTTKTTANIQYPD
YVVNEKNSIGSAFTETVSHVGNKENPGYYKQTIYVNPSENSLTNAKLKV
QAYHSSYPNNIGQINKDVTDIKIYQVPKGYTLNKGYDVNTKELTDVTNQ
YLQKITYGDNNSAVIDFGNADSAYVVMVNTKFQYTNSESPTLVQMATLS
STGNKSVSTGNALGFTNNQSGGAGQE
SEQ ID NO: 51
STQVSQATSQPINFQVQKDGSSEKSHMDDYMQHPGKVIKQNNKYYFQTV
LNNASFWKEYKFYNANNQELATTVVNDNKKADTRTINVAVEPGYKSLTT
KVHIVVPQINYNHRYTTHLEFEKAIPTLADAAKxFAVAQPAAVASNNVN
DLITVTKQTIKVGDGKDNVAAAHDGKDIEYDTEFTIDNKVKKGDTMTIN
YDKNVIPSDLTDKNDPIDITDPSGEVIAKGTFDKATKQITYTFTDYVDK
YEDIKARLTLYSYIDKQAVPNETSLNLTFATAGKETSQNVSVDYQDPMV
HGDSNIQSIFTKLDENKQTIEQQIYVNPLKKTATNTKVDIAGSQVDDYG
NIKLGNGSTIIDQNTEIKVYKVNPNQQLPQSNRIYDFSQYEDVTSQFDN
KKSFSNNVATLDFGDINSAYIIKVVSKYTPTSDGELDIAQGTSMRTTDK
YGYYNYAGYSNFIVTSNDTGGGDGTV
SEQ ID NO: 52
STQVSQATSQPINFQVQKDGSSEKSHMDDYMQHPGKVIKQNNKYYFQTV
LNNASFWKEYKFYNANNQELATTVVNDNKKADTRTINVAVEPGYKSLTT
KVHIVVPQINYNHRYTTHLEFEKAIPTLADAAKxVTVNQLAAEQGSNVN
HLIKVTDQSITEGYDDSEGVIKAHDAENLIYDVTFEVDDKVKSGDTMTV
DIDKNTVPSDLTDSFTIPKIKDNSGEIIATGTYDNKNKQITYTFTDYVD
KYENIKAHLKLTSYIDKSKVPNNNTKLDVEYKTALSSVNKTITVEYQRP
NENRTANLQSMFTNIDTKNHTVEQTIYINPLRYSAKETNVNISGNGDEG
STIIDDSTIIKVYKVGDNQNLPDSNRIYDYSEYEDVTNDDYAQLGNNN
SEQ ID NO: 53
SLAAVAADAPVAGTDITNQLTNVT
VGIDSGTTVYPHQAGYVKLNYGFSVPNSAVKGDTFKITVPKELNLNGVT
STAKVPPIMAGDQVLANGVIDSDGNVIYTFTDYVNTKDDVKATLTMPAY
IDPENVKKTGNVTLATGIGSTTANKTVLVDYEKYGKFYNLSIKGTIDQI
DKTNNTYRQTIYVNPSGDNVIAPVLTGNLKPNTDSNALIDQQNTSIKVY
KVDNAADLSESYFVNPENFEDVTNSVNITFPNPNQYKVEFNTPDDQITT
PYIVVVNGHIDPNSKGDLALRSTLYGYNSNIIWRSMSWDNEVAFNNGSG
SGDGIDKPVVPEQPDEPGEIEPIPE
xSTQVSQATSQPINFQVQKDGSSEKSHMDDYMQHPGKVIKQNNKYYFQT
VLNNASFWKEYKFYNANNQELATTVVNDNKKADTRTINVAVEPGYKSLT
TKVHIVVPQINYNHRYTTHLEFEKAIPTLADAAK
SEQ ID NO: 54
SLAVAEPVVNAADAKGTNVNDKVTASNFKLEKTTFDPNQSGNTFMAANF
TVTDKVKSGDYFTAKLPDSLTGNGDVDYSNSNNTMPIADIKSTNGDVVA
KATYDILTKTYTFVFTDYVNNKENINGQFSLPLFTDRAKAPKSGTYDAN
INIADEMFNNKITYNYSSPIAGIDKPNGANISSQIIGVDTASGQNTYKQ
TVFVNPKQRVLGNTWVYIKGYQDKIEESSGKVSATDTKLRIFEVNDTSK
LSDSYYADPNDSNLKEVTDQFKNRIYYEHPNVASIKFGDITKTYVVLVE
GHYDNTGKNLKTQVIQENVDPVTNRDYSIFGWNNENVVRYGGGSADGDS
AVNxSTQVSQATSQPINFQVQKDGSSEKSHMDDYMQHPGKVIKQNNKYY
FQTVLNNASFWKEYKFYNANNQELATTVVNDNKKADTRTINVAVEPGYK
SLTTKVHIVVPQINYNHRYTTHLEFEKAIPTLADAAK
SEQ ID NO: 55
VAAPQQGTNVNDKVHFSNIDIAIDKGHVNQTTGKTEFWATSSDVLKLKA
NYTIDDSVKEGDTFTFKYGQYFRPGSVRLPSQTQNLYNAQGNIIAKGIY
DSTTNTTTYTFTNYVDQYTNVRGSFEQVAFAKRKNATTDKTAYKMEVTL
GNDTYSEEIIVDYGNKKAQPLISSTNYINNEDLSRNMTAYVNQPKNTYT
KQTFVTNLTGYKFNPNAKNFKIYEVTDQNQFVDSFTPDTSKLKDVTDQF
DVIYSNDNxSTQVSQATSQPINFQVQKDGSSEKSHMDDYMQHPGKVIKQ
NNKYYFQTVLNNASFWKEYKFYNANNQELATTVVNDNKKADTRTINVAV
EPGYKSLTTKVHIVVPQINYNHRYTTHLEFEKAIPTLADAAK
SEQ ID NO: 56
IAAVQPSSTEAKNVNDLITSNTTLTVVDADKNNKIVPAQDYLSLKSQIT
VDDKVKSGDYFTIKYSDTVQVYGLNPEDIKNIGDIKDPNNGETIATAKH
DTANNLITYTFTDYVDRFNSVQMGINYSIYMDADTIPVSKNDVEFNVTI
GNTTTKTTANIQYPDYVVNEKNSIGSAFTETVSHVGNKENPGYYKQTIY
VNPSENSLTNAKLKVQAYHSSYPNNIGQINKDVTDIKIYQVPKGYTLNK
GYDVNTKELTDVTNQYLQKITYGDNNSAVIDFGNADSAYVVMVNTKFQY
TNSESPTLVQMATLSSTGNKSVSTGNALGFTNNQSGGAGQExSTQVSQA
TSQPINFQVQKDGSSEKSHMDDYMQHPGKVIKQNNKYYFQTVLNNASFW
KEYKFYNANNQELATTVVNDNKKADTRTINVAVEPGYKSLTTKVHIVVP
QINYNHRYTTHLEFEKAIPTLADAAK
SEQ ID NO: 57
FAVAQPAAVASNNVNDLITVTKQTIKVGDGKDNVAAAHDGKDIEYDTEF
TIDNKVKKGDTMTINYDKNVIPSDLTDKNDPIDITDPSGEVIAKGTFDK
ATKQITYTFTDYVDKYEDIKARLTLYSYIDKQAVPNETSLNLTFATAGK
ETSQNVSVDYQDPMVHGDSNIQSIFTKLDENKQTIEQQIYVNPLKKTAT
NTKVDIAGSQVDDYGNIKLGNGSTIIDQNTEIKVYKVNPNQQLPQSNRI
YDFSQYEDVTSQFDNKKSFSNNVATLDFGDINSAYIIKVVSKYTPTSDG
ELDIAQGTSMRTTDKYGYYNYAGYSNFIVTSNDTGGGDGTVxSTQVSQA
TSQPINFQVQKDGSSEKSHMDDYMQHPGKVIKQNNKYYFQTVLNNASFW
KEYKFYNANNQELATTVVNDNKKADTRTINVAVEPGYKSLTTKVHIVVP
QINYNHRYTTHLEFEKAIPTLADAAK
SEQ ID NO: 58
VTVNQLAAEQGSNVNHLIKVTDQSITEGYDDSEGVIKAHDAENLIYDVT
FEVDDKVKSGDTMTVDIDKNTVPSDLTDSFTIPKIKDNSGEIIATGTYD
NKNKQITYTFTDYVDKYENIKAHLKLTSYIDKSKVPNNNTKLDVEYKTA
LSSVNKTITVEYQRPNENRTANLQSMFTNIDTKNHTVEQTIYINPLRYS
AKETNVNISGNGDEGSTIIDDSTIIKVYKVGDNQNLPDSNRIYDYSEYE
DVTNDDYAQLGNNNxSTQVSQATSQPINFQVQKDGSSEKSHMDDYMQHP
GKVIKQNNKYYFQTVLNNASFWKEYKFYNANNQELATTVVNDNKKADTR
TINVAVEPGYKSLTTKVHIVVPQINYNHRYTTHLEFEKAIPTLADAAK
SEQ ID NO: 59
DKDHSAPNSRPIDFEMKKKDGTQQFYHYASSVKPARVIFTDSKPEIELG
LQSGQFWRKFEVYEGDKKLPIKLVSYDTVKDYAYIRFSVSNGTKAVKIV
SSTHENNKEEKYDYTLMEFAQPIYNSADKFKTxSLAAVAADAPVAGTDI
TNQLTNVTVGIDSGTTVYPHQAGYVKLNYGFSVPNSAVKGDTFKITVPK
ELNLNGVTSTAKVPPIMAGDQVLANGVIDSDGNVIYTFTDYVNTKDDVK
ATLTMPAYIDPENVKKTGNVTLATGIGSTTANKTVLVDYEKYGKFYNLS
IKGTIDQIDKTNNTYRQTIYVNPSGDNVIAPVLTGNLKPNTDSNALIDQ
QNTSIKVYKVDNAADLSESYFVNPENFEDVTNSVNITFPNPNQYKVEFN
TPDDQITTPYIVVVNGHIDPNSKGDLALRSTLYGYNSNIIWRSMSWDNE
VAFNNGSGSGDGIDKPVVPEQPDEPGEIEPIPE
SEQ ID NO: 60
DKDHSAPNSRPIDFEMKKKDGTQQFYHYASSVKPARVIFTDSKPEIELG
LQSGQFWRKFEVYEGDKKLPIKLVSYDTVKDYAYIRFSVSNGTKAVKIV
SSTHFNNKEEKYDYTLMEFAQPIYNSADKFKTxSLAVAEPVVNAADAKG
TNVNDKVTASNFKLEKTTFDPNQSGNTFMAANFTVTDKVKSGDYFTAKL
PDSLTGNGDVDYSNSNNTMPIADIKSTNGDVVAKATYDILTKTYTFVFT
DYVNNKENINGQFSLPLFTDRAKAPKSGTYDANINIADEMFNNKITYNY
SSPIAGIDKPNGANISSQIIGVDTASGQNTYKQTVFVNPKQRVLGNTWV
YIKGYQDKIEESSGKVSATDTKLRIFEVNDTSKLSDSYYADPNDSNLKE
VTDQFKNRIYYEHPNVASIKFGDITKTYVVLVEGHYDNTGKNLKTQVIQ
ENVDPVTNRDYSIFGWNNENVVRYGGGSADGDSAVN
SEQ ID NO: 61
DKDHSAPNSRPIDFEMKKKDGTQQFYHYASSVKPARVIFTDSKPEIELG
LQSGQFWRKFEVYEGDKKLPIKLVSYDTVKDYAYIRFSVSNGTKAVKIV
SSTHFNNKEEKYDYTLMEFAQPIYNSADKFKTxVAAPQQGTNVNDKVHF
SNIDIAIDKGHVNQTTGKTEFWATSSDVLKLKANYTIDDSVKEGDTFTF
KYGQYFRPGSVRLPSQTQNLYNAQGNIIAKGIYDSTTNTTTYTFTNYVD
QYTNVRGSFEQVAFAKRKNATTDKTAYKMEVTLGNDTYSEEIIVDYGNK
KAQPLISSTNYINNEDLSRNMTAYVNQPKNTYTKQTFVTNLTGYKFNPN
AKNFKIYEVTDQNQFVDSFTPDTSKLKDVTDQFDVIYSNDN
SEQ ID NO: 62
DKDHSAPNSRPIDFEMKKKDGTQQFYHYASSVKPARVIFTDSKPEIELG
LQSGQFWRKFEVYEGDKKLPIKLVSYDTVKDYAYIRFSVSNGTKAVKIV
SSTHFNNKEEKYDYTLMEFAQPIYNSADKFKTxIAAVQPSSTEAKNVND
LITSNTTLTVVDADKNNKIVPAQDYLSLKSQITVDDKVKSGDYFTIKYS
DTVQVYGLNPEDIKNIGDIKDPNNGETIATAKHDTANNLITYTFTDYVD
RENSVQMGINYSIYMDADTIPVSKNDVEFNVTIGNTTTKTTANIQYPDY
VVNEKNSIGSAFTETVSHVGNKENPGYYKQTIYVNPSENSLTNAKLKVQ
AYHSSYPNNIGQINKDVTDIKIYQVPKGYTLNKGYDVNTKELTDVTNQY
LQKITYGDNNSAVIDFGNADSAYVVMVNTKFQYTNSESPTLVQMATLSS
TGNKSVSTGNALGFTNNQSGGAGQE
SEQ ID NO: 63
DKDHSAPNSRPIDFEMKKKDGTQQFYHYASSVKPARVIFTDSKPEIELG
LQSGQFWRKFEVYEGDKKLPIKLVSYDTVKDYAYIRFSVSNGTKAVKIV
SSTHFNNKEEKYDYTLMEFAQPIYNSADKFKTxFAVAQPAAVASNNVND
LITVTKQTIKVGDGKDNVAAAHDGKDIEYDTEFTIDNKVKKGDTMTINY
DKNVIPSDLTDKNDPIDITDPSGEVIAKGTFDKATKQITYTFTDYVDKY
EDIKARLTLYSYIDKQAVPNETSLNLTFATAGKETSQNVSVDYQDPMVH
GDSNIQSIFTKLDENKQTIEQQIYVNPLKKTATNTKVDIAGSQVDDYGN
IKLGNGSTIIDQNTEIKVYKVNPNQQLPQSNRIYDFSQYEDVTSQFDNK
KSFSNNVATLDFGDINSAYIIKVVSKYTPTSDGELDIAQGTSMRTTDKY
GYYNYAGYSNFIVTSNDTGGGDGTV
SEQ ID NO: 64
DKDHSAPNSRPIDFEMKKKDGTQQFYHYASSVKPARVIFTDSKPEIELG
LQSGQFWRKFEVYEGDKKLPIKLVSYDTVKDYAYIRFSVSNGTKAVKIV
SSTHFNNKEEKYDYTLMEFAQPIYNSADKFKTxVTVNQLAAEQGSNVNH
LIKVTDQSITEGYDDSEGVIKAHDAENLIYDVTFEVDDKVKSGDTMTVD
IDKNTVPSDLTDSFTIPKIKDNSGEIIATGTYDNKNKQITYTFTDYVDK
YENIKAHLKLTSYIDKSKVPNNNTKLDVEYKTALSSVNKTITVEYQRPN
ENRTANLQSMFTNIDTKNHTVEQTIYINPLRYSAKETNVNISGNGDEGS
TIIDDSTIIKVYKVGDNQNLPDSNRIYDYSEYEDVTNDDYAQLGNNN
SEQ ID NO: 65
SLAAVAADAPVAGTDITNQLTNVT
VGIDSGTTVYPHQAGYVKLNYGFSVPNSAVKGDTFKITVPKELNLNGVT
STAKVPPIMAGDQVLANGVIDSDGNVIYTFTDYVNTKDDVKATLTMPAY
IDPENVKKTGNVTLATGIGSTTANKTVLVDYEKYGKFYNLSIKGTIDQI
DKTNNTYRQTIYVNPSGDNVIAPVLTGNLKPNTDSNALIDQQNTSIKVY
KVDNAADLSESYFVNPENFEDVTNSVNITFPNPNQYKVEFNTPDDQITT
PYIVVVNGHIDPNSKGDLALRSTLYGYNSNIIWRSMSWDNEVAFNNGSG
SGDGIDKPVVPEQPDEPGEIEPIPExDKDHSAPNSRPIDFEMKKKDGTQ
QFYHYASSVKPARVIFTDSKPEIELGLQSGQFWRKFEVYEGDKKLPIKL
VSYDTVKDYAYIRFSVSNGTKAVKIVSSTHFNNKEEKYDYTLMEFAQPI
YNSADKFKT
SEQ ID NO: 66
SLAVAEPVVNAADAKGTNVNDKVTASNFKLEKTTFDPNQSGNTFMAANF
TVTDKVKSGDYFTAKLPDSLTGNGDVDYSNSNNTMPIADIKSTNGDVVA
KATYDILTKTYTFVFTDYVNNKENINGQFSLPLFTDRAKAPKSGTYDAN
INIADEMFNNKITYNYSSPIAGIDKPNGANISSQIIGVDTASGQNTYKQ
TVFVNPKQRVLGNTWVYIKGYQDKIEESSGKVSATDTKLRIFEVNDTSK
LSDSYYADPNDSNLKEVTDQFKNRIYYEHPNVASIKFGDITKTYVVLVE
GHYDNTGKNLKTQVIQENVDPVTNRDYSIFGWNNENVVRYGGGSADGDS
AVNxDKDHSAPNSRPIDFEMKKKDGTQQFYHYASSVKPARVIFTDSKPE
IELGLQSGQFWRKFEVYEGDKKLPIKLVSYDTVKDYAYIRFSVSNGTKA
VKIVSSTHFNNKEEKYDYTLMEFAQPIYNSADKFKT
SEQ ID NO: 67
VAAPQQGTNVNDKVHFSNIDIAIDKGHVNQTTGKTEFWATSSDVLKLKA
NYTIDDSVKEGDTFTFKYGQYFRPGSVRLPSQTQNLYNAQGNIIAKGIY
DSTTNTTTYTFTNYVDQYTNVRGSFEQVAFAKRKNATTDKTAYKMEVTL
GNDTYSEEIIVDYGNKKAQPLISSTNYINNEDLSRNMTAYVNQPKNTYT
KQTFVTNLTGYKFNPNAKNFKIYEVTDQNQFVDSFTPDTSKLKDVTDQF
DVIYSNDNxDKDHSAPNSRPIDFEMKKKDGTQQFYHYASSVKPARVIFT
DSKPEIELGLQSGQFWRKFEVYEGDKKLPIKLVSYDTVKDYAYIRFSVS
NGTKAVKIVSSTHFNNKEEKYDYTLMEFAQPIYNSADKFKT
SEQ ID NO: 68
IAAVQPSSTEAKNVNDLITSNTTLTVVDADKNNKIVPAQDYLSLKSQIT
VDDKVKSGDYFTIKYSDTVQVYGLNPEDIKNIGDIKDPNNGETIATAKH
DTANNLITYTFTDYVDRFNSVQMGINYSIYMDADTIPVSKNDVEFNVTI
GNTTTKTTANIQYPDYVVNEKNSIGSAFTETVSHVGNKENPGYYKQTIY
VNPSENSLTNAKLKVQAYHSSYPNNIGQINKDVTDIKIYQVPKGYTLNK
GYDVNTKELTDVTNQYLQKITYGDNNSAVIDFGNADSAYVVMVNTKFQY
TNSESPTLVQMATLSSTGNKSVSTGNALGFTNNQSGGAGQExDKDHSAP
NSRPIDFEMKKKDGTQQFYHYASSVKPARVIFTDSKPEIELGLQSGQFW
RKFEVYEGDKKLPIKLVSYDTVKDYAYIRFSVSNGTKAVKIVSSTHFNN
KEEKYDYTLMEFAQPIYNSADKFKT
SEQ ID NO: 69
FAVAQPAAVASNNVNDLITVTKQTIKVGDGKDNVAAAHDGKDIEYDTEF
TIDNKVKKGDTMTINYDKNVIPSDLTDKNDPIDITDPSGEVIAKGTFDK
ATKQITYTFTDYVDKYEDIKARLTLYSYIDKQAVPNETSLNLTFATAGK
ETSQNVSVDYQDPMVHGDSNIQSIFTKLDENKQTIEQQIYVNPLKKTAT
NTKVDIAGSQVDDYGNIKLGNGSTIIDQNTEIKVYKVNPNQQLPQSNRI
YDFSQYEDVTSQFDNKKSFSNNVATLDFGDINSAYIIKVVSKYTPTSDG
ELDIAQGTSMRTTDKYGYYNYAGYSNFIVTSNDTGGGDGTVxDKDHSAP
NSRPIDFEMKKKDGTQQFYHYASSVKPARVIFTDSKPEIELGLQSGQFW
RKFEVYEGDKKLPIKLVSYDTVKDYAYIRFSVSNGTKAVKIVSSTHFNN
KEEKYDYTLMEFAQPIYNSADKFKT
SEQ ID NO: 70
VTVNQLAAEQGSNVNHLIKVTDQSITEGYDDSEGVIKAHDAENLIYDVT
FEVDDKVKSGDTMTVDIDKNTVPSDLTDSFTIPKIKDNSGEIIATGTYD
NKNKQITYTFTDYVDKYENIKAHLKLTSYIDKSKVPNNNTKLDVEYKTA
LSSVNKTITVEYQRPNENRTANLQSMFTNIDTKNHTVEQTIYINPLRYS
AKETNVNISGNGDEGSTIIDDSTIIKVYKVGDNQNLPDSNRIYDYSEYE
DVTNDDYAQLGNNNxDKDHSAPNSRPIDFEMKKKDGTQQFYHYASSVKP
ARVIFTDSKPEIELGLQSGQFWRKFEVYEGDKKLPIKLVSYDTVKDYAY
IRFSVSNGTKAVKIVSSTHFNNKEEKYDYTLMEFAQPIYNSADKFKT
SEQ ID NO: 71
PTNEKMTDLQDTKYVVYESVENNESMMDTFVKHPIKTGMLNGKKYMVME
TTNDDYWKDFMVEGQRVRTISKDAKNNTRTIIFPYVEGKTLYDAIVKVH
VKTIDYDGQYHVRIVDKEAFTKANTDKSxSLAAVAADAPVAGTDITNQL
TNVTVGIDSGTTVYPHQAGYVKLNYGFSVPNSAVKGDTFKITVPKELNL
NGVTSTAKVPPIMAGDQVLANGVIDSDGNVIYTFTDYVNTKDDVKATLT
MPAYIDPENVKKTGNVTLATGIGSTTANKTVLVDYEKYGKFYNLSIKGT
IDQIDKTNNTYRQTIYVNPSGDNVIAPVLTGNLKPNTDSNALIDQQNTS
IKVYKVDNAADLSESYFVNPENFEDVTNSVNITFPNPNQYKVEFNTPDD
QITTPYIVVVNGHIDPNSKGDLALRSTLYGYNSNIIWRSMSWDNEVAFN
NGSGSGDGIDKPVVPEQPDEPGEIEPIPE
SEQ ID NO: 72
PTNEKMTDLQDTKYVVYESVENNESMMDTFVKHPIKTGMLNGKKYMVME
TTNDDYWKDFMVEGQRVRTISKDAKNNTRTIIFPYVEGKTLYDAIVKVH
VKTIDYDGQYHVRIVDKEAFTKANTDKSxSLAVAEPVVNAADAKGTNVN
DKVTASNFKLEKTTFDPNQSGNTFMAANFTVTDKVKSGDYFTAKLPDSL
TGNGDVDYSNSNNTMPIADIKSTNGDVVAKATYDILTKTYTFVFTDYVN
NKENINGQFSLPLFTDRAKAPKSGTYDANINIADEMFNNKITYNYSSPI
AGIDKPNGANISSQIIGVDTASGQNTYKQTVFVNPKQRVLGNTWVYIKG
YQDKIEESSGKVSATDTKLRIFEVNDTSKLSDSYYADPNDSNLKEVTDQ
FKNRIYYEHPNVASIKFGDITKTYVVLVEGHYDNTGKNLKTQVIQENVD
PVTNRDYSIFGWNNENVVRYGGGSADGDSAVN
SEQ ID NO: 73
PTNEKMTDLQDTKYVVYESVENNESMMDTFVKHPIKTGMLNGKKYMVME
TTNDDYWKDFMVEGQRVRTISKDAKNNTRTIIFPYVEGKTLYDAIVKVH
VKTIDYDGQYHVRIVDKEAFTKANTDKSxVAAPQQGTNVNDKVHFSNID
IAIDKGHVNQTTGKTEFWATSSDVLKLKANYTIDDSVKEGDTFTFKYGQ
YFRPGSVRLPSQTQNLYNAQGNIIAKGIYDSTTNTTTYTFTNYVDQYTN
VRGSFEQVAFAKRKNATTDKTAYKMEVTLGNDTYSEEIIVDYGNKKAQP
LISSTNYINNEDLSRNMTAYVNQPKNTYTKQTFVTNLTGYKFNPNAKNF
KIYEVTDQNQFVDSFTPDTSKLKDVTDQFDVIYSNDN
SEQ ID NO: 74
PTNEKMTDLQDTKYVVYESVENNESMMDTFVKHPIKTGMLNGKKYMVME
TTNDDYWKDFMVEGQRVRTISKDAKNNTRTIIFPYVEGKTLYDAIVKVH
VKTIDYDGQYHVRIVDKEAFTKANTDKSxIAAVQPSSTEAKNVNDLITS
NTTLTVVDADKNNKIVPAQDYLSLKSQITVDDKVKSGDYFTIKYSDTVQ
VYGLNPEDIKNIGDIKDPNNGETIATAKHDTANNLITYTFTDYVDRFNS
VQMGINYSIYMDADTIPVSKNDVEFNVTIGNTTTKTTANIQYPDYVVNE
KNSIGSAFTETVSHVGNKENPGYYKQTIYVNPSENSLTNAKLKVQAYHS
SYPNNIGQINKDVTDIKIYQVPKGYTLNKGYDVNTKELTDVTNQYLQKI
TYGDNNSAVIDFGNADSAYVVMVNTKFQYTNSESPTLVQMATLSSTGNK
SVSTGNALGFTNNQSGGAGQE
SEQ ID NO: 75
PTNEKMTDLQDTKYVVYESVENNESMMDTFVKHPIKTGMLNGKKYMVME
TTNDDYWKDFMVEGQRVRTISKDAKNNTRTIIFPYVEGKTLYDAIVKVH
VKTIDYDGQYHVRIVDKEAFTKANTDKSxFAVAQPAAVASNNVNDLITV
TKQTIKVGDGKDNVAAAHDGKDIEYDTEFTIDNKVKKGDTMTINYDKNV
IPSDLTDKNDPIDITDPSGEVIAKGTFDKATKQITYTFTDYVDKYEDIK
ARLTLYSYIDKQAVPNETSLNLTFATAGKETSQNVSVDYQDPMVHGDSN
IQSIFTKLDENKQTIEQQIYVNPLKKTATNTKVDIAGSQVDDYGNIKLG
NGSTIIDQNTEIKVYKVNPNQQLPQSNRIYDFSQYEDVTSQFDNKKSFS
NNVATLDFGDINSAYIIKVVSKYTPTSDGELDIAQGTSMRTTDKYGYYN
YAGYSNFIVTSNDTGGGDGTV
SEQ ID NO: 76
PTNEKMTDLQDTKYVVYESVENNESMMDTFVKHPIKTGMLNGKKYMVME
TTNDDYWKDFMVEGQRVRTISKDAKNNTRTIIFPYVEGKTLYDAIVKVH
VKTIDYDGQYHVRIVDKEAFTKANTDKSxVTVNQLAAEQGSNVNHLIKV
TDQSITEGYDDSEGVIKAHDAENLIYDVTFEVDDKVKSGDTMTVDIDKN
TVPSDLTDSFTIPKIKDNSGEIIATGTYDNKNKQITYTFTDYVDKYENI
KAHLKLTSYIDKSKVPNNNTKLDVEYKTALSSVNKTITVEYQRPNENRT
ANLQSMFTNIDTKNHTVEQTTYINPLRYSAKETNVNISGNGDEGSTIID
DSTIIKVYKVGDNQNLPDSNRIYDYSEYEDVTNDDYAQLGNNN
SEQ ID NO: 77
SLAAVAADAPVAGTDITNQLTNVTVGIDSGTTVYPHQAGYVKLNYGFSV
PNSAVKGDTFKITVPKELNLNGVTSTAKVPPIMAGDQVLANGVIDSDGN
VIYTFTDYVNTKDDVKATLTMPAYIDPENVKKTGNVTLATGIGSTTANK
TVLVDYEKYGKFYNLSIKGTIDQIDKTNNTYRQTIYVNPSGDNVIAPVL
TGNLKPNTDSNALIDQQNTSIKVYKVDNAADLSESYFVNPENFEDVTNS
VNITFPNPNQYKVEFNTPDDQITTPYIVVVNGHIDPNSKGDLALRSTLY
GYNSNIIWRSMSWDNEVAFNNGSGSGDGIDKPVVPEQPDEPGEIEPIPE
xPTNEKMTDLQDTKYVVYESVENNESMMDTFVKHPIKTGMLNGKKYMVM
ETTNDDYWKDFMVEGQRVRTISKDAKNNTRTIIFPYVEGKTLYDAIVKV
HVKTIDYDGQYHVRIVDKEAFTKANTDKS
SEQ ID NO: 78
SLAVAEPVVNAADAKGTNVNDKVTASNFKLEKTTFDPNQSGNTFMAANF
TVTDKVKSGDYFTAKLPDSLTGNGDVDYSNSNNTMPIADIKSTNGDVVA
KATYDILTKTYTFVFTDYVNNKENINGQFSLPLFTDRAKAPKSGTYDAN
INIADEMFNNKITYNYSSPIAGIDKPNGANISSQIIGVDTASGQNTYKQ
TVFVNPKQRVLGNTWVYIKGYQDKIEESSGKVSATDTKLRIFEVNDTSK
LSDSYYADPNDSNLKEVTDQFKNRIYYEHPNVASIKFGDITKTYVVLVE
GHYDNTGKNLKTQVIQENVDPVTNRDYSIFGWNNENVVRYGGGSADGDS
AVNxPTNEKMTDLQDTKYVVYESVENNESMMDTFVKHPIKTGMLNGKKY
MVMETTNDDYWKDFMVEGQRVRTISKDAKNNTRTIIFPYVEGKTLYDAI
VKVHVKTIDYDGQYHVRIVDKEAFTKANTDKS
SEQ ID NO: 79
VAAPQQGTNVNDKVHFSNIDIAIDKGHVNQTTGKTEFWATSSDVLKLKA
NYTIDDSVKEGDTFTFKYGQYFRPGSVRLPSQTQNLYNAQGNIIAKGIY
DSTTNTTTYTFTNYVDQYTNVRGSFEQVAFAKRKNATTDKTAYKMEVTL
GNDTYSEEIIVDYGNKKAQPLISSTNYINNEDLSRNMTAYVNQPKNTYT
KQTFVTNLTGYKFNPNAKNFKIYEVTDQNQFVDSFTPDTSKLKDVTDQF
DVIYSNDNxPTNEKMTDLQDTKYVVYESVENNESMMDTFVKHPIKTGML
NGKKYMVMETTNDDYWKDFMVEGQRVRTISKDAKNNTRTIIFPYVEGKT
LYDAIVKVHVKTIDYDGQYHVRIVDKEAFTKANTDKS
SEQ ID NO: 80
IAAVQPSSTEAKNVNDLITSNTTLTVVDADKNNKIVPAQDYLSLKSQIT
VDDKVKSGDYFTIKYSDTVQVYGLNPEDIKNIGDIKDPNNGETIATAKH
DTANNLITYTFTDYVDRFNSVQMGINYSIYMDADTIPVSKNDVEFNVTI
GNTTTKTTANIQYPDYVVNEKNSIGSAFTETVSHVGNKENPGYYKQTIY
VNPSENSLTNAKLKVQAYHSSYPNNIGQINKDVTDIKIYQVPKGYTLNK
GYDVNTKELTDVTNQYLQKITYGDNNSAVIDFGNADSAYVVMVNTKFQY
TNSESPTLVQMATLSSTGNKSVSTGNALGFTNNQSGGAGQExPTNEKMT
DLQDTKYVVYESVENNESMMDTFVKHPIKTGMLNGKKYMVMETTNDDYW
KDFMVEGQRVRTISKDAKNNTRTIIFPYVEGKTLYDAIVKVHVKTIDYD
GQYHVRIVDKEAFTKANTDKS
SEQ ID NO: 81
FAVAQPAAVASNNVNDLITVTKQTIKVGDGKDNVAAAHDGKDIEYDTEF
TIDNKVKKGDTMTINYDKNVIPSDLTDKNDPIDITDPSGEVIAKGTFDK
ATKQITYTFTDYVDKYEDIKARLTLYSYIDKQAVPNETSLNLTFATAGK
ETSQNVSVDYQDPMVHGDSNIQSIFTKLDENKQTIEQQIYVNPLKKTAT
NTKVDIAGSQVDDYGNIKLGNGSTIIDQNTEIKVYKVNPNQQLPQSNRI
YDFSQYEDVTSQFDNKKSFSNNVATLDFGDINSAYIIKVVSKYTPTSDG
ELDIAQGTSMRTTDKYGYYNYAGYSNFIVTSNDTGGGDGTVxPTNEKMT
DLQDTKYVVYESVENNESMMDTFVKHPIKTGMLNGKKYMVMETTNDDYW
KDFMVEGQRVRTISKDAKNNTRTIIFPYVEGKTLYDAIVKVHVKTIDYD
GQYHVRIVDKEAFTKANTDKS
SEQ ID NO: 82
VTVNQLAAEQGSNVNHLIKVTDQSITEGYDDSEGVIKAHDAENLIYDVT
FEVDDKVKSGDTMTVDIDKNTVPSDLTDSFTIPKIKDNSGEIIATGTYD
NKNKQITYTFTDYVDKYENIKAHLKLTSYIDKSKVPNNNTKLDVEYKTA
LSSVNKTITVEYQRPNENRTANLQSMFTNIDTKNHTVEQTIYINPLRYS
AKETNVNISGNGDEGSTIIDDSTIIKVYKVGDNQNLPDSNRIYDYSEYE
DVTNDDYAQLGNNNxPTNEKMTDLQDTKYVVYESVENNESMMDTFVKHP
IKTGMLNGKKYMVMETTNDDYWKDFMVEGQRVRTISKDAKNNTRTIIFP
YVEGKTLYDAIVKVHVKTIDYDGQYHVRIVDKEAFTKANTDKS
SEQ ID NO: 83
STQVSQATSQPINFQVQKDGSSEKSHMDDYMQHPGKVIKQNNKYYFQTV
LNNASFWKEYKFYNANNQELATTVVNDNKKADTRTINVAVEPGYKSLTT
KVHIVVPQINYNHRYTTHLEFEKAIPTLADAAKxDKDHSAPNSRPIDFE
MKKKDGTQQFYHYASSVKPARVIFTDSKPEIELGLQSGQFWRKFEVYEG
DKKLPIKLVSYDTVKDYAYIRFSVSNGTKAVKIVSSTHFNNKEEKYDYT
LMEFAQPIYNSADKFKTEEDYKAEKLLAPYKKAKTLERQVYELNKIQDK
LPEKLKAEYKKKLEDTKKALDEQVKSAITEFQNVQPTNEKMTDLQDTKY
VVYESVENNESMMDTFVKHPIKTGMLNGKKYMVMETTNDDYWKDFMVEG
QRVRTISKDAKNNTRTIIFPYVEGKTLYDAIVKVHVKTIDYDGQYHVRI
VDKEAFTKANTDKS
SEQ ID NO: 84
DKDHSAPNSRPIDFEMKKKDGTQQFYHYASSVKPARVIFTDSKPEIELG
LQSGQFWRKFEVYEGDKKLPIKLVSYDTVKDYAYIRFSVSNGTKAVKIV
SSTHFNNKEEKYDYTLMEFAQPIYNSADKFKTEEDYKAEKLLAPYKKAK
TLERQVYELNKIQDKLPEKLKAEYKKKLEDTKKALDEQVKSAITEFQNV
QPTNEKMTDLQDTKYVVYESVENNESMMDTFVKHPIKTGMLNGKKYMVM
ETTNDDYWKDFMVEGQRVRTISKDAKNNTRTIIFPYVEGKTLYDAIVKV
HVKTIDYDGQYHVRIVDKEAFTKANTDKSxSTQVSQATSQPINFQVQKD
GSSEKSHMDDYMQHPGKVIKQNNKYYFQTVLNNASFWKEYKFYNANNQE
LATTVVNDNKKADTRTINVAVEPGYKSLTTKVHIVVPQINYNHRYTTHL
EFEKAIPTLADAAK
SEQ ID NO: 85
DQTKTQTAHTVKTAQTAQEQNKVQTPVKDVATAKSESNNQAVSDNKSQQ
TNKVTKHNETPKQASKAKELPKTGLTSVDNFISTVAFATLALLGSLSLL
LFKRKESK
SEQ ID NO: 86
MKKKDGTQQFYHYASSVKPARVIFTDSKPEIELGLQSGQFWRKFEVYEG
DKKLPIKLVSYDTVKDYAYIRFSVSNGTKAVKIVSSTHFNNKEEKYDYT
LMEFAQPIYNSADKFKTEEDYKAEKLLAPYKKAKTLERQVYELNKIQDK
LPEKLKAEYKKKLEDTKKALDEQVKSAITEFQNVQPTNEKMTDLQDTKY
VVYESVENNESMMDTFVKHPIKTGMLNGKKYMVMETTNDDYWKDFMVEG
QRVRTISKDAKNNTRTIIFPYVEGKTLYDAIVKVHVKTIDYDGQYHVRI
VDKEAFTKANTD
SEQ ID NO: 87
DQTKTQTAHTVKTAQTAQEQNKVQTPVKDVATAKSESNNQAVSDNKSQQ
TNKVTKHNETPKQASKAKELPKTGLTSVDNFISTVAFATLALLGSLSLL
LFKRKESKxMKKKDGTQQFYHYASSVKPARVIFTDSKPEIELGLQSGQF
WRKFEVYEGDKKLPIKLVSYDTVKDYAYIRFSVSNGTKAVKIVSSTHFN
NKEEKYDYTLMEFAQPIYNSADKFKTEEDYKAEKLLAPYKKAKTLERQV
YELNKIQDKLPEKLKAEYKKKLEDTKKALDEQVKSAITEFQNVQPTNEK
MTDLQDTKYVVYESVENNESMMDTFVKHPIKTGMLNGKKYMVMETTNDD
YWKDFMVEGQRVRTISKDAKNNTRTIIFPYVEGKTLYDAIVKVHVKTID
YDGQYHVRIVDKEAFTKAHTD
SEQ ID NO: 88
MKKKDGTQQFYHYASSVKPARVIFTDSKPEIELGLQSGQFWRKFEVYEG
DKKLPIKLVSYDTVKDYAYIRFSVSNGTKAVKIVSSTHFNNKEEKYDYT
LMEFAQPIYNSADKFKTEEDYKAEKLLAPYKKAKTLERQVYELNKIQDK
LPEKLKAEYKKKLEDTKKALDEQVKSAITEFQNVQPTNEKMTDLQDTKY
VVYESVENNESMMDTFVKHPIKTGMLNGKKYMVMETTNDDYWKDFMVEG
QRVRTISKDAKNNTRTIIFPYVEGKTLYDAIVKVHVKTIDYDGQYHVRI
VDKEAFTKAHTDxDQTKTQTAHTVKTAQTAQEQNKVQTPVKDVATAKSE
SNNQAVSDNKSQQTNKVTKHNETPKQASKAKELPKTGLTSVDNFISTVA
FATLALLGSLSLLLFKRKESK
SEQ ID NO: 89
DQTKTQTAHTVKTAQTAQEQNKVQTPVKDVATAKSESNNQAVSDNKSQQ
TNKVTKHNETPKQASKAKELPKTGLTSVDNFISTVAFATLALLGSLSLL
LFKRKESKxMKKKDGTQQFYHYASSVKPARVIFTDSKPEIELGLQSGQF
WRKFEVYEGDKKLPIKLVSYDTVKDYAYIRFSVSNGTKAVKIVSSTHFN
NKEEKYDYTLMEFAQPIYNSADKFKTEEDYKAEKLLAPYKKAKTLERQV
YELNKIQDKLPEKLKAEYKKKLEDTKKALDEQVKSAITEFQNVQPTNEK
MTDLQDTKYVVYESVENNESMMDTFVKHPIKTGMLNGKKYMVMETTNDD
YWKDFMVEGQRVRTISKDAKNNTRTIIFPYVEGKTLYDAIVKVHVKTID
YDGQYHVRIVDKEAFTKANTD
SEQ ID NO: 90
MKKKDGTQQFYHYASSVKPARVIFTDSKPEIELGLQSGQFWRKFEVYEG
DKKLPIKLVSYDTVKDYAYIRFSVSNGTKAVKIVSSTHFNNKEEKYDYT
LMEFAQPIYNSADKFKTEEDYKAEKLLAPYKKAKTLERQVYELNKIQDK
LPEKLKAEYKKKLEDTKKALDEQVKSAITEFQNVQPTNEKMTDLQDTKY
VVYESVENNESMMDTFVKHPIKTGMLNGKKYMVMETTNDDYWKDFMVEG
QRVRTISKDAKNNTRTIIFPYVEGKTLYDAIVKVHVKTIDYDGQYHVRI
VDKEAFTKANTDxDQTKTQTAHTVKTAQTAQEQNKVQTPVKDVATAKSE
SNNQAVSDNKSQQTNKVTKHNETPKQASKAKELPKTGLTSVDNFISTVA
FATLALLGSLSLLLFKRKESK
SEQ ID NO: 91
DQTKTQTAHTVKTAQTAQEQNKVQTPVKDVATAKSESNNQAVSDNKSQQ
TNKVTKHNETPKQASKAKELPKTGLTSVDNFISTVAFATLALLGSLSLL
LFKRKESKxMKKKDGTQQFYHYASSVKPARVIFTDSKPEIELGLQSGQF
WRKFEVYEGDKKLPIKLVSYDTVKDYAYIRFSVSNGTKAVKIVSSTHFN
NKEEKYDYTLMEFAQPIYNSADKFKTxPTNEKMTDLQDTKYVVYESVEN
NESMMDTFVKHPIKTGMLNGKKYMVMETTNDDYWKDFMVEGQRVRTISK
DAKNNTRTIIFPYVEGKTLYDAIVKVHVKTIDYDGQYHVRIVDKEAFTK
AHTD
SEQ ID NO: 92
MKKKDGTQQFYHYASSVKPARVIFTDSKPEIELGLQSGQFWRKFEVYEG
DKKLPIKLVSYDTVKDYAYIRFSVSNGTKAVKIVSSTHFNNKEEKYDYT
LMEFAQPIYNSADKFKTxPTNEKMTDLQDTKYVVYESVENNESMMDTFV
KHPIKTGMLNGKKYMVMETTNDDYWKDFMVEGQRVRTISKDAKNNTRTI
IFPYVEGKTLYDAIVKVHVKTIDYDGQYHVRIVDKEAFTKAHTDxDQTK
TQTAHTVKTAQTAQEQNKVQTPVKDVATAKSESNNQAVSDNKSQQTNKV
TKHNETPKQASKAKELPKTGLTSVDNFISTVAFATLALLGSLSLLLFKR
KESK
SEQ ID NO: 93
DQTKTQTAHTVKTAQTAQEQNKVQTPVKDVATAKSESNNQAVSDNKSQQ
TNKVTKHNETPKQASKAKELPKTGLTSVDNFISTVAFATLALLGSLSLL
LFKRKESKxMKKKDGTQQFYHYASSVKPARVIFTDSKPEIELGLQSGQF
WRKFEVYEGDKKLPIKLVSYDTVKDYAYIRFSVSNGTKAVKIVSSTHFN
NKEEKYDYTLMEFAQPIYNSADKFKTxPTNEKMTDLQDTKYVVYESVEN
NESMMDTFVKHPIKTGMLNGKKYMVMETTNDDYWKDFMVEGQRVRTISK
DAKNNTRTIIFPYVEGKTLYDAIVKVHVKTIDYDGQYHVRIVDKEAFTK
ANTD
SEQ ID NO: 94
MKKKDGTQQFYHYASSVKPARVIFTDSKPEIELGLQSGQFWRKFEVYEG
DKKLPIKLVSYDTVKDYAYIRFSVSNGTKAVKIVSSTHFNNKEEKYDYT
LMEFAQPIYNSADKFKTxPTNEKMTDLQDTKYVVYESVENNESMMDTFV
KHPIKTGMLNGKKYMVMETTNDDYWKDFMVEGQRVRTISKDAKNNTRTI
IFPYVEGKTLYDAIVKVHVKTIDYDGQYHVRIVDKEAFTKANTDxDQTK
TQTAHTVKTAQTAQEQNKVQTPVKDVATAKSESNNQAVSDNKSQQTNKV
TKHNETPKQASKAKELPKTGLTSVDNFISTVAFATLALLGSLSLLLFKR
KESK
SEQ ID NO: 95
DSQQVNAATEATNATNNQSTQVSQATSQPINFQVQKDGSSEKSHMDDYM
QHPGKVIKQNNKYYFQTVLNNASFWKEYKFYNANNQELATTVVNDNKKA
DTRTINVAVEPGYKSLTTKVHIVVPQINYNHRYTTHLEFEKAIPTLADA
AKGGSDKDHSAPNSRPIDFEMKKKDGTQQFYHYASSVKPARVIFTDSKP
EIELGLQSGQFWRKFEVYEGDKKLPIKLVSYDTVKDYAYIRFSVSNGTK
AVKIVSSTHFNNKEEKYDYTLMEFAQPIYNSADKFKTDTNDAVVTNDQS
SSVASNQTNTNTSNQNISTINNANNQPQATTNMSQPAQPKSSTNADQAS
SQPAHETNSNGNTNDKTNESSNQSDVNQQYPPADESLQDAIKNPAIIPT
NEKMTDLQDTKYVVYESVENNESMMDTFVKHPIKTGMLNGKKYMVMETT
NDDYWKDFMVEGQRVRTISKDAKNNTRTIIFPYVEGKTLYDAIVKVHVK
TIDYDGQYHVRIVDKEAFTKANTDKS
SEQ ID NO: 96
DSQQVNAATEATNATNNQSTQVSQATSQPINFQVQKDGSSEKSHMDDYM
QHPGKVIKQNNKYYFQTVLNNASFWKEYKFYNANNQELATTVVNDNKKA
DTRTINVAVEPGYKSLTTKVHIVVPQINYNHRYTTHLEFEKAIPTLADA
AKDTNDAVVTNDQSSSVASNQTNTNTSNQNISTINNANNQPQATTNMSQ
PAQPKSSTNADQASSQPAHETNSNGNTNDKTNESSNQSDVNQQYPPADE
SLQDAIKNPAIIDKDHSAPNSRPIDFEMKKKDGTQQFYHYASSVKPARV
IFTDSKPEIELGLQSGQFWRKFEVYEGDKKLPIKLVSYDTVKDYAYIRF
SVSNGTKAVKIVSSTHFNNKEEKYDYTLMEFAQPIYNSADKFKTGGSPT
NEKMTDLQDTKYVVYESVENNESMMDTFVKHPIKTGMLNGKKYMVMETT
NDDYWKDFMVEGQRVRTISKDAKNNTRTIIFPYVEGKTLYDAIVKVHVK
TIDYDGQYHVRIVDKEAFTKANTDKS
SEQ ID NO: 97
DSQQVNAATEATNATNNQSTQVSQATSQPINFQVQKDGSSEKSHMDDYM
QHPGKVIKQNNKYYFQTVLNNASFWKEYKFYNANNQELATTVVNDNKKA
DTRTINVAVEPGYKSLTTKVHIVVPQINYNHRYTTHLEFEKAIPTLADA
AKGSGGSDKDHSAPNSRPIDFEMKKKDGTQQFYHYASSVKPARVIFTDS
KPEIELGLQSGQFWRKFEVYEGDKKLPIKLVSYDTVKDYAYIRFSVSNG
TKAVKIVSSTHFNNKEEKYDYTLMEFAQPIYNSADKFKTKLGGSPTNEK
MTDLQDTKYVVYESVENNESMMDTFVKHPIKTGMLNGKKYMVMETTNDD
YWKDFMVEGQRVRTISKDAKNNTRTIIFPYVEGKTLYDAIVKVHVKTID
YDGQYHVRIVDKEAFTKANTDKS
SEQ ID NO: 98
DTNDAVVTNDQSSSVASNQTNTNTSNQNISTINNANNQPQATTNMSQPA
QPKSSTNADQASSQPAHETNSNGNTNDKTNESSNQSDVNQQYPPADESL
QDAIKNPAII

1. An immunogenic composition comprising a fragment of a staphylococcal Isd protein which comprises a NEAT domain.

2. The immunogenic composition of claim 1 wherein the staphylococcal Isd protein is from Staphylococcus aureus.

3. The immunogenic composition of claim 1 or 2 wherein the staphylococcal Isd protein is selected from the group consisting of IsdA, IsdB, IsdH and IsdC.

4. The immunogenic composition of claim 3 wherein the fragment of a staphylococcal Isd protein is amino acids 58-188 of IsdA.

5. The immunogenic composition of claim 3 wherein the fragment of a staphylococcal Isd protein is amino acids 140-269 of IsdB.

6. The immunogenic composition of claim 3 wherein the fragment of a staphylococcal Isd protein is amino acids 337-462 of IsdB.

7. The immunogenic composition of claim 3 wherein the fragment of a staphylococcal Isd protein is amino acids 140-462 of IsdB, optionally having amino acids 269-337 deleted or replaced.

8. The immunogenic composition of claim 6 or 7 wherein at least part, optionally all, of the N-terminal NEAT domain of IsdB (amino acids 140-269) is not present.

9. The immunogenic composition of claim 3 wherein the fragment of a staphylococcal Isd protein is amino acids 23-154 or 28-154 of IsdC.

10. The immunogenic composition of claim 3 wherein the fragment of a staphylococcal Isd protein is amino acids 101-232 of IsdH.

11. The immunogenic composition of claim 3 wherein the fragment of a staphylococcal Isd protein is amino acids 341-471 of IsdH.

12. The immunogenic composition of claim 3 wherein the fragment of a staphylococcal Isd protein is amino acids 539-664 of IsdH.

13. The immunogenic composition of any one of claims 1-12 wherein the fragment has an amino acid sequence having at least 85% identity to an amino acid sequence selected from the group consisting of SEQ ID NO: 2, 3, 4, 5, 7, 8, 9, 10, 11, 12, 13, 15, 17, 18 and 19, or an immunogenic fragment thereof.

14. The immunogenic composition of any one of claims 1-13, comprising a staphylococcal extracellular component binding protein or fragment thereof selected from the group consisting of laminin receptor, SitC/MntC/saliva binding protein, EbhA, EbhB, Elastin binding protein (EbpS), EFB (FIB), SBI, autolysin, ClfA, SdrC, SdrD, SdrE, SdrG, SdrH, Lipase GehD, SasA, FnbA, FnbB, Cna, ClfB, FbpA, Npase, IsaA/PisA, SsaA, EPB, SSP-1, SSP-2, Vitronectin binding protein, fibrinogen binding protein, coagulase, Fig and MAP.

15. The immunogenic composition of claim 14 wherein the staphylococcal extracellular component binding protein is selected from the group consisting of ClfA, ClfB, SdrD, SdrE, SdrG or SdrC or immunogenic fragment thereof.

16. The immunogenic composition of claim 14 wherein the staphylococcal extracellular binding component is a fragment comprising a ligand binding region.

17. The immunogenic composition of any one of claims 1-16 comprising the N23 domain of ClfA.

18. The immunogenic composition of any one of claims 1-17 comprising the N23 domain of ClfB.

19. The immunogenic composition of any one of claims 1-18 comprising the N23 domain of SdrC.

20. The immunogenic composition of any one of claims 1-19 comprising the N23 domain of SdrD.

21. The immunogenic composition of any one of claims 1-20 comprising the N23 domain of SdrE.

22. The immunogenic composition of any one of claims 1-21 comprising the N23 domain of SdrG.

23. The immunogenic composition of claim 14 wherein the fragment comprising a ligand binding region has an amino acid sequence having at least 85% identity to an amino acids sequence selected from the group consisting of SEQ ID NO: 21, 22, 23, 24, 26, 27, 28, 30, 31, 33, 34, 36, 37, 39, 40, 42, 43, 45 and 46 or an immunogenic fragment thereof.

24. The immunogenic composition of any one of claims 14-23 wherein the fragment of a staphylococcal Isd protein is covalently linked to a staphylococcal extracellular component binding protein or fragment thereof.

25. An immunogenic composition comprising a fragment of ClfB, SdrC, SdrD, SdrE or SdrG which comprises a N23 domain.

26. The immunogenic composition of any one of claims 1-25 comprising a further staphylococcal antigen.

27. The immunogenic composition of claim 26 wherein the further staphylococcal antigen comprises S. aureus type 5 and/or 8 capsular saccharide, optionally conjugated to a carrier protein.

28. The immunogenic composition of any one of claims 26-27 wherein the further staphylococcal antigen comprises PNAG optionally conjugated to a carrier protein.

29. The immunogenic composition of claim 28 wherein the PNAG is less than 50%, 40%, 30%, 20% or 10% N-acetylated.

30. The immunogenic composition of any one of claims 26-29 wherein the further staphylococcal antigen is an Isd protein or immunogenic fragment thereof or the fragment of any one of claims 1-24.

31. The immunogenic composition of any one of claims 1-30 comprising an EsxA, EsxB or a combination of EsxA and EsxB.

32. The immunogenic composition of claim 31 wherein the EsxA or EsxB is conjugated to a saccharide antigen.

33. The immunogenic composition of any one of claims 1-32 comprising an EsaC or EsaB antigen.

34. A fusion protein comprising a NEAT domain of a staphylococcal Isd protein involved in an iron/heme uptake system and a ligand binding domain of a staphylococcal extracellular component binding protein.

35. The fusion protein of claim 34 wherein the NEAT domain is from a S. aureus Isd protein optionally selected from the group consisting of IsdA, IsdB, IsdC and IsdH.

36. The fusion protein of claim 34 or 35 wherein the ligand binding domain is from a S. aureus protein optionally selected from the group consisting of ClfA, ClfB, SdrC, SdrD and SdrE.

37. The fusion protein of claim 36 wherein the ligand binding domain consists of the N23 domain of ClfA, ClfB, SdrC, SdrD or SdrE.

38. The fusion protein of claim 36 wherein the ligand binding domain consists of the N123 domain of ClfA, ClfB, SdrC, SdrD or SdrE.

39. The fusion protein of claim 34 or 35 wherein the ligand binding domain is from a S. epidermidis protein, optionally from SdrG.

40. The fusion protein of claim 39 wherein the ligand binding domain consists of the N23 domain of SdrG.

41. The fusion protein of claim 39 wherein the ligand binding domain consists of the N123 domain of SdrG.

42. The fusion protein of any one of claims 34-41 having an amino acid sequence having at least 85% identitiy to an amino acid sequence selected from the group consisting of SEQ ID NO: 47-97.

43. A fusion protein comprising a NEAT domain of a first staphylococcal Isd protein and a NEAT domain from a second Isd protein.

44. The fusion protein of claim 43 wherein the first staphylococcal Isd protein is IsdA.

45. The fusion protein of claim 43 wherein the first staphylococcal Isd protein is IsdB.

46. The fusion protein of claim 45 wherein the NEAT domain from IsdB is the C terminal NEAT domain (optionally comprising amino acids 337-462 of IsdB).

47. The fusion protein of claim 46 wherein at least part, optionally all, of the N-terminal NEAT domain of IsdB (amino acids 140-269) is not present in the fusion protein.

48. The fusion protein of claim 43 wherein the first staphylococcal Isd protein is IsdC.

49. The fusion protein of claim 43 wherein the first staphylococcal Isd protein is IsdH.

50. The fusion protein of any one of claim 43 or 45-49 wherein the second staphylococcal Isd protein is IsdA.

51. The fusion protein of any one of claim 43, 44, 48 or 49 wherein the second staphylococcal Isd protein is IsdB.

52. The fusion protein of claim 51 wherein the second Isd protein is the C-terminal NEAT domain of IsdB.

53. The fusion protein of claim 51 or 52 wherein at least part, optionally all, of the N-terminal NEAT domain of IsdB (amino acids 140-269) is not present in the fusion protein.

54. The fusion protein of any one of claim 43-47 or 49 wherein the second staphylococcal Isd protein is IsdC.

55. The fusion protein of any one of claims 43-48 wherein the second staphylococcal Isd protein is IsdH.

56. The fusion protein of any one of claims 43-55 wherein the first staphylococcal Isd protein is N-terminal to the second staphylococcal Isd protein.

57. The fusion protein of any one of claims 43-55 wherein the first staphylococcal Isd protein is C-terminal to the second staphylococcal Isd protein.

58. The fusion protein of any one of claims 43-56 wherein having an amino acid sequence having at least 85% identitiy to an amino acid sequence selected from the group consisting of SEQ ID NO: 47-97.

59. A fusion protein comprising a ligand binding domain of a first staphylococcal extracellular component binding protein and a ligand binding domain of a second staphylococcal extracellular component binding protein.

60. The fusion protein of claim 58 wherein the first staphylococcal extracellular component binding protein is selected from the group consisting of SdrC, SdrD, SdrE, SdrG, ClfA and ClfB.

61. The fusion protein of claim 58 or 59 wherein the second staphylococcal extracellular component binding protein is selected from the group consisting of SdrC, SdrD, SdrE, SdrG, ClfA and ClfB.

62. The fusion protein of any one of claims 58-60 wherein the ligand binding domain of the first staphylococcal extracellular component binding protein consists of a N2N3 domain.

63. The fusion protein of any one of claims 58-61 wherein the ligand binding domain of the second staphylococcal extracellular component binding protein consists of a N2N3 domain.

64. A polynucleotide comprising a polynucleotide sequence encoding a NEAT domain of a staphylococcal Isd protein and a polynucleotide sequence encoding a ligand binding domain of a staphylococcal extracellular component binding protein.

65. The polynucleotide of claim 63 encoding a sequence having at least 85% identity to a polypeptide sequence selected from the group consisting of SEQ ID NO:47-82.

66. A vaccine comprising the immunogenic composition of any one of claims 1-33, the fusion protein of any one of claims 34-62 or the polynucleotide of any one of claims 63-64 and a pharmaceutically acceptable excipient.

67. A process for making the vaccine of claim 65 comprising the step of adding a pharmaceutically acceptable excipient to the immunogenic composition of any one of claims 1-33, the fusion protein of any one of claims 34-62 or the polynucleotide of any one of claims 63-64.

68. An immunogenic composition according to any one of claims 1-33 for use in the treatment or prevention of staphylococcal infection or disease.

69. A use of the immunogenic composition of any one of claims 1-33 or the fusion protein or any one of claims 34-62 or the polynucleotide of any one of claims 63-64 in the preparation of a medicament for the treatment or prevention of staphylococcal disease.

70. A method of treating or preventing staphylococcal disease comprising administering the immunogenic composition of any one of claims 1-33, the fusion protein of any one of claims 34-62 or the polynucleotide of any one of claims 63-64 to a patient in need thereof.


SEQUENCE LISTING (98 sequences)

SEQ ID NO: 1 - protein, 350 aa, Staphylococcal aureus
MTKHYLNSKYQSEQRSSAMKKITMGTASIILGSLVYIGADSQQVNAATEATNATNNQSTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAKPNNVKPVQPKPAQPKTPTEQTKPVQPKVEKVKPTVTTTSKVEDNHSTKVVSTDTTKDQTKTQTAHTVKTAQTAQEQNKVQTPVKDVATAKSESNNQAVSDNKSQQTNKVTKHNETPKQASKAKELPKTGLTSVDNFISTVAFATLALLGSLSLLLFKRKESK

SEQ ID NO: 2 - protein, 131 aa, Staphylococcal aureus
STQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAK

SEQ ID NO: 3 - protein, 125 aa, Staphylococcal aureus
SQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADA

SEQ ID NO: 4 - protein, 149 aa, Staphylococcal aureus
DSQQVNAATEATNATNNQSTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAK

SEQ ID NO: 5 - protein, 123 aa, Staphylococcal aureus
SQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLA

SEQ ID NO: 6 - protein, 645 aa, Staphylococcal aureus
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 7 - protein, 130 aa, Staphylococcal aureus
DKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKT

SEQ ID NO: 8 - protein, 121 aa, Staphylococcal aureus
SAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSA

SEQ ID NO: 9 - protein, 126 aa, Staphylococcal aureus
PTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKS

SEQ ID NO: 10 - protein, 119 aa, Staphylococcal aureus
KMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANT

SEQ ID NO: 11 - protein, 323 aa, Staphylococcal aureus
DKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKS

SEQ ID NO: 12 - protein, 316 aa, Staphylococcal aureus
SAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANT

SEQ ID NO: 13 - protein, 261 aa, Staphylococcal aureus
DKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTKLGGSPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKS

SEQ ID NO: 14 - protein, 227 aa, Staphylococcal aureus
MKNILKVFNTTILALIIIIATFSNSANAADSGTLNYEVYKYNTNDTSIANDYFNKPAKYIKKNGKLYVQITVNHSHWITGMSIEGHKENIISKNTAKDERTSEFEVSKLNGKIDGKIDVYIDEKVNGKPFKYDHHYNITYKFNGPTDVAGANAPGKDDKNSASGSDKGSDGTTTGQSESNSSNKDKVENPQTNAGTPAYIYAIPVASLALLIAITLFVRKKSKGNVE

SEQ ID NO: 15 - protein, 131 aa, Staphylococcal aureus
NSANAADSGTLNYEVYKYNTNDTSIANDYFNKPAKYIKKNGKLYVQITVNHSHWITGMSIEGHKENIISKNTAKDERTSEFEVSKLNGKIDGKIDVYIDEKVNGKPFKYDHHYNITYKFNGPTDVAGANAP

SEQ ID NO: 16 - protein, 895 aa, Staphylococcal aureus
MNKHHPKLRSFYSIRKSTLGVASVIVSTLFLITSQHQAQAAENTNTSDKISENQNNNATTTQPPKDTNQTQPATQPANTAKNYPAADESLKDAIKDPALENKEHDIGPREQVNFQLLDKNNETQYYHFFSIKDPADVYYTKKKAEVELDINTASTWKKFEVYENNQKLPVRLVSYSPVPEDHAYIRFPVSDGTQELKIVSSTQIDDGEETNYDYTKLVFAKPIYNDPSLVKSDTNDAVVTNDQSSSVASNQTNTNTSNQNISTINNANNQPQATTNMSQPAQPKSSTNADQASSQPAHETNSNGNTNDKTNESSNQSDVNQQYPPADESLQDAIKNPAIIDKEHTADNWRPIDFQMKNDKGERQFYHYASTVEPATVIFTKTGPIIELGLKTASTWKKFEVYEGDKKLPVELVSYDSDKDYAYIRFPVSNGTREVKIVSSIEYGENIHEDYDYTLMVFAQPITNNPDDYVDEETYNLQKLLAPYHKAKTLERQVYELEKLQEKLPEKYKAEYKKKLDQTRVELADQVKSAVTEFENVTPTNDQLTDLQEAHFVVFESEENSESVMDGFVEHPFYTATLNGQKYVVMKTKDDSYWKDLIVEGKRVTTVSKDPKNNSRTLIFPYIPDKAVYNAIVKVVVANIGYEGQYHVRIINQDINTKDDDTSQNNTSEPLNVQTGQEGKVADTDVAENSSTATNPKDASDKADVIEPESDVVKDADNNIDKDVQHDVDHLSDMSDNNHFDKYDLKEMDTQIAKDTDRNVDKDADNSVGMSSNVDTDKDSNKNKDKVIQLNHIADKNNHTGKAAKLDVVKQNYNNTDKVTDKKTTEHLPSDIHKTVDKTVKTKEKAGTPSKENKLSQSKMLPKTGETTSSQSWWGLYALLGMLALFIPKFRKESK

SEQ ID NO: 17 - protein, 132 aa, Staphylococcal aureus
NKEHDIGPREQVNFQLLDKNNETQYYHFFSIKDPADVYYTKKKAEVELDINTASTWKKFEVYENNQKLPVRLVSYSPVPEDHAYIRFPVSDGTQELKIVSSTQIDDGEETNYDYTKLVFAKPIYNDPSLVKS

SEQ ID NO: 18 - protein, 130 aa, Staphylococcal aureus
KEHTADNWRPIDFQMKNDKGERQFYHYASTVEPATVIFTKTGPIIELGLKTASTWKKFEVYEGDKKLPVELVSYDSDKDYAYIRFPVSNGTREVKIVSSIEYGENIHEDYDYTLMVFAQPITNNPDDYVD

SEQ ID NO: 19 - protein, 125 aa, Staphylococcal aureus
TNDQLTDLQEAHFVVFESEENSESVMDGFVEHPFYTATLNGQKYVVMKTKDDSYWKDLIVEGKRVTTVSKDPKNNSRTLIFPYIPDKAVYNAIVKVVVANIGYEGQYHVRIINQDINTKDDDTSQ

SEQ ID NO: 20 - protein, 927 aa, Staphylococcal aureus
MNMKKKEKHAIRKKSIGVASVLVGTLIGFGLLSSKEADASENSVTQSDSASNESKSNDSSSVSAAPKTDDTNVSDTKTSSNTNNGETSVAQNPAQQETTQSSSTNATTEETPVTGEATTTTTNQANTPATTQSSNTNAEELVNQTSNETTSNDTNTVSSVNSPQNSTNAENVSTTQDTSTEATPSNNESAPQSTDASNKDVVNQAVNTSAPRMRAFSLAAVAADAPVAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPEDSDSDPGSDSGSDSNSDSGSDSGSDSTSDSGSDSASDSDSASDSDSASDSDSASDSDSASDSDSDNDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSASDSDSDSDSDSDSDSDSDSDSDSDSDSDSDSDSDSDSDSESDSDSDSDSDSDSDSDSDSDSDSASDSDSGSDSDSSSDSDSESDSNSDSESVSNNNVVPPNSPKNGTNASNKNEAKDSKEPLPDTGSEDEANTSLIWGLLASIGSLLLFRRKKENKDKK

SEQ ID NO: 21 - protein, 559 aa, Staphylococcal aureus
MNMKKKEKHAIRKKSIGVASVLVGTLIGFGLLSSKEADASENSVTQSDSASNESKSNDSSSVSAAPKTDDTNVSDTKTSSNTNNGETSVAQNPAQQETTQSSSTNATTEETPVTGEATTTTTNQANTPATTQSSNTNAEELVNQTSNETTSNDTNTVSSVNSPQNSTNAENVSTTQDTSTEATPSNNESAPQSTDASNKDVVNQAVNTSAPRMRAFSLAAVAADAPVAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPE

SEQ ID NO: 22 - protein, 343 aa, Staphylococcal aureus
SLAAVAADAPVAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPE

SEQ ID NO: 23 - protein, 331 aa, Staphylococcal aureus
GTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPE

SEQ ID NO: 24 - protein, 333 aa, Staphylococcal aureus
MAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMSAAIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPE

SEQ ID NO: 25 - protein, 877 aa, Staphylococcal aureus
LKKRIDYLSNKQNKYSIRRFTVGTTSVIVGATILFGIGNHQAQASEQSNDTTQSSKNNASADSEKNNMIETPQLNTTANDTSDISANTNSANVDSTTKPMSTQTSNTTTTEPASTNETPQPTAIKNQATAAKMQDQTVPQEANSQVDNKTTNDANSIATNSELKNSQTLDLPQSSPQTISNAQGTSKPSVRTRAVRSLAVAEPVVNAADAKGTNVNDKVTASNFKLEKTTFDPNQSGNTFMAANFTVTDKVKSGDYFTAKLPDSLTGNGDVDYSNSNNTMPIADIKSTNGDVVAKATYDILTKTYTFVFTDYVNNKENINGQFSLPLFTDRAKAPKSGTYDANINIADEMFNNKITYNYSSPIAGIDKPNGANISSQIIGVDTASGQNTYKQTVFVNPKQRVLGNTWVYIKGYQDKIEESSGKVSATDTKLRIFEVNDTSKLSDSYYADPNDSNLKEVTDQFKNRIYYEHPNVASIKFGDITKTYVVLVEGHYDNTGKNLKTQVIQENVDPVTNRDYSIFGWNNENVVRYGGGSADGDSAVNPKDPTPGPPVDPEPSPDPEPEPTPDPEPSPDPEPEPSPDPDPDSDSDSDSGSDSDSGSDSDSESDSDSDSDSDSDSDSDSESDSDSESDSESDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSRVTPPNNEQKAPSNPKGEVNHSNKVSKQHKTDALPETGDKSENTNATLFGAMMALLGSLLLFRKRKQDHKEKA

SEQ ID NO: 26 - protein, 542 aa, Staphylococcal aureus
LKKRIDYLSNKQNKYSIRRFTVGTTSVIVGATILFGIGNHQAQASEQSNDTTQSSKNNASADSEKNNMIETPQLNTTANDTSDISANTNSANVDSTTKPMSTQTSNTTTTEPASTNETPQPTAIKNQATAAKMQDQTVPQEANSQVDNKTTNDANSIATNSELKNSQTLDLPQSSPQTISNAQGTSKPSVRTRAVRSLAVAEPVVNAADAKGTNVNDKVTASNFKLEKTTFDPNQSGNTFMAANFTVTDKVKSGDYFTAKLPDSLTGNGDVDYSNSNNTMPIADIKSTNGDVVAKATYDILTKTYTFVFTDYVNNKENINGQFSLPLFTDRAKAPKSGTYDANINIADEMFNNKITYNYSSPIAGIDKPNGANISSQIIGVDTASGQNTYKQTVFVNPKQRVLGNTWVYIKGYQDKIEESSGKVSATDTKLRIFEVNDTSKLSDSYYADPNDSNLKEVTDQFKNRIYYEHPNVASIKFGDITKTYVVLVEGHYDNTGKNLKTQVIQENVDPVTNRDYSIFGWNNENVVRYGGGSADGDSAVN

SEQ ID NO: 27 - protein, 346 aa, Staphylococcal aureus
SLAVAEPVVNAADAKGTNVNDKVTASNFKLEKTTFDPNQSGNTFMAANFTVTDKVKSGDYFTAKLPDSLTGNGDVDYSNSNNTMPIADIKSTNGDVVAKATYDILTKTYTFVFTDYVNNKENINGQFSLPLFTDRAKAPKSGTYDANINIADEMFNNKITYNYSSPIAGIDKPNGANISSQIIGVDTASGQNTYKQTVFVNPKQRVLGNTWVYIKGYQDKIEESSGKVSATDTKLRIFEVNDTSKLSDSYYADPNDSNLKEVTDQFKNRIYYEHPNVASIKFGDITKTYVVLVEGHYDNTGKNLKTQVIQENVDPVTNRDYSIFGWNNENVVRYGGGSADGDSAVN

SEQ ID NO: 28 - protein, 331 aa, Staphylococcal aureus
GTNVNDKVTASNFKLEKTTFDPNQSGNTFMAANFTVTDKVKSGDYFTAKLPDSLTGNGDVDYSNSNNTMPIADIKSTNGDVVAKATYDILTKTYTFVFTDYVNNKENINGQFSLPLFTDRAKAPKSGTYDANINIADEMFNNKITYNYSSPIAGIDKPNGANISSQIIGVDTASGQNTYKQTVFVNPKQRVLGNTWVYIKGYQDKIEESSGKVSATDTKLRIFEVNDTSKLSDSYYADPNDSNLKEVTDQFKNRIYYEHPNVASIKFGDITKTYVVLVEGHYDNTGKNLKTQVIQENVDPVTNRDYSIFGWNNENVVRYGGGSADGDSAVN

SEQ ID NO: 29 - protein, 953 aa, Staphylococcal aureus
MNNKKTATNRKGMIPNRLNKFSIRKYSVGTASILVGTTLIFGLSGHEAKAAEHTNGELNQSKNETTAPSENKTTEKVDSRQLKDNTQTATADQPKVTMSDSATVKETSSNMQSPQNATASQSTTQTSNVTTNDKSSTTYSNETDKSNLTQAKNVSTTPKTTTIKQRALNRMAVNTVAAPQQGTNVNDKVHFTNIDIAIDKGHVNKTTGNTEFWATSSDVLKLKANYTIDDSVKEGDTFTFKYGQYFRPGSVRLPSQTQNLYNAQGNIIAKGIYDSKTNTTTYTFTNYVDQYTNVSGSFEQVAFAKRENATTDKTAYKMEVTLGNDTYSKDVIVDYGNQKGQQLISSTNYINNEDLSRNMTVYVNQPKKTYTKETFVTNLTGYKFNPDAKNFKIYEVTDQNQFVDSFTPDTSKLKDVTGQFDVIYSNDNKTATVDLLNGQSSSDKQYIIQQVAYPDNSSTDNGKIDYTLETQNGKSSWSNSYSNVNGSSTANGDQKKYNLGDYVWEDTNKDGKQDANEKGIKGVYVILKDSNGKELDRTTTDENGKYQFTGLSNGTYSVEFSTPAGYTPTTANAGTDDAVDSDGLTTTGVIKDADNMTLDSGFYKTPKYSLGDYVWYDSNKDGKQDSTEKGIKGVKVTLQNEKGEVIGTTETDENGKYRFDNLDSGKYKVIFEKPAGLTQTGTNTTEDDKDADGGEVDVTITDHDDFTLDNGYYEEETSDSDSDSDSDSDSDSDSDSDSDSDSDSDSDSDSDSDSDSDSDSDSDSDSDSDSDSESDSDSDSDSDSDSDSDSDSDSDSDSDSDSDSDSDSDSDSDSDSDSDSDSDNDSDSDSDSDSDSDSDSDSDSDSDSDSDSDSDSDSDSDSDSDSDSDSDSDSDSDSDSDSDSDSDSDSDSDAGKHTPTKPMSTVKDQHKTAKALPETGSENNNSNNGTLFGGLFAALGSLLLFGRRKKQNK

SEQ ID NO: 30 - protein, 378 aa, Staphylococcal aureus
AEHTNGELNQSKNETTAPSENKTTEKVDSRQLKDNTQTATADQPKVTMSDSATVKETSSNMQSPQNATASQSTTQTSNVTTNDKSSTTYSNETDKSNLTQAKNVSTTPKTTTIKQRALNRMAVNTVAAPQQGTNVNDKVHFTNIDIAIDKGHVNKTTGNTEFWATSSDVLKLKANYTIDDSVKEGDTFTFKYGQYFRPGSVRLPSQTQNLYNAQGNIIAKGIYDSKTNTTTYTFTNYVDQYTNVSGSFEQVAFAKRENATTDKTAYKMEVTLGNDTYSKDVIVDYGNQKGQQLISSTNYINNEDLSRNMTVYVNQPKKTYTKETFVTNLTGYKFNPDAKNFKIYEVTDQNQFVDSFTPDTSKLKDVTGQFDVIYSNDN

SEQ ID NO: 31 - protein, 253 aa, Staphylococcal aureus
VAAPQQGTNVNDKVHFTNIDIAIDKGHVNKTTGNTEFWATSSDVLKLKANYTIDDSVKEGDTFTFKYGQYFRPGSVRLPSQTQNLYNAQGNIIAKGIYDSKTNTTTYTFTNYVDQYTNVSGSFEQVAFAKRENATTDKTAYKMEVTLGNDTYSKDVIVDYGNQKGQQLISSTNYINNEDLSRNMTVYVNQPKKTYTKETFVTNLTGYKFNPDAKNFKIYEVTDQNQFVDSFTPDTSKLKDVTGQFDVIYSNDN

SEQ ID NO: 32 - protein, 995 aa, Staphylococcal aureus
MNNKKTATNRKGMIPNRLNKFSIRKYSVGTASILVGTTLIFGLSGHEAKAAEHTNGELNQSKNETTAPSENKTTKKVDSRQLKDNTQTATADQPKVTMSDSATVKETSSNMQSPQNATANQSTTKTSNVTTNDKSSTTYSNETDKSNLTQAKDVSTTPKTTTIKPRTLNRMAVNTVAAPQQGTNVNDKVHFSNIDIAIDKGHVNQTTGKTEFWATSSDVLKLKANYTIDDSVKEGDTFTFKYGQYFRPGSVRLPSQTQNLYNAQGNIIAKGIYDSTTNTTTYTFTNYVDQYTNVRGSFEQVAFAKRKNATTDKTAYKMEVTLGNDTYSEEIIVDYGNKKAQPLISSTNYINNEDLSRNMTAYVNQPKNTYTKQTFVTNLTGYKFNPNAKNFKIYEVTDQNQFVDSFTPDTSKLKDVTDQFDVIYSNDNKTATVDLMKGQTSSNKQYIIQQVAYPDNSSTDNGKIDYTLDTDKTKYSWSNSYSNVNGSSTANGDQKKYNLGDYVWEDTNKDGKQDANEKGIKGVYVILKDSNGKELDRTTTDENGKYQFTGLSNGTYSVEFSTPAGYTPTTANVGTDDAVDSDGLTTTGVIKDADNMTLDSGFYKTPKYSLGDYVWYDSNKDGKQDSTEKGIKGVKVTLQNEKGEVIGTTETDENGKYRFDNLDSGKYKVIFEKPAGLTQTGTNTTEDDKDADGGEVDVTITDHDDFTLDNGYYEEETSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDNDSDSDSDSDSDSDSDSDSDSDSDSDSDSDSDSDSDSDSDSDSDSDSDSDSDSDSDSDSDSDSDSDNDSDSDSDSDSDAGKHTPAKPMSTVKDQHKTAKALPETGSENNNSNNGTLFGGLFAALGSLLLFGRRKKQNK

SEQ ID NO: 33 - protein, 378 aa, Staphylococcal aureus
AEHTNGELNQSKNETTAPSENKTTKKVDSRQLKDNTQTATADQPKVTMSDSATVKETSSNMQSPQNATANQSTTKTSNVTTNDKSSTTYSNETDKSNLTQAKDVSTTPKTTTIKPRTLNRMAVNTVAAPQQGTNVNDKVHFSNIDIAIDKGHVNQTTGKTEFWATSSDVLKLKANYTIDDSVKEGDTFTFKYGQYFRPGSVRLPSQTQNLYNAQGNIIAKGIYDSTTNTTTYTFTNYVDQYTNVRGSFEQVAFAKRKNATTDKTAYKMEVTLGNDTYSEEIIVDYGNKKAQPLISSTNYINNEDLSRNMTAYVNQPKNTYTKQTFVTNLTGYKFNPNAKNFKIYEVTDQNQFVDSFTPDTSKLKDVTDQFDVIYSNDN

SEQ ID NO: 34 - protein, 253 aa, Staphylococcal aureus
VAAPQQGTNVNDKVHFSNIDIAIDKGHVNQTTGKTEFWATSSDVLKLKANYTIDDSVKEGDTFTFKYGQYFRPGSVRLPSQTQNLYNAQGNIIAKGIYDSTTNTTTYTFTNYVDQYTNVRGSFEQVAFAKRKNATTDKTAYKMEVTLGNDTYSEEIIVDYGNKKAQPLISSTNYINNEDLSRNMTAYVNQPKNTYTKQTFVTNLTGYKFNPNAKNFKIYEVTDQNQFVDSFTPDTSKLKDVTDQFDVIYSNDN

SEQ ID NO: 35 - protein, 1349 aa, Staphylococcal aureus
MLNRENKTAITRKGMVSNRLNKFSIRKYTVGTASILVGTTLIFGLGNQEAKAAESTNKELNEATTSASDNQSSDKVDMQQLNQEDNTKNDNQKEMVSSQGNETTSNGNKLIEKESVQSTTGNKVEVSTAKSDEQASPKSTNEDLNTKQTISNQEALQPDLQENKSVVNVQPTNEENKKVDAKTESTTLNVKSDAIKSNDETLVDNNSNSNNENNADIILPKSTAPKRLNTRMRIAAVQPSSTEAKNVNDLITSNTTLTVVDADKNNKIVPAQDYLSLKSQITVDDKVKSGDYFTIKYSDTVQVYGLNPEDIKNIGDIKDPNNGETIATAKHDTANNLITYTFTDYVDRFNSVQMGINYSIYMDADTIPVSKNDVEFNVTIGNTTTKTTANIQYPDYVVNEKNSIGSAFTETVSHVGNKENPGYYKQTIYVNPSENSLTNAKLKVQAYHSSYPNNIGQINKDVTDIKIYQVPKGYTLNKGYDVNTKELTDVTNQYLQKITYGDNNSAVIDFGNADSAYVVMVNTKFQYTNSESPTLVQMATLSSTGNKSVSTGNALGFTNNQSGGAGQEVYKIGNYVWEDTNKNGVQELGEKGVGNVTVTVFDNNTNTKVGEAVTKEDGSYLIPNLPNGDYRVEFSNLPKGYEVTPSKQGNNEELDSNGLSSVITVNGKDNLSADLGIYKPKYNLGDYVWEDTNKNGIQDQDEKGISGVTVTLKDENGNVLKTVTTDADGKYKFTDLDNGNYKVEFTTPEGYTPTTVTSGSDIEKDSNGLTTTGVINGADNMTLDSGFYKTPKYNLGNYVWEDTNKDGKQDSTEKGISGVTVTLKNENGEVLQTTKTDKDGKYQFTGLENGTYKVEFETPSGYTPTQVGSGTDEGIDSNGTSTTGVIKDKDNDTIDSGFYKPTYNLGDYVWEDTNKNGVQDKDEKGISGVTVTLKDENDKVLKTVTTDENGKYQFTDLNNGTYKVEFETPSGYTPTSVTSGNDTEKDSNGLTTTGVIKDADNMTLDSGFYKTPKYSLGDYVWYDSNKDGKQDSTEKGIKDVKVTLLNEKGEVIGTTKTDENGKYCFDNLDSGKYKVIFEKPAGLTQTVTNTTEDDKDADGGEVDVTITDHDDFTLDNGYFEEDTSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDAGKHTPVKPMSTTKDHHNKAKALPETGSENNGSNNATLFGGLFAALGSLLLFGRRKKQNK

SEQ ID NO: 36 - protein, 516 aa, Staphylococcal aureus
AESTNKELNEATTSASDNQSSDKVDMQQLNQEDNTKNDNQKEMVSSQGNETTSNGNKLIEKESVQSTTGNKVEVSTAKSDEQASPKSTNEDLNTKQTISNQEALQPDLQENKSVVNVQPTNEENKKVDAKTESTTLNVKSDAIKSNDETLVDNNSNSNNENNADIILPKSTAPKRLNTRMRIAAVQPSSTEAKNVNDLITSNTTLTVVDADKNNKIVPAQDYLSLKSQITVDDKVKSGDYFTIKYSDTVQVYGLNPEDIKNIGDIKDPNNGETIATAKHDTANNLITYTFTDYVDRFNSVQMGINYSIYMDADTIPVSKNDVEFNVTIGNTTTKTTANIQYPDYVVNEKNSIGSAFTETVSHVGNKENPGYYKQTIYVNPSENSLTNAKLKVQAYHSSYPNNIGQINKDVTDIKIYQVPKGYTLNKGYDVNTKELTDVTNQYLQKITYGDNNSAVIDFGNADSAYVVMVNTKFQYTNSESPTLVQMATLSSTGNKSVSTGNALGFTNNQSGGAGQE

SEQ ID NO: 37 - protein, 335 aa, Staphylococcal aureus
IAAVQPSSTEAKNVNDLITSNTTLTVVDADKNNKIVPAQDYLSLKSQITVDDKVKSGDYFTIKYSDTVQVYGLNPEDIKNIGDIKDPNNGETIATAKHDTANNLITYTFTDYVDRFNSVQMGINYSIYMDADTIPVSKNDVEFNVTIGNTTTKTTANIQYPDYVVNEKNSIGSAFTETVSHVGNKENPGYYKQTIYVNPSENSLTNAKLKVQAYHSSYPNNIGQINKDVTDIKIYQVPKGYTLNKGYDVNTKELTDVTNQYLQKITYGDNNSAVIDFGNADSAYVVMVNTKFQYTNSESPTLVQMATLSSTGNKSVSTGNALGFTNNQSGGAGQE

SEQ ID NO: 38 - protein, 1141 aa, Staphylococcal aureus
MINRDNKKAITKKGMISNRLNKFSIRKYTVGTASILVGTTLIFGLGNQEAKAAENTSTENAKQDDATTSDNKEVVSETENNSTTENDSTNPIKKETNTDSQPEAKEESTTSSTQQQQNNVTATTETKPQNIEKENVKPSTDKTATEDTSVILEEKKAPNYTNNDVTTKPSTSEIQTKPTTPQESTNIENSQPQPTPSKVDNQVTDATNPKEPVNVSKEELKNNPEKLKELVRNDNNTDRSTKPVATAPTSVAPKRLNAKMRFAVAQPAAVASNNVNDLITVTKQTIKVGDGKDNVAAAHDGKDIEYDTEFTIDNKVKKGDTMTINYDKNVIPSDLTDKNDPIDITDPSGEVIAKGTFDKATKQITYTFTDYVDKYEDIKARLTLYSYIDKQAVPNETSLNLTFATAGKETSQNVSVDYQDPMVHGDSNIQSIFTKLDENKQTIEQQIYVNPLKKTATNTKVDIAGSQVDDYGNIKLGNGSTIIDQNTEIKVYKVNPNQQLPQSNRIYDFSQYEDVTSQFDNKKSFSNNVATLDFGDINSAYIIKVVSKYTPTSDGELDIAQGTSMRTTDKYGYYNYAGYSNFIVTSNDTGGGDGTVKPEEKLYKIGDYVWEDVDKDGVQGTDSKEKPMANVLVTLTYPDGTTKSVRTDANGHYEFGGLKDGETYTVKFETPAGYLPTKVNGTTDGEKDSNGSSITVKINGKDDMSLDTGFYKEPKYNLGDYVWEDTNKDGIQDANEPGIKDVKVTLKDSTGKVIGTTTTDASGKYKFTDLDNGNYTVEFETPAGYTPTVKNTTAEDKDSNGLTTTGVIKDADNMTLDSGFYKTPKYSLGDYVWYDSNKDGKQDSTEKGIKDVKVTLLNEKGEVIGTTKTDENGKYRFDNLDSGKYKVIFEKPAGLTQTVTNTTEDDKDADGGEVDVTITDHDDFILDNGYFEEDTSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDAGKHTPVKPMSTTKDHHNKAKALPETGSENNGSNNATLFGGLFAALGSLLLFGRRKKQNK

SEQ ID NO: 39 - protein, 544 aa, Staphylococcal aureus
AENTSTENAKQDDATTSDNKEVVSETENNSTTENDSTNPIKKETNTDSQPEAKEESTTSSTQQQQNNVTATTETKPQNIEKENVKPSTDKTATEDTSVILEEKKAPNYTNNDVTTKPSTSEIQTKPTTPQESTNIENSQPQPTPSKVDNQVTDATNPKEPVNVSKEELKNNPEKLKELVRNDNNTDRSTKPVATAPTSVAPKRLNAKMRFAVAQPAAVASNNVNDLITVTKQTIKVGDGKDNVAAAHDGKDIEYDTEFTIDNKVKKGDTMTINYDKNVIPSDLTDKNDPIDITDPSGEVIAKGTFDKATKQITYTFTDYVDKYEDIKARLTLYSYIDKQAVPNETSLNLTFATAGKETSQNVSVDYQDPMVHGDSNIQSIFTKLDENKQTIEQQIYVNPLKKTATNTKVDIAGSQVDDYGNIKLGNGSTIIDQNTEIKVYKVNPNQQLPQSNRIYDFSQYEDVTSQFDNKKSFSNNVATLDFGDINSAYIIKVVSKYTPTSDGELDIAQGTSMRTTDKYGYYNYAGYSNFIVTSNDTGGGDGTV

SEQ ID NO: 40 - protein, 335 aa, Staphylococcal aureus
FAVAQPAAVASNNVNDLITVTKQTIKVGDGKDNVAAAHDGKDIEYDTEFTIDNKVKKGDTMTINYDKNVIPSDLTDKNDPIDITDPSGEVIAKGTFDKATKQITYTFTDYVDKYEDIKARLTLYSYIDKQAVPNETSLNLTFATAGKETSQNVSVDYQDPMVHGDSNIQSIFTKLDENKQTIEQQIYVNPLKKTATNTKVDIAGSQVDDYGNIKLGNGSTIIDQNTEIKVYKVNPNQQLPQSNRIYDFSQYEDVTSQFDNKKSFSNNVATLDFGDINSAYIIKVVSKYTPTSDGELDIAQGTSMRTTDKYGYYNYAGYSNFIVTSNDTGGGDGTV

SEQ ID NO: 41 - protein, 1141 aa, Staphylococcal aureus; identical to SEQ ID NO: 38

SEQ ID NO: 42 - protein, 476 aa, Staphylococcal aureus
AENTSTENAKQDDATTSDNKEVVSETENNSTTENDSTNPIKKETNTDSQPEAKEESTTSSTQQQQNNVTATTETKPQNIEKENVKPSTDKTATEDTSVILEEKKAPNYTNNDVTTKPSTSEIQTKPTTPQESTNIENSQPQPTPSKVDNQVTDATNPKEPVNVSKEELKNNPEKLKELVRNDNNTDRSTKPVATAPTSVAPKRLNAKMRFAVAQPAAVASNNVNDLITVTKQTIKVGDGKDNVAAAHDGKDIEYDTEFTIDNKVKKGDTMTINYDKNVIPSDLTDKNDPIDITDPSGEVIAKGTFDKATKQITYTFTDYVDKYEDIKARLTLYSYIDKQAVPNETSLNLTFATAGKETSQNVSVDYQDPMVHGDSNIQSIFTKLDENKQTIEQQIYVNPLKKTATNTKVDIAGSQVDDYGNIKLGNGSTIIDQNTEIKVYKVNPNQQLPQSNRIYDFSQYEDVTSQFDNKKSFSNN

SEQ ID NO: 43 - protein, 267 aa, Staphylococcal aureus
FAVAQPAAVASNNVNDLITVTKQTIKVGDGKDNVAAAHDGKDIEYDTEFTIDNKVKKGDTMTINYDKNVIPSDLTDKNDPIDITDPSGEVIAKGTFDKATKQITYTFTDYVDKYEDIKARLTLYSYIDKQAVPNETSLNLTFATAGKETSQNVSVDYQDPMVHGDSNIQSIFTKLDENKQTIEQQIYVNPLKKTATNTKVDIAGSQVDDYGNIKLGNGSTIIDQNTEIKVYKVNPNQQLPQSNRIYDFSQYEDVTSQFDNKKSFSNN

SEQ ID NO: 44 - protein, 1056 aa, Staphylococcal aureus
MINKKNNLLTKKKPIANKSNKYAIRKFTVGTASIVIGATLLFGLGHNEAKAEENSVQDVKDSNTDDELSDSNDQSSDEEKNDVINNNQSINTDDNNQIIKKEETNNYDGIEKRSEDRTESTTNVDENEATFLQKTPQDNTHLTEEEVKESSSVESSNSSIDTAQQPSHTTINREESVQTSDNVEDSHVSDFANSKIKESNTESGKEENTIEQPNKVKEDSTTSQPSGYTNIDEKISNQDELLNLPINEYENKARPLSTTSAQPSIKRVTVNQLAAEQGSNVNHLIKVTDQSITEGYDDSEGVIKAHDAENLIYDVTFEVDDKVKSGDTMTVDIDKNTVPSDLTDSFTIPKIKDNSGEIIATGTYDNKNKQITYTFTDYVDKYENIKAHLKLTSYIDKSKVPNNNTKLDVEYKTALSSVNKTITVEYQRPNENRTANLQSMFTNIDTKNHTVEQTIYINPLRYSAKETNVNISGNGDEGSTIIDDSTIIKVYKVGDNQNLPDSNRIYDYSEYEDVTNDDYAQLGNNNDVNINFGNIDSPYIIKVISKYDPNKDDYTTIQQTVTMQTTINEYTGEFRTASYDNTIAFSTSSGQGQGDLPPEKTYKIGDYVWEDVDKDGIQNTNDNEKPLSNVLVTLTYPDGTSKSVRTDEDGKYQFDGLKNGLTYKITFETPEGYTPTLKHSGTNPALDSEGNSVWVTINGQDDMTIDSGFYQTPKYSLGNYVWYDTNKDGIQGDDEKGISGVKVTLKDENGNIISTTTTDENGKYQFDNLNSGNYIVHFDKPSGMTQTTTDSGDDDEQDADGEEVHVTITDHDDFSIDNGYYDDESDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDNDSDLGNSSDKSTKDKLPDTGANEDYGSKGTLLGTLFAGLGALLLGKRRKNRKNKN

SEQ ID NO: 45 - protein, 475 aa, Staphylococcal aureus
EENSVQDVKDSNTDDELSDSNDQSSDEEKNDVINNNQSINTDDNNQIIKKEETNNYDGIEKRSEDRTESTTNVDENEATFLQKTPQDNTHLTEEEVKESSSVESSNSSIDTAQQPSHTTINREESVQTSDNVEDSHVSDFANSKIKESNTESGKEENTIEQPNKVKEDSTTSQPSGYTNIDEKISNQDELLNLPINEYENKARPLSTTSAQPSIKRVTVNQLAAEQGSNVNHLIKVTDQSITEGYDDSEGVIKAHDAENLIYDVTFEVDDKVKSGDTMTVDIDKNTVPSDLTDSFTIPKIKDNSGEIIATGTYDNKNKQITYTFTDYVDKYENIKAHLKLTSYIDKSKVPNNNTKLDVEYKTALSSVNKTITVEYQRPNENRTANLQSMFTNIDTKNHTVEQTIYINPLRYSAKETNVNISGNGDEGSTIIDDSTIIKVYKVGDNQNLPDSNRIYDYSEYEDVTNDDYAQLGNNN

SEQ ID NO: 46 - protein, 259 aa, Staphylococcal aureus
VTVNQLAAEQGSNVNHLIKVTDQSITEGYDDSEGVIKAHDAENLIYDVTFEVDDKVKSGDTMTVDIDKNTVPSDLTDSFTIPKIKDNSGEIIATGTYDNKNKQITYTFTDYVDKYENIKAHLKLTSYIDKSKVPNNNTKLDVEYKTALSSVNKTITVEYQRPNENRTANLQSMFTNIDTKNHTVEQTIYINPLRYSAKETNVNISGNGDEGSTIIDDSTIIKVYKVGDNQNLPDSNRIYDYSEYEDVTNDDYAQLGNNN

SEQ ID NO: 47 - protein, 474 aa, Staphylococcal aureus
STQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAKSLAAVAADAPVAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPE

SEQ ID NO: 48 - protein, 477 aa, Staphylococcal aureus
STQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAKSLAVAEPVVNAADAKGTNVNDKVTASNFKLEKTTFDPNQSGNTFMAANFTVTDKVKSGDYFTAKLPDSLTGNGDVDYSNSNNTMPIADIKSTNGDVVAKATYDILTKTYTFVFTDYVNNKENINGQFSLPLFTDRAKAPKSGTYDANINIADEMFNNKITYNYSSPIAGIDKPNGANISSQIIGVDTASGQNTYKQTVFVNPKQRVLGNTWVYIKGYQDKIEESSGKVSATDTKLRIFEVNDTSKLSDSYYADPNDSNLKEVTDQFKNRIYYEHPNVASIKFGDITKTYVVLVEGHYDNTGKNLKTQVIQENVDPVTNRDYSIFGWNNENVVRYGGGSADGDSAVN

SEQ ID NO: 49 - protein, 384 aa, Staphylococcal aureus
STQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAKVAAPQQGTNVNDKVHFSNIDIAIDKGHVNQTTGKTEFWATSSDVLKLKANYTIDDSVKEGDTFTFKYGQYFRPGSVRLPSQTQNLYNAQGNIIAKGIYDSTTNTTTYTFTNYVDQYTNVRGSFEQVAFAKRKNATTDKTAYKMEVTLGNDTYSEEIIVDYGNKKAQPLISSTNYINNEDLSRNMTAYVNQPKNTYTKQTFVTNLTGYKFNPNAKNFKIYEVTDQNQFVDSFTPDTSKLKDVTDQFDVIYSNDN

SEQ ID NO: 50 - protein, 466 aa, Staphylococcal aureus
STQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAKIAAVQPSSTEAKNVNDLITSNTTLTVVDADKNNKIVPAQDYLSLKSQITVDDKVKSGDYFTIKYSDTVQVYGLNPEDIKNIGDIKDPNNGETIATAKHDTANNLITYTFTDYVDRFNSVQMGINYSIYMDADTIPVSKNDVEFNVTIGNTTTKTTANIQYPDYVVNEKNSIGSAFTETVSHVGNKENPGYYKQTIYVNPSENSLTNAKLKVQAYHSSYPNNIGQINKDVTDIKIYQVPKGYTLNKGYDVNTKELTDVTNQYLQKITYGDNNSAVIDFGNADSAYVVMVNTKFQYTNSESPTLVQMATLSSTGNKSVSTGNALGFTNNQSGGAGQE

SEQ ID NO: 51 - protein, 466 aa, Staphylococcal aureus
STQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAKFAVAQPAAVASNNVNDLITVTKQTIKVGDGKDNVAAAHDGKDIEYDTEFTIDNKVKKGDTMTINYDKNVIPSDLTDKNDPIDITDPSGEVIAKGTFDKATKQITYTFTDYVDKYEDIKARLTLYSYIDKQAVPNETSLNLTFATAGKETSQNVSVDYQDPMVHGDSNIQSIFTKLDENKQTIEQQIYVNPLKKTATNTKVDIAGSQVDDYGNIKLGNGSTIIDQNTEIKVYKVNPNQQLPQSNRIYDFSQYEDVTSQFDNKKSFSNNVATLDFGDINSAYIIKVVSKYTPTSDGELDIAQGTSMRTTDKYGYYNYAGYSNFIVTSNDTGGGDGTV

SEQ ID NO: 52 - protein, 390 aa, Staphylococcal aureus
STQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAKVTVNQLAAEQGSNVNHLIKVTDQSITEGYDDSEGVIKAHDAENLIYDVTFEVDDKVKSGDTMTVDIDKNTVPSDLTDSFTIPKIKDNSGEIIATGTYDNKNKQITYTFTDYVDKYENIKAHLKLTSYIDKSKVPNNNTKLDVEYKTALSSVNKTITVEYQRPNENRTANLQSMFTNIDTKNHTVEQTIYINPLRYSAKETNVNISGNGDEGSTIIDDSTIIKVYKVGDNQNLPDSNRIYDYSEYEDVTNDDYAQLGNNN

SEQ ID NO: 53 - protein, 474 aa, Staphylococcal aureus
SLAAVAADAPVAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPESTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAK

SEQ ID NO: 54 - protein, 477 aa, Staphylococcal aureus
SLAVAEPVVNAADAKGTNVNDKVTASNFKLEKTTFDPNQSGNTFMAANFTVTDKVKSGDYFTAKLPDSLTGNGDVDYSNSNNTMPIADIKSTNGDVVAKATYDILTKTYTFVFTDYVNNKENINGQFSLPLFTDRAKAPKSGTYDANINIADEMFNNKITYNYSSPIAGIDKPNGANISSQIIGVDTASGQNTYKQTVFVNPKQRVLGNTWVYIKGYQDKIEESSGKVSATDTKLRIFEVNDTSKLSDSYYADPNDSNLKEVTDQFKNRIYYEHPNVASIKFGDITKTYVVLVEGHYDNTGKNLKTQVIQENVDPVTNRDYSIFGWNNENVVRYGGGSADGDSAVNSTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAK

SEQ ID NO: 55 - protein, 384 aa, Staphylococcal aureus
VAAPQQGTNVNDKVHFSNIDIAIDKGHVNQTTGKTEFWATSSDVLKLKANYTIDDSVKEGDTFTFKYGQYFRPGSVRLPSQTQNLYNAQGNIIAKGIYDSTTNTTTYTFTNYVDQYTNVRGSFEQVAFAKRKNATTDKTAYKMEVTLGNDTYSEEIIVDYGNKKAQPLISSTNYINNEDLSRNMTAYVNQPKNTYTKQTFVTNLTGYKFNPNAKNFKIYEVTDQNQFVDSFTPDTSKLKDVTDQFDVIYSNDNSTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAK

SEQ ID NO: 56 - protein, 466 aa, Staphylococcal aureus
IAAVQPSSTEAKNVNDLITSNTTLTVVDADKNNKIVPAQDYLSLKSQITVDDKVKSGDYFTIKYSDTVQVYGLNPEDIKNIGDIKDPNNGETIATAKHDTANNLITYTFTDYVDRFNSVQMGINYSIYMDADTIPVSKNDVEFNVTIGNTTTKTTANIQYPDYVVNEKNSIGSAFTETVSHVGNKENPGYYKQTIYVNPSENSLTNAKLKVQAYHSSYPNNIGQINKDVTDIKIYQVPKGYTLNKGYDVNTKELTDVTNQYLQKITYGDNNSAVIDFGNADSAYVVMVNTKFQYTNSESPTLVQMATLSSTGNKSVSTGNALGFTNNQSGGAGQESTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAK

SEQ ID NO: 57 - protein, 466 aa, Staphylococcal aureus
FAVAQPAAVASNNVNDLITVTKQTIKVGDGKDNVAAAHDGKDIEYDTEFTIDNKVKKGDTMTINYDKNVIPSDLTDKNDPIDITDPSGEVIAKGTFDKATKQITYTFTDYVDKYEDIKARLTLYSYIDKQAVPNETSLNLTFATAGKETSQNVSVDYQDPMVHGDSNIQSIFTKLDENKQTIEQQIYVNPLKKTATNTKVDIAGSQVDDYGNIKLGNGSTIIDQNTEIKVYKVNPNQQLPQSNRIYDFSQYEDVTSQFDNKKSFSNNVATLDFGDINSAYIIKVVSKYTPTSDGELDIAQGTSMRTTDKYGYYNYAGYSNFIVTSNDTGGGDGTVSTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAK

SEQ ID NO: 58 - protein, 390 aa, Staphylococcal aureus
VTVNQLAAEQGSNVNHLIKVTDQSITEGYDDSEGVIKAHDAENLIYDVTFEVDDKVKSGDTMTVDIDKNTVPSDLTDSFTIPKIKDNSGEIIATGTYDNKNKQITYTFTDYVDKYENIKAHLKLTSYIDKSKVPNNNTKLDVEYKTALSSVNKTITVEYQRPNENRTANLQSMFTNIDTKNHTVEQTIYINPLRYSAKETNVNISGNGDEGSTIIDDSTIIKVYKVGDNQNLPDSNRIYDYSEYEDVTNDDYAQLGNNNSTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAK

SEQ ID NO: 59 - protein, 473 aa, Staphylococcal aureus
DKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTSLAAVAADAPVAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPE

SEQ ID NO: 60 - protein, 476 aa, Staphylococcal aureus
DKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTSLAVAEPVVNAADAKGTNVNDKVTASNFKLEKTTFDPNQSGNTFMAANFTVTDKVKSGDYFTAKLPDSLTGNGDVDYSNSNNTMPIADIKSTNGDVVAKATYDILTKTYTFVFTDYVNNKENINGQFSLPLFTDRAKAPKSGTYDANINIADEMFNNKITYNYSSPIAGIDKPNGANISSQIIGVDTASGQNTYKQTVFVNPKQRVLGNTWVYIKGYQDKIEESSGKVSATDTKLRIFEVNDTSKLSDSYYADPNDSNLKEVTDQFKNRIYYEHPNVASIKFGDITKTYVVLVEGHYDNTGKNLKTQVIQENVDPVTNRDYSIFGWNNENVVRYGGGSADGDSAVN

SEQ ID NO: 61 - protein, 383 aa, Staphylococcal aureus
DKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTVAAPQQGTNVNDKVHFSNIDIAIDKGHVNQTTGKTEFWATSSDVLKLKANYTIDDSVKEGDTFTFKYGQYFRPGSVRLPSQTQNLYNAQGNIIAKGIYDSTTNTTTYTFTNYVDQYTNVRGSFEQVAFAKRKNATTDKTAYKMEVTLGNDTYSEEIIVDYGNKKAQPLISSTNYINNEDLSRNMTAYVNQPKNTYTKQTFVTNLTGYKFNPNAKNFKIYEVTDQNQFVDSFTPDTSKLKDVTDQFDVIYSNDN

SEQ ID NO: 62 - protein, 465 aa, Staphylococcal aureus
DKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTIAAVQPSSTEAKNVNDLITSNTTLTVVDADKNNKIVPAQDYLSLKSQITVDDKVKSGDYFTIKYSDTVQVYGLNPEDIKNIGDIKDPNNGETIATAKHDTANNLITYTFTDYVDRFNSVQMGINYSIYMDADTIPVSKNDVEFNVTIGNTTTKTTANIQYPDYVVNEKNSIGSAFTETVSHVGNKENPGYYKQTIYVNPSENSLTNAKLKVQAYHSSYPNNIGQINKDVTDIKIYQVPKGYTLNKGYDVNTKELTDVTNQYLQKITYGDNNSAVIDFGNADSAYVVMVNTKFQYTNSESPTLVQMATLSSTGNKSVSTGNALGFTNNQSGGAGQE

SEQ ID NO: 63 - protein, 465 aa, Staphylococcal aureus
DKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTFAVAQPAAVASNNVNDLITVTKQTIKVGDGKDNVAAAHDGKDIEYDTEFTIDNKVKKGDTMTINYDKNVIPSDLTDKNDPIDITDPSGEVIAKGTFDKATKQITYTFTDYVDKYEDIKARLTLYSYIDKQAVPNETSLNLTFATAGKETSQNVSVDYQDPMVHGDSNIQSIFTKLDENKQTIEQQIYVNPLKKTATNTKVDIAGSQVDDYGNIKLGNGSTIIDQNTEIKVYKVNPNQQLPQSNRIYDFSQYEDVTSQFDNKKSFSNNVATLDFGDINSAYIIKVVSKYTPTSDGELDIAQGTSMRTTDKYGYYNYAGYSNFIVTSNDTGGGDGTV

SEQ ID NO: 64 - protein, 389 aa, Staphylococcal aureus
DKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTVTVNQLAAEQGSNVNHLIKVTDQSITEGYDDSEGVIKAHDAENLIYDVTFEVDDKVKSGDTMTVDIDKNTVPSDLTDSFTIPKIKDNSGEIIATGTYDNKNKQITYTFTDYVDKYENIKAHLKLTSYIDKSKVPNNNTKLDVEYKTALSSVNKTITVEYQRPNENRTANLQSMFTNIDTKNHTVEQTIYINPLRYSAKETNVNISGNGDEGSTIIDDSTIIKVYKVGDNQNLPDSNRIYDYSEYEDVTNDDYAQLGNNN

SEQ ID NO: 65 - protein, 473 aa, Staphylococcal aureus
SLAAVAADAPVAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPEDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKT

SEQ ID NO: 66 - protein, 476 aa, Staphylococcal aureus
SLAVAEPVVNAADAKGTNVNDKVTASNFKLEKTTFDPNQSGNTFMAANFTVTDKVKSGDYFTAKLPDSLTGNGDVDYSNSNNTMPIADIKSTNGDVVAKATYDILTKTYTFVFTDYVNNKENINGQFSLPLFTDRAKAPKSGTYDANINIADEMFNNKITYNYSSPIAGIDKPNGANISSQIIGVDTASGQNTYKQTVFVNPKQRVLGNTWVYIKGYQDKIEESSGKVSATDTKLRIFEVNDTSKLSDSYYADPNDSNLKEVTDQFKNRIYYEHPNVASIKFGDITKTYVVLVEGHYDNTGKNLKTQVIQENVDPVTNRDYSIFGWNNENVVRYGGGSADGDSAVNDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKT

SEQ ID NO: 67 - protein, 383 aa, Staphylococcal aureus
VAAPQQGTNVNDKVHFSNIDIAIDKGHVNQTTGKTEFWATSSDVLKLKANYTIDDSVKEGDTFTFKYGQYFRPGSVRLPSQTQNLYNAQGNIIAKGIYDSTTNTTTYTFTNYVDQYTNVRGSFEQVAFAKRKNATTDKTAYKMEVTLGNDTYSEEIIVDYGNKKAQPLISSTNYINNEDLSRNMTAYVNQPKNTYTKQTFVTNLTGYKFNPNAKNFKIYEVTDQNQFVDSFTPDTSKLKDVTDQFDVIYSNDNDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKT

SEQ ID NO: 68 - protein, 465 aa, Staphylococcal aureus
IAAVQPSSTEAKNVNDLITSNTTLTVVDADKNNKIVPAQDYLSLKSQITVDDKVKSGDYFTIKYSDTVQVYGLNPEDIKNIGDIKDPNNGETIATAKHDTANNLITYTFTDYVDRFNSVQMGINYSIYMDADTIPVSKNDVEFNVTIGNTTTKTTANIQYPDYVVNEKNSIGSAFTETVSHVGNKENPGYYKQTIYVNPSENSLTNAKLKVQAYHSSYPNNIGQINKDVTDIKIYQVPKGYTLNKGYDVNTKELTDVTNQYLQKITYGDNNSAVIDFGNADSAYVVMVNTKFQYTNSESPTLVQMATLSSTGNKSVSTGNALGFTNNQSGGAGQEDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKT

SEQ ID NO: 69 - protein, 465 aa, Staphylococcal aureus
FAVAQPAAVASNNVNDLITVTKQTIKVGDGKDNVAAAHDGKDIEYDTEFTIDNKVKKGDTMTINYDKNVIPSDLTDKNDPIDITDPSGEVIAKGTFDKATKQITYTFTDYVDKYEDIKARLTLYSYIDKQAVPNETSLNLTFATAGKETSQNVSVDYQDPMVHGDSNIQSIFTKLDENKQTIEQQIYVNPLKKTATNTKVDIAGSQVDDYGNIKLGNGSTIIDQNTEIKVYKVNPNQQLPQSNRIYDFSQYEDVTSQFDNKKSFSNNVATLDFGDINSAYIIKVVSKYTPTSDGELDIAQGTSMRTTDKYGYYNYAGYSNFIVTSNDTGGGDGTVDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKT

SEQ ID NO: 70 - protein, 389 aa, Staphylococcal aureus
VTVNQLAAEQGSNVNHLIKVTDQSITEGYDDSEGVIKAHDAENLIYDVTFEVDDKVKSGDTMTVDIDKNTVPSDLTDSFTIPKIKDNSGEIIATGTYDNKNKQITYTFTDYVDKYENIKAHLKLTSYIDKSKVPNNNTKLDVEYKTALSSVNKTITVEYQRPNENRTANLQSMFTNIDTKNHTVEQTIYINPLRYSAKETNVNISGNGDEGSTIIDDSTIIKVYKVGDNQNLPDSNRIYDYSEYEDVTNDDYAQLGNNNDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKT

SEQ ID NO: 71 - protein, 469 aa, Staphylococcal aureus
PTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSSLAAVAADAPVAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPE

SEQ ID NO: 72 - protein, 472 aa, Staphylococcal aureus
PTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSSLAVAEPVVNAADAKGTNVNDKVTASNFKLEKTTFDPNQSGNTFMAANFTVTDKVKSGDYFTAKLPDSLTGNGDVDYSNSNNTMPIADIKSTNGDVVAKATYDILTKTYTFVFTDYVNNKENINGQFSLPLFTDRAKAPKSGTYDANINIADEMFNNKITYNYSSPIAGIDKPNGANISSQIIGVDTASGQNTYKQTVFVNPKQRVLGNTWVYIKGYQDKIEESSGKVSATDTKLRIFEVNDTSKLSDSYYADPNDSNLKEVTDQFKNRIYYEHPNVASIKFGDITKTYVVLVEGHYDNTGKNLKTQVIQENVDPVTNRDYSIFGWNNENVVRYGGGSADGDSAVN

SEQ ID NO: 73 - protein, 379 aa, Staphylococcal aureus
PTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSVAAPQQGTNVNDKVHFSNIDIAIDKGHVNQTTGKTEFWATSSDVLKLKANYTIDDSVKEGDTFTFKYGQYFRPGSVRLPSQTQNLYNAQGNIIAKGIYDSTTNTTTYTFTNYVDQYTNVRGSFEQVAFAKRKNATTDKTAYKMEVTLGNDTYSEEIIVDYGNKKAQPLISSTNYINNEDLSRNMTAYVNQPKNTYTKQTFVTNLTGYKFNPNAKNFKIYEVTDQNQFVDSFTPDTSKLKDVTDQFDVIYSNDN

SEQ ID NO: 74 - protein, 461 aa, Staphylococcal aureus
PTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSIAAVQPSSTEAKNVNDLITSNTTLTVVDADKNNKIVPAQDYLSLKSQITVDDKVKSGDYFTIKYSDTVQVYGLNPEDIKNIGDIKDPNNGETIATAKHDTANNLITYTFTDYVDRFNSVQMGINYSIYMDADTIPVSKNDVEFNVTIGNTTTKTTANIQYPDYVVNEKNSIGSAFTETVSHVGNKENPGYYKQTIYVNPSENSLTNAKLKVQAYHSSYPNNIGQINKDVTDIKIYQVPKGYTLNKGYDVNTKELTDVTNQYLQKITYGDNNSAVIDFGNADSAYVVMVNTKFQYTNSESPTLVQMATLSSTGNKSVSTGNALGFTNNQSGGAGQE

SEQ ID NO: 75 - protein, 461 aa, Staphylococcal aureus
PTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSFAVAQPAAVASNNVNDLITVTKQTIKVGDGKDNVAAAHDGKDIEYDTEFTIDNKVKKGDTMTINYDKNVIPSDLTDKNDPIDITDPSGEVIAKGTFDKATKQITYTFTDYVDKYEDIKARLTLYSYIDKQAVPNETSLNLTFATAGKETSQNVSVDYQDPMVHGDSNIQSIFTKLDENKQTIEQQIYVNPLKKTATNTKVDIAGSQVDDYGNIKLGNGSTIIDQNTEIKVYKVNPNQQLPQSNRIYDFSQYEDVTSQFDNKKSFSNNVATLDFGDINSAYIIKVVSKYTPTSDGELDIAQGTSMRTTDKYGYYNYAGYSNFIVTSNDTGGGDGTV

SEQ ID NO: 76 - protein, 385 aa, Staphylococcal aureus
PTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSVTVNQLAAEQGSNVNHLIKVTDQSITEGYDDSEGVIKAHDAENLIYDVTFEVDDKVKSGDTMTVDIDKNTVPSDLTDSFTIPKIKDNSGEIIATGTYDNKNKQITYTFTDYVDKYENIKAHLKLTSYIDKSKVPNNNTKLDVEYKTALSSVNKTITVEYQRPNENRTANLQSMFTNIDTKNHTVEQTIYINPLRYSAKETNVNISGNGDEGSTIIDDSTIIKVYKVGDNQNLPDSNRIYDYSEYEDVTNDDYAQLGNNN

SEQ ID NO: 77 - protein, 469 aa, Staphylococcal aureus
SLAAVAADAPVAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPEPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKS

SEQ ID NO: 78 - protein, 472 aa, Staphylococcal aureus
SLAVAEPVVNAADAKGTNVNDKVTASNFKLEKTTFDPNQSGNTFMAANFTVTDKVKSGDYFTAKLPDSLTGNGDVDYSNSNNTMPIADIKSTNGDVVAKATYDILTKTYTFVFTDYVNNKENINGQFSLPLFTDRAKAPKSGTYDANINIADEMFNNKITYNYSSPIAGIDKPNGANISSQIIGVDTASGQNTYKQTVFVNPKQRVLGNTWVYIKGYQDKIEESSGKVSATDTKLRIFEVNDTSKLSDSYYADPNDSNLKEVTDQFKNRIYYEHPNVASIKFGDITKTYVVLVEGHYDNTGKNLKTQVIQENVDPVTNRDYSIFGWNNENVVRYGGGSADGDSAVNPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKS

SEQ ID NO: 79 - protein, 379 aa, Staphylococcal aureus
VAAPQQGTNVNDKVHFSNIDIAIDKGHVNQTTGKTEFWATSSDVLKLKANYTIDDSVKEGDTFTFKYGQYFRPGSVRLPSQTQNLYNAQGNIIAKGIYDSTTNTTTYTFTNYVDQYTNVRGSFEQVAFAKRKNATTDKTAYKMEVTLGNDTYSEEIIVDYGNKKAQPLISSTNYINNEDLSRNMTAYVNQPKNTYTKQTFVTNLTGYKFNPNAKNFKIYEVTDQNQFVDSFTPDTSKLKDVTDQFDVIYSNDNPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKS

SEQ ID NO: 80 - protein, 461 aa, Staphylococcal aureus
IAAVQPSSTEAKNVNDLITSNTTLTVVDADKNNKIVPAQDYLSLKSQITVDDKVKSGDYFTIKYSDTVQVYGLNPEDIKNIGDIKDPNNGETIATAKHDTANNLITYTFTDYVDRFNSVQMGINYSIYMDADTIPVSKNDVEFNVTIGNTTTKTTANIQYPDYVVNEKNSIGSAFTETVSHVGNKENPGYYKQTIYVNPSENSLTNAKLKVQAYHSSYPNNIGQINKDVTDIKIYQVPKGYTLNKGYDVNTKELTDVTNQYLQKITYGDNNSAVIDFGNADSAYVVMVNTKFQYTNSESPTLVQMATLSSTGNKSVSTGNALGFTNNQSGGAGQEPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKS

SEQ ID NO: 81 - protein, 461 aa, Staphylococcal aureus
FAVAQPAAVASNNVNDLITVTKQTIKVGDGKDNVAAAHDGKDIEYDTEFTIDNKVKKGDTMTINYDKNVIPSDLTDKNDPIDITDPSGEVIAKGTFDKATKQITYTFTDYVDKYEDIKARLTLYSYIDKQAVPNETSLNLTFATAGKETSQNVSVDYQDPMVHGDSNIQSIFTKLDENKQTIEQQIYVNPLKKTATNTKVDIAGSQVDDYGNIKLGNGSTIIDQNTEIKVYKVNPNQQLPQSNRIYDFSQYEDVTSQFDNKKSFSNNVATLDFGDINSAYIIKVVSKYTPTSDGELDIAQGTSMRTTDKYGYYNYAGYSNFIVTSNDTGGGDGTVPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKS

SEQ ID NO: 82 - protein, 385 aa, Staphylococcal aureus
VTVNQLAAEQGSNVNHLIKVTDQSITEGYDDSEGVIKAHDAENLIYDVTFEVDDKVKSGDTMTVDIDKNTVPSDLTDSFTIPKIKDNSGEIIATGTYDNKNKQITYTFTDYVDKYENIKAHLKLTSYIDKSKVPNNNTKLDVEYKTALSSVNKTITVEYQRPNENRTANLQSMFTNIDTKNHTVEQTIYINPLRYSAKETNVNISGNGDEGSTIIDDSTIIKVYKVGDNQNLPDSNRIYDYSEYEDVTNDDYAQLGNNNPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKS

SEQ ID NO: 83 - protein, 454 aa, Staphylococcal aureus
STQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKS

SEQ ID NO: 84 - protein, 454 aa, Staphylococcal aureus
DKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSSTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAK

SEQ ID NO: 85 - protein, 106 aa, Staphylococcal aureus
DQTKTQTAHTVKTAQTAQEQNKVQTPVKDVATAKSESNNQAVSDNKSQQTNKVTKHNETPKQASKAKELPKTGLTSVDNFISTVAFATLALLGSLSLLLFKRKESK

SEQ ID NO: 86 - protein, 306 aa, Staphylococcal aureus
MKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTD

SEQ ID NO: 87 - protein, 412 aa, Staphylococcal aureus
DQTKTQTAHTVKTAQTAQEQNKVQTPVKDVATAKSESNNQAVSDNKSQQTNKVTKHNETPKQASKAKELPKTGLTSVDNFISTVAFATLALLGSLSLLLFKRKESKMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKAHTD

SEQ ID NO: 88 - protein, 412 aa, Staphylococcal aureus
MKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKAHTDDQTKTQTAHTVKTAQTAQEQNKVQTPVKDVATAKSESNNQAVSDNKSQQTNKVTKHNETPKQASKAKELPKTGLTSVDNFISTVAFATLALLGSLSLLLFKRKESK

SEQ ID NO: 89 - protein, 412 aa, Staphylococcal aureus
DQTKTQTAHTVKTAQTAQEQNKVQTPVKDVATAKSESNNQAVSDNKSQQTNKVTKHNETPKQASKAKELPKTGLTSVDNFISTVAFATLALLGSLSLLLFKRKESKMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTD

SEQ ID NO: 90 - protein, 412 aa, Staphylococcal aureus
MKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDDQTKTQTAHTVKTAQTAQEQNKVQTPVKDVATAKSESNNQAVSDNKSQQTNKVTKHNETPKQASKAKELPKTGLTSVDNFISTVAFATLALLGSLSLLLFKRKESK

SEQ ID NO: 91 - protein, 345 aa, Staphylococcal aureus
DQTKTQTAHTVKTAQTAQEQNKVQTPVKDVATAKSESNNQAVSDNKSQQTNKVTKHNETPKQASKAKELPKTGLTSVDNFISTVAFATLALLGSLSLLLFKRKESKMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKAHTD

SEQ ID NO: 92 - protein, 345 aa, Staphylococcal aureus
MKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKAHTDDQTKTQTAHTVKTAQTAQEQNKVQTPVKDVATAKSESNNQAVSDNKSQQTNKVTKHNETPKQASKAKELPKTGLTSVDNFISTVAFATLALLGSLSLLLFKRKESK

SEQ ID NO: 93 - protein, 345 aa, Staphylococcal aureus
DQTKTQTAHTVKTAQTAQEQNKVQTPVKDVATAKSESNNQAVSDNKSQQTNKVTKHNETPKQASKAKELPKTGLTSVDNFISTVAFATLALLGSLSLLLFKRKESKMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTD

SEQ ID NO: 94 - protein, 345 aa, Staphylococcal aureus
MKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDDQTKTQTAHTVKTAQTAQEQNKVQTPVKDVATAKSESNNQAVSDNKSQQTNKVTKHNETPKQASKAKELPKTGLTSVDNFISTVAFATLALLGSLSLLLFKRKESK

SEQ ID NO: 95 - protein, 516 aa, Staphylococcal aureus
DSQQVNAATEATNATNNQSTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAKGGSDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTDTNDAVVTNDQSSSVASNQTNTNTSNQNISTINNANNQPQATTNMSQPAQPKSSTNADQASSQPAHETNSNGNTNDKTNESSNQSDVNQQYPPADESLQDAIKNPAIIPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKS

SEQ ID NO: 96 - protein, 516 aa, Staphylococcal aureus
DSQQVNAATEATNATNNQSTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAKDTNDAVVTNDQSSSVASNQTNTNTSNQNISTINNANNQPQATTNMSQPAQPKSSTNADQASSQPAHETNSNGNTNDKTNESSNQSDVNQQYPPADESLQDAIKNPAIIDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTGGSPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKS

SEQ ID NO: 97 - protein, 415 aa, Staphylococcal aureus
DSQQVNAATEATNATNNQSTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAKGSGGSDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTKLGGSPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKS

SEQ ID NO: 98 - protein, 108 aa, Staphylococcal aureus
DTNDAVVTNDQSSSVASNQTNTNTSNQNISTINNANNQPQATTNMSQPAQPKSSTNADQASSQPAHETNSNGNTNDKTNESSNQSDVNQQYPPADESLQDAIKNPAII